(19) United States
(12) Patent Application Publication
(10) Pub. No.: US 2024/0226115 A1
(43) Pub. Date: Jul. 11, 2024

(54) AXL INHIBITOR COMPOUNDS

(71) Applicant: ARCUS BIOSCIENCES, INC., Hayward, CA (US)

(72) Inventors: Corinne Nicole FOLEY, San Carlos, CA (US); Manjunath LAMANI, Fremont, CA (US); Manmohan Reddy LELETI, Dublin, CA (US); Dillon Harding MILES, Berkeley, CA (US); Srinivas PALADUGU, Hayward, CA (US); Jay Patrick POWERS, Sisters, OR (US); Shiwei QU, Alameda, CA (US); Ehesan Ul SHARIF, Menlo Park, CA (US); Rebecca Louise GRANGE, Hayward, CA (US); Guiling ZHAO, San Mateo, CA (US)

(21) Appl. No.: 18/555,971

(22) PCT Filed: May 20, 2022
(86) PCT No.: PCT/US2022/030230

§ 371 Date: Oct. 18, 2023

Related U.S. Application Data
(60) Provisional application No. 63/191,636, filed on May 21, 2021.

(51) Int. Cl. A61K 31/553; A61K 31/437; A61K 31/55; A61K 45/06; C07D 471/04

(52) U.S. Cl. CPC A61K 31/553; A61K 31/437; A61K 31/55; A61K 45/06; C07D 471/04

(57) ABSTRACT

Compounds of Formula I that inhibit AXL, and compositions containing the compound(s) and methods for synthesizing the compounds, are described herein. Also described are the use of such compounds and compositions for the treatment of a diverse array of diseases, disorders, and conditions, including cancer- and immune-related disorders that are mediated, at least in part, by AXL.

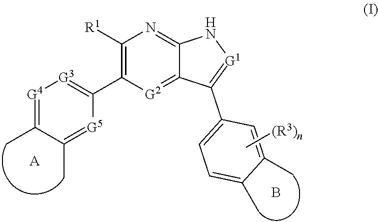

### CROSS-REFERENCES TO RELATED APPLICATIONS

This application claims the benefit of priority under 35 U.S.C. § 119(e) to U.S. Provisional Application Ser. No. 63/191,636 filed May 21, 2021, the disclosure of which is incorporated herein by reference in its entirety for all purposes.

### BACKGROUND OF THE DISCLOSURE

AXL is a receptor tyrosine kinase (RTK) that belongs to the TAM family. AXL regulates important processes such as cell growth, migration, aggregation, and apoptosis. AXL can be activated by a variety of mechanisms including ligand-dependent and ligand-independent mechanisms. Once activated AXL is involved in a variety of signaling pathways including the RAS-RAF-MEK-ERK pathway leading to cancer cell proliferation, and also the PI3K/AKT pathway responsible for several pro-survival proteins.

AXL has been shown to be overexpressed in a variety of malignancies. In cancer settings, AXL overexpression is associated with poor patient survival and resistance mechanisms (both targeted and non-targeted).

In view of the research linking AXL inhibition to diseases such as cancer, there is a need in the art for new AXL inhibitors. The present disclosure addresses this need and provides additional advantages over previous AXL inhibitors.

### BRIEF SUMMARY OF THE DISCLOSURE

The present disclosure relates to compounds that inhibit the activity of AXL. The compounds are represented by Formula (I):

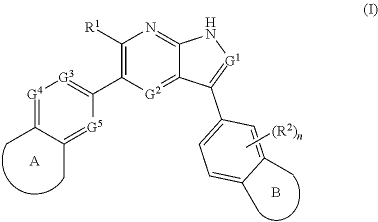

or a pharmaceutically acceptable salt, hydrate, or solvate thereof, wherein R¹, R², the subscript n, fused Rings A and B, and vertices G¹, G², G³, G⁴, and G⁵ have the meanings defined herein below.

In a related aspect, provided herein are methods for treating a disease or disorder mediated by AXL in a subject (e.g., a human) comprising administering to the subject an effective amount of at least one AXL inhibitor described herein. Diseases and disorders mediated by AXL include cancer, inflammation, autoimmune disorders and metabolic disorders, as described hereafter. Other diseases, disorders and conditions that can be treated or prevented, in whole or in part, by modulation of AXL activity are candidate indications for the AXL inhibitor compounds provided herein.

Also provided herein is the use of the described AXL inhibitors in combination with one or more additional agents as hereinafter described.

### DETAILED DESCRIPTION OF THE DISCLOSURE

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. Unless defined otherwise, all technical and scientific terms used herein have the same meaning as commonly understood by one of ordinary skill in the art to which this disclosure belongs.

As used herein, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Definitions

Unless otherwise indicated, the following terms are intended to have the meaning set forth below. Other terms are defined elsewhere throughout the specification.

The term “alkyl”, by itself or as part of another substituent, means, unless otherwise stated, a saturated straight or branched chain hydrocarbon radical, having the number of carbon atoms designated (i.e. C₁₋₈ means one to eight carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

The term “hydroxyalkyl” refers to an alkyl group having the indicated number of carbon atoms (e.g., C₁₋₆ or C₁₋₈) and which is substituted with one or two hydroxy (OH) groups.

The term “halohydroxyalkyl” refers to an alkyl group having the indicated number of carbon atoms (e.g., C₁₋₆ or C₁₋₈) and which is substituted with one or two hydroxy (OH) groups and from one to six halogen atoms (e.g., F, Cl).

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups, in some embodiments, can be substituted or unsubstituted. When a group comprising an alkylene is optionally substituted, it is understood that the optional substitutions may be on the alkylene portion of the moiety.

The term “cycloalkyl” refers to a monocyclic, bicyclic or polycyclic non-aromatic hydrocarbon ring system having the indicated number of ring atoms (e.g., a C₃₋₆ cycloalkyl has from 3 to 6 ring carbon atoms). Cycloalkyl groups can be saturated or partially unsaturated, i.e., cycloalkyl groups can be characterized by one or more points of unsaturation, provided the points of unsaturation do not result in an aromatic system. Examples of monocyclic cycloalkyl groups include, but are not limited to, cyclopropyl, cyclobutyl, cyclopentyl, cyclohexyl, cyclohexenyl, cycloheptyl, cyclooctyl, cyclooctenyl, cyclooctadienyl and the like. “Cycloalkyl” also refers to bicyclic and polycyclic hydrocarbon rings such as, for example, bicyclo[2.2.1]heptane, bicyclo[2.2.2]octane, etc. In some embodiments, the cycloalkyl compounds of the present disclosure are monocyclic C₃₋₆ cycloalkyl moieties.

The term “heterocycloalkyl” refers to a monocyclic, bicyclic or polycyclic cycloalkyl ring having the indicated number of ring vertices (or members) (e.g., 3- to 14-members, or 4- to 10-members, or 4- to 8-members, or 4- to 6-members) and having from one to five heteroatoms selected from N, O, and S in a chemically stable arrangement, which replace one to five of the carbon vertices, and wherein the nitrogen and sulfur atoms are optionally oxidized, and the nitrogen atom(s) are optionally quaternized. Heterocycloalkyl groups can be saturated or partially unsaturated, i.e., heterocycloalkyl groups can be characterized by one or more points of unsaturation, provided the points of unsaturation do not result in an aromatic system. The rings of bicyclic and polycyclic heterocycloalkyl groups can be fused, bridged, or spirocyclic. Non-limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, 3-oxa-6-azabicyclo[3.1.1]heptane, 8-azabicyclo[3.2.1]octane, piperazine, pyran, pyridone, oxetane, 3-pyrroline, thiopyran, pyrone, tetrahydrofuran, tetrahydrothiophene, azetidine, quinuclidine, and the like. The heterocycloalkyl group is attached to the remainder of the molecule through a ring carbon atom. When a heterocycloalkyl is substituted, that substituent is connected to the heterocycloalkyl through a ring carbon atom or a ring heteroatom when chemically permissible.

As used herein, a wavy line, “w”, that intersects a single, double or triple bond in any chemical structure depicted herein, represent the point of attachment of the single, double, or triple bond to the remainder of the molecule. Additionally, a bond extending from a substituent to the center of a ring (e.g., a phenyl ring) is meant to indicate attachment of that substituent to the ring at any of the available ring vertices, i.e., such that the attachment of the substituent to the ring results in a chemically stable arrangement.

As referred to herein, divalent components include either orientation (forward or reverse) of that component. For example, the group “—C(O)NH—” is meant to include a linkage in either orientation: —C(O)NH— or —NHC(O)—, and similarly, “—O—CH₂CH₂—” is meant to include both —O—CH₂CH₂— and —CH₂CH₂—O—.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, and the like.

The term “aryl” means, unless otherwise stated, a monocyclic, bicyclic or tricyclic aromatic hydrocarbon group. Bicyclic and tricyclic ring systems may be fused together or linked covalently. Non-limiting examples of aryl groups include phenyl, naphthyl and biphenyl. The term is also meant to include fused cycloalkylphenyl and heterocycloalkylphenyl ring systems such as, for example, indane, tetrahydronaphthalene, chromane and isochromane rings. As a substituent group, the point of attachment to the remainder of the molecule, for a fused ring system can be through any carbon atom on the aromatic portion, a carbon atom on the cycloalkyl portion, or an atom on the heterocycloalkyl portion.

The term “heteroaryl” refers to monocyclic or fused bicyclic aromatic groups (or rings) that contain from one to five heteroatoms selected from N, O, and S in a chemically stable arrangement, wherein the nitrogen and sulfur atoms are optionally oxidized, and the nitrogen atom(s) are optionally quaternized. A heteroaryl group can be attached to the remainder of the molecule through a heteroatom or a carbon atom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolinyl, isoquinolinyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. When a heteroaryl is substituted, that substituent is connected to the heteroaryl through a ring carbon atom or a ring heteroatom when chemically permissible. Substituents for a heteroaryl ring can be selected from the group of acceptable substituents described below.

As used herein, the term “heteroatom” is meant to include oxygen (O), nitrogen (N), sulfur (S) and silicon (Si). In some embodiments, heteroatom is N, O or S.

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. Certain compounds of the present disclosure may exist in multiple crystalline or amorphous forms.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure. When a stereochemical depiction is shown, it is meant to refer to the compound in which the depicted isomer is present and substantially free of the other isomer(s). “Substantially free of” the other isomer(s) indicates at least an 80/20 ratio of the depicted isomer to the other isomer(s), more preferably 90/10, or 95/5 or more. In some embodiments, one of the isomers will be present in an amount of at least 99%.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics. All isotopic variations of the compounds of the present disclosure, whether radioactive or not, are intended to be encompassed within the scope of the present disclosure.

The terms “patient” or “subject” are used interchangeably to refer to a human or a non-human animal (e.g., a mammal).

The terms “treat”, “treating”, treatment” and the like refer to a course of action (such as administering an inhibitor of AXL or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering an AXL inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent (e.g., a compound according to this disclosure) to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of an AXL inhibitor (or, e.g., a metabolite thereof) at a particular time post-administration may be indicative of whether a therapeutically effective amount has been used. Additionally, a therapeutically effective dose of the AXL inhibitors of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

The terms “inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. An inhibitor may also be defined as a molecule that reduces, blocks, or inactivates a constitutive activity. Activators are molecules that increase, activate, facilitate, enhance activation, sensitize, or up-regulate, e.g., a gene, protein, ligand, receptor, or cell. An “agonist” is a molecule that interacts with a target to cause or promote an increase in the activation of the target. An “antagonist” is a molecule that opposes the action(s) of an agonist.

An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of a particular target, e.g., AXL, either directly or indirectly. A modulator may act alone, or it may use a cofactor, e.g., a protein, metal ion, or small molecule. Examples of modulators include small molecule compounds (e.g., the compounds according to this disclosure) and other bioorganic molecules.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently (though not always) means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect may refer to efficacy, stability, solubility, or immunogenicity.

“Substantially pure” indicates that a component (e.g., a compound according to this disclosure) makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

Compounds that are selective may be particularly useful in the treatment of certain disorders or may offer a reduced likelihood of undesired side effects. In one embodiment, compounds of the present disclosure are selective over other receptor tyrosine kinases (e.g., MER and/or TYRO3). Selectivity may be determined, for example, by comparing the inhibition of a compound as described herein against AXL with the inhibition of the compound against another receptor tyrosine kinase (e.g., MER and/or TYRO3). In one embodiment, the selective inhibition of AXL is at least 1000 times greater, 500 times greater, 100 times greater, 50 times greater, 40 times greater, 30 times greater, or 20 times greater than inhibition of other receptor tyrosine kinases.

### Compounds of the Disclosure

In one particular aspect, provided herein are compounds having Formula (I):

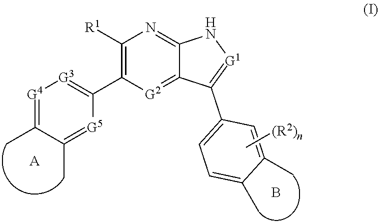

or a pharmaceutically acceptable salt, hydrate, or solvate thereof, wherein:
G¹ is N or CR^{G1};
G² is CR^{G2} or N;
G³ is CR^{G3} or N;
G⁴ is CR^{G4} or N;
G⁵ is CR^{G5} or N;
R^{G1} is selected from the group consisting of H, C₁₋₃ alkyl, halogen, C₁₋₃ haloalkyl and CN;
each R^{G2}, R^{G3}, R^{G4} and R^{G5} is independently selected from the group consisting of H, halo, CN, C₁₋₇ alkyl, C₃₋₇ cycloalkyl, C₁₋₃ haloalkyl, —O—C₁₋₃ alkyl, —O—C₁₋₃ haloalkyl, —NR^{a}R^{b}, and 4- to 8-membered heterocycloalkyl having 1-3 heteroatom ring vertices selected from the group consisting of O, N, and S, and wherein the cycloalkyl and heterocycloalkyl groups are substituted with 0-3 groups independently selected from halo, CN, C₁₋₄ alkyl, C₁₋₄ haloalkyl, C₁₋₄ hydroxyalkyl, —O—C₁₋₄ alkyl, and OH;
R¹ is selected from the group consisting of H, C₁₋₄ alkyl and NH₂;
A is a fused ring selected from the group consisting of azepane, piperidine, cycloheptane, cyclohexane, cyclopentane, 1,4-oxazepane, oxepane, tetrahydropyran, 1,4-diazepane, bicyclo[4.2.1]nonane, bicyclo[4.1.1]octane, spiro[4.6]undecane, 1-azaspiro[4.6]undecane and cyclooctane, each of which is unsubstituted or substituted with from 1 to 4 R², and further substituted with 0 or 1 oxo (═O) which is adjacent to a nitrogen atom;
B is a fused ring selected from the group consisting of 1,4-oxazepane, cycloheptane, tetrahydropyran, isothiazolidine 1,1-dioxide, oxepane, 1,4,5-oxathiazepane 4,4-dioxide, cyclohexane, cyclopentane, azepane, pyrrolidine, piperidine, piperazine, morpholine, diazepane, and 1,3-dioxolane, each of which is unsubstituted or substituted with from 1 to 4 R⁴; and further substituted with 0 or 1 oxo (═O) which is adjacent to a nitrogen atom;
each R² is independently selected from the group consisting of halo, OH, C₁₋₇ alkyl, C₃₋₇ alkenyl, C₃₋₇ alkynyl, C₃₋₇ cycloalkyl, —C(O)—C₁₋₇ alkyl, —C(O)—C₃₋₇ cycloalkyl, —C(O)—C₁₋₇ alkylene-OH, —Y¹—O—C₁₋₇ alkyl, —Y¹—O—C₃₋₇ cycloalkyl, —NR^{a}R^{b}, —S(O)₂—C₁₋₇ alkyl, —S(O)₂—C₃₋₇ cycloalkyl, —C(O)NR^{a}R^{b}, 4- to 8-membered heterocycloalkyl, and —NR^{a}—(4- to 8-membered heterocycloalkyl), wherein the 4- to 8-membered heterocycloalkyl has 1-3 heteroatom ring vertices selected from the group consisting of O, N, and S, and wherein the cycloalkyl and heterocycloalkyl groups are substituted with from 0-3 groups independently selected from halo, CN, C₁₋₄ alkyl, C₁₋₄ haloalkyl, C₁₋₄ hydroxyalkyl, —O—C₁₋₄ alkyl, and OH;
the subscript n is 0, 1, 2 or 3;
each R³ is independently selected from the group consisting of halogen, CN, C₁₋₇ alkyl, C₂₋₇ alkenyl, C₃₋₇ alkynyl, C₃₋₇ cycloalkyl, C₁₋₆ haloalkyl, C₁₋₆ hydroxyalkyl, C₁₋₆ halohydroxyalkyl, —O—C₁₋₇ alkyl, —O—C₃₋₇ cycloalkyl, —O—C₁₋₆ haloalkyl, —X¹—CN, —X¹—O—C₁₋₇ alkyl, —O—Y¹—O—C₁₋₇ alkyl, —NR^{a}R^{b}, —X¹—NR^{a}R^{b}, —O—Y¹—NR^{a}R^{b}, —C(O)—NR^{a}R^{b}, —S(O)₂—NR^{a}R^{b}, —S(O)(NH)—C₁₋₇ alkyl, —S(O)₂—C₁₋₇ alkyl, —S(O)₂—C₁₋₇ haloalkyl, —S(O)₂—C₃₋₇ cycloalkyl, —S(O)₂—Y¹—O—C₁₋₃ alkyl, —S(O)₂-(4- to 8-membered heterocycloalkyl), —C(O)NH-(4- to 8-membered heterocycloalkyl), 4- to 8-membered heterocycloalkyl, and —O—X¹-(4- to 8-membered heterocycloalkyl), wherein the 4- to 8-membered heterocycloalkyl has 1-2 heteroatom ring vertices selected from the group consisting of O, N, and S; and wherein the cycloalkyl and heterocycloalkyl groups are substituted with 0-3 groups independently selected from halo, CN, C₁₋₄ alkyl, C₁₋₄ haloalkyl, C₁₋₄ hydroxyalkyl, —O—C₁₋₄ alkyl, and OH;
each R⁴ is independently selected from the group consisting of H, halogen, hydroxy, CN, C₁₋₇ alkyl, C₂₋₇ alkenyl, C₃₋₇ alkynyl, C₃₋₇ cycloalkyl, C₁₋₆ haloalkyl, C₁₋₆ hydroxyalkyl, C₁₋₆ halohydroxyalkyl, —O—C₁₋₇ alkyl, —O—C₃₋₇ cycloalkyl, —O—C₁₋₆ haloalkyl, —X¹—CN, —X¹—O—C₁₋₇ alkyl, —S(O)₂—C₁₋₄ alkyl, —S(O)₂—C₃₋₇ cycloalkyl, —C(O)NR^{a}R^{b}, —NR^{a}R^{b}, —NR^{a}—C(O)—C₁₋₇ alkyl, —NR^{a}—C(O)—C₃₋₇ cycloalkyl, —NR^{a}—S(O)₂—C₁₋₇ alkyl, and —NR^{a}—S(O)₂—C₃₋₇ cycloalkyl, wherein —NR^{a}R^{b}, —NR^{a}—C(O)—C₁₋₇ alkyl, —NR^{a}—C(O)—C₃₋₇ cycloalkyl, —NR^{a}—S(O)₂—C₁₋₇ alkyl, and —NR^{a}—S(O)₂—C₃₋₇ cycloalkyl groups are not directly attached to a nitrogen ring vertex to form a N—N bond;
or two R⁴ attached to a common carbon are combined to form a C₃₋₆ spirocycloalkyl which is unsubstituted or substituted with 1-3 members independently selected from F, Cl, OH, and CH₃;
each X¹ is C₁₋₇ alkylene or C₃₋₇ cycloalkylene;
each Y¹ is C₂₋₇ alkylene or C₃₋₇ cycloalkylene, wherein two attached heteroatoms are not attached to a common carbon atom;
each R^{a} and R^{b} are independently selected from group consisting of H, C₁₋₇ alkyl, C₁₋₇ haloalkyl, C₁₋₄ alkoxyC₁₋₄alkyl, and C₃₋₇ cycloalkyl; or
R^{a} and R^{b} together with the nitrogen to which they are attached form a 4-8 membered heterocycloalkyl ring having 0-2 additional heteroatom ring vertices selected from the group consisting of O, N, and S, and substituted with 0-3 groups independently selected from halogen, CN, C₁₋₄ alkyl, C₁₋₄ haloalkyl, C₁₋₄ hydroxyalkyl, —O—C₁₋₄ alkyl, oxo and OH.

In some selected embodiments, the compound of Formula (I) or a pharmaceutically acceptable salt, hydrate, or solvate thereof, is a compound wherein G¹ is N. In other selected embodiments, the compound of Formula (I) or a pharmaceutically acceptable salt, hydrate, or solvate thereof, is a compound wherein G¹ is CH.

In some selected embodiments, the compound of Formula (I) or a pharmaceutically acceptable salt, hydrate, or solvate thereof, including any selected embodiments above, is a compound wherein G² is CH or CF.

In some selected embodiments, the compound of Formula (I) or a pharmaceutically acceptable salt, hydrate, or solvate thereof, including selected embodiments above, is a compound wherein G³ is selected from the group consisting of CH, CF, C(CH₃) and N.

In some selected embodiments, the compound of Formula (I) or a pharmaceutically acceptable salt, hydrate, or solvate thereof, including any selected embodiments above, is a compound wherein G⁴ is CH, CCl or N.

In some selected embodiments, the compound of Formula (I) or a pharmaceutically acceptable salt, hydrate, or solvate thereof, including any selected embodiments above, is a compound wherein G⁵ is CH or N.

In some selected embodiments, the compound of Formula (I) or a pharmaceutically acceptable salt, hydrate, or solvate thereof, is a compound wherein G¹ is N and G² is CH. In further selected embodiments, G¹ is N, G² is CH and G³ is CH. In still further selected embodiments, G¹ is N, G² is CH, G³ is CH and G⁴ is CH. In yet further selected embodiments, G¹ is N, G² is CH, G³ is CH, G⁴ is CH and G⁵ is CH.

With reference to ring A, it is understood that ring A is fused to an aromatic ring comprising G³, G⁴ and G⁵, and that the presence of ring A does not disrupt the aromaticity of the aromatic ring. Specifically, the ring vertices that fuse the two rings together are sp² hybridized carbon atoms. Therefore, each of these ring vertices have a p orbital that participates in the conjugated pi system of the the aromatic ring. Accordingly, it is understood that all ring A moieties have a point of unsaturation at the fusion point to the remainder of the molecule. For example, cyclopentane at ring A refers to cyclopentene, where a double bond is between the two carbon atoms that fuse to the remainder of the compound.

Similar to ring A, ring B is fused to an aromatic phenyl ring, and the presence of ring B does not disrupt the aromaticity of the phenyl ring. Accordingly, it is understood that all ring B moieties have a point of unsaturation at the fusion point to the remainder of the molecule. For example, cycloheptane at ring B refers to cycloheptene, where a double bond is between the two carbon atoms that fuse to the remainder of the compound.

In some selected embodiments, the compound of Formula (I) or a pharmaceutically acceptable salt, hydrate, or solvate thereof, including any selected embodiments above, is a compound wherein fused ring A has a formula selected from the group consisting of:

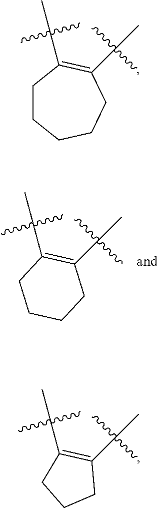

each of which is unsubstituted or substituted with from 1 to 4 R². In still further selected embodiments, fused ring A has the formula:

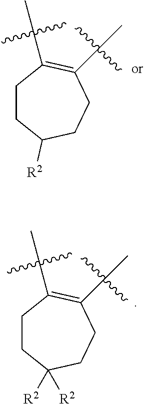

In yet further selected embodiments, the compound of Formula (I) or a pharmaceutically acceptable salt, hydrate, or solvate thereof, including any selected embodiments above, is a compound wherein one R² is —NR^{a}R^{b}. In still further selected embodiments, R^{a} and R^{b} are combined with the nitrogen to which each is attached to form a 4- to 6-membered heterocycloalkyl ring having 0-2 additional heteroatom ring vertices selected from the group consisting of O, N, and S, and substituted with 0-3 groups independently selected from halogen, CN, C₁₋₄ alkyl, C₁₋₄ haloalkyl, C₁₋₄ hydroxyalkyl, —O—C₁₋₄ alkyl, oxo and OH. In further embodiments, R^{a} and R^{b} are combined with the nitrogen to which each is attached to form a pyrrolidinyl ring which is unsubstituted or substituted with 1-3 groups independently selected from halogen, CN, C₁₋₄ alkyl, C₁₋₄ haloalkyl, C₁₋₄ hydroxyalkyl, —O—C₁₋₄ alkyl, oxo and OH.

In some selected embodiments, the compound of Formula (I) or a pharmaceutically acceptable salt, hydrate, or solvate thereof, including any selected embodiments above, is a compound wherein fused ring A has a formula selected from the group consisting of:

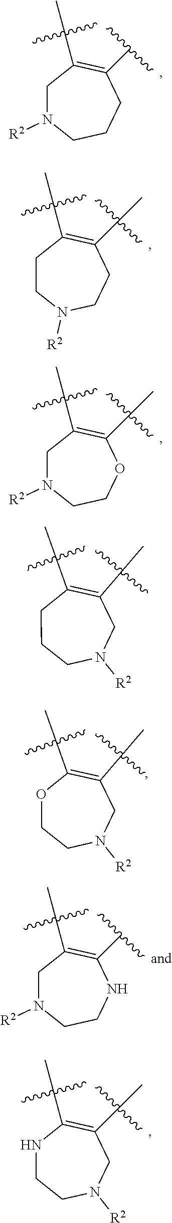

each of which is optionally substituted with an additional 1 to 2 R². In still further selected embodiments, fused ring A has the formula:

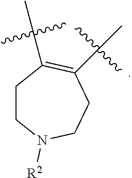

In yet further selected embodiments, the compound of Formula (I) or a pharmaceutically acceptable salt, hydrate, or solvate thereof, including any selected embodiments above, is a compound wherein R² is selected from the group consisting of C₁₋₇ alkyl, C₃₋₇ cycloalkyl, —C(O)—C₁₋₇ alkyl, —C(O)—C₃₋₇ cycloalkyl, —C(O)—C₁₋₇ alkylene-OH, —Y¹—O—C₁₋₇ alkyl, —Y¹—O—C₃₋₇ cycloalkyl, —S(O)₂—C₁₋₇ alkyl, —S(O)₂—C₃₋₇ cycloalkyl, —C(O)NR^{a}R^{b}, and 4- to 8-membered heterocycloalkyl, wherein the 4- to 8-membered heterocycloalkyl has 1-3 heteroatom ring vertices selected from the group consisting of O, N, and S, and wherein the cycloalkyl and heterocycloalkyl groups are substituted with from 0-3 groups independently selected from halo, CN, C₁₋₄ alkyl, C₁₋₄ haloalkyl, C₁₋₄ hydroxyalkyl, —O—C₁₋₄ alkyl, and OH.

In some selected embodiments, the compound of Formula (I) or a pharmaceutically acceptable salt, hydrate, or solvate thereof, including any selected embodiments above, is a compound wherein fused ring B is selected from the group consisting of 1,4-oxazepane, tetrahydropyran, isothiazolidine 1,1-dioxide, 1,4,5-oxathiazepane 4,4-dioxide, azepane, and pyrrolidine, each of which is unsubstituted or substituted with from 1 to 3 R⁴; and further substituted with 0 or 1 oxo (═O) which is adjacent to a nitrogen atom. In further selected embodiments, each R⁴ is independently selected from the group consisting of halogen, C₁₋₄ alkyl, C₁₋₄ haloalkyl and OH, or two R⁴ attached to a common carbon are combined to form a C₃₋₆ spirocycloalkyl which is unsubstituted or substituted with 1-3 members independently selected from F, Cl, OH, and CH₃.

In some selected embodiments, the compound of Formula (I) or a pharmaceutically acceptable salt, hydrate, or solvate thereof, including any selected embodiments above, is a compound wherein fused ring B has a formula selected from the group consisting of:

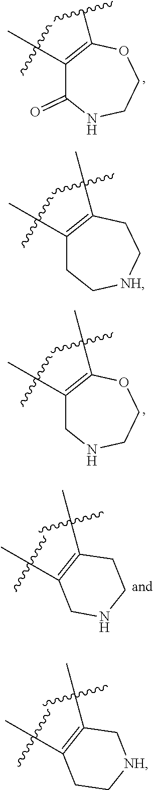 [SMILES: *C1=C(*)C(=O)NCCO1 ; *C1=C(*)CCNCC1 ; *C1=C(*)OCCNC1 ; *C1=C(*)CNCC1 ; *C1=C(*)CNCC1]

each of which is unsubstituted or substituted with 1 to 2 R⁴. In some further selected embodiments, fused ring B is unsubstituted. In other selected embodiments, fused ring B is

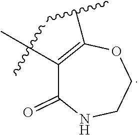 [SMILES: *C1=C(*)C(=O)NCCO1]

In some selected embodiments, the compound of Formula (I) or a pharmaceutically acceptable salt, hydrate, or solvate thereof, including any selected embodiments above, is a compound wherein fused ring B has a formula selected from the group consisting of:

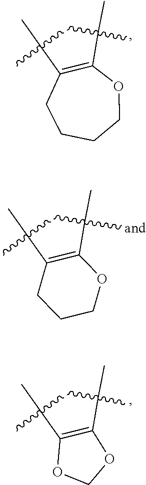

each of which is unsubstituted or substituted with 1 to 4 R⁴. In some further selected embodiments, fused ring B is substituted with 1 to 4 R⁴, each of which is independently selected from the group consisting of halogen, C₁₋₄ alkyl, C₁₋₄ haloalkyl and OH.

In other selected embodiments, the compound of Formula (I) or a pharmaceutically acceptable salt, hydrate, or solvate thereof, including any selected embodiments above, is a compound wherein fused ring B has a formula selected from the group consisting of:

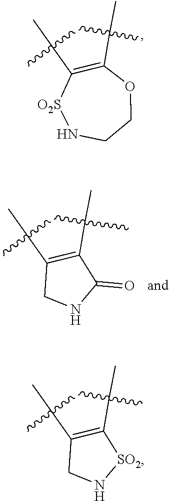

each of which is unsubstituted or substituted with 1 to 3 R⁴. In some further selected embodiments, each R⁴ is independently selected from the group consisting of C₁₋₄ alkyl and C₁₋₄ haloalkyl.

In some selected embodiments, any one compound of Table 1, or a pharmaceutically acceptable salt, solvate or hydrate thereof is provided.

### Methods of Synthesis

### General Methods for the Preparation of Compounds of the Claims

Without limitation, useful methods for constructing the compounds according to this disclosure may consist of four parts, which may be done in any order: connection of the a and b fragments, connection of the b and c fragments, connection of the c and d fragments, or modification of the functional groups present in all fragments. The general retrosynthetic disconnection of the compounds of the disclosure into fragments a-d useful for construction of the compounds is shown below:

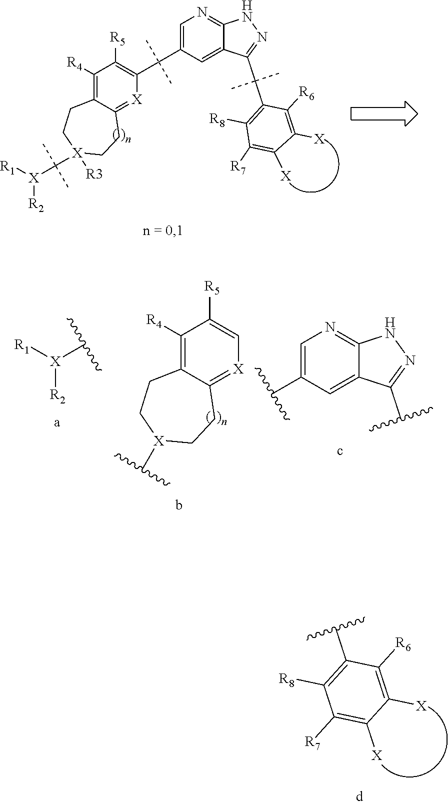

Several methods for the preparation of claimed compounds are exemplary (eq. 1-6). Eq. (1) demonstrates one method of forming the bond between fragments a and b via reductive amination. Formation of the bond between the fragments a and b may take place before or after formation of the bond between the fragments b and c. In the case of Eq. (1), the desired amine is connected to the desired ketone via use of a hydride source and acetic acid or any other conditions known for reductive amination.

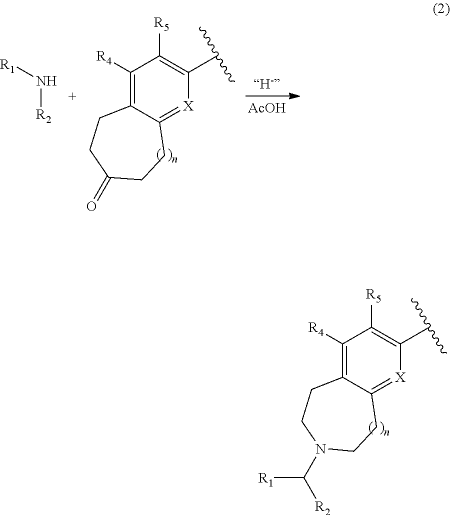

The relative positions of the amine and ketone may be reversed as well, as exemplified in Eq. (2). Those skilled in the art will recognized that there are other possible conditions which will result in the desired connectivity and product.

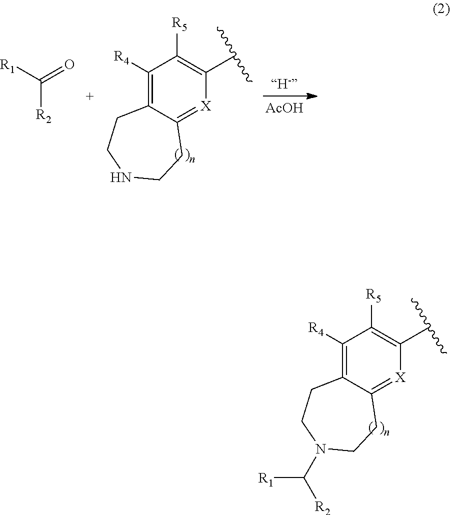

Eq. (3) demonstrates another method of forming the a-b fragment though initial condensation and aminal formation of the two partners, followed by addition of a Grignard reagent. This sequence results in an additional alkyl substituent on the carbon atom adjacent to the amine nitrogen atom.

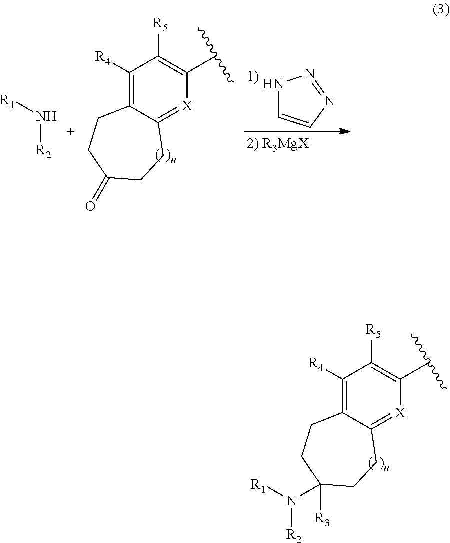

Formation of the bond between the fragments b and c may take place before or after formation of the bond between the fragments a and b or fragments c and d. Eq. (4) demonstrates one method to connect the b and c fragments via cross-coupling. Y may be chosen from an appropriate group such as B(OH)₂, B(OR)₂, ZnCl, MgBr, SnR₃, etc. Z may be chosen from an appropriate group such as Cl, Br, I, OTf, etc. The coupling is mediated by a transition metal catalyst, preferably palladium with an appropriate ligand. The coupling may be assisted by an organic or inorganic base. Use of a protecting group such as SEM, Boc, THP, PMB, MOM, MEM, TIPS, etc. on the bicyclic moiety generally improves the yield and purity of the desired product.

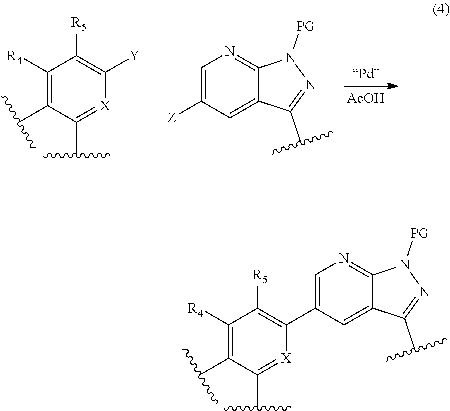

The relative functionalization of the coupling partners may also be reversed, as shown in Eq. (5). Those skilled in the art will recognize there are other possible combinations and conditions which will also result in the desired product.

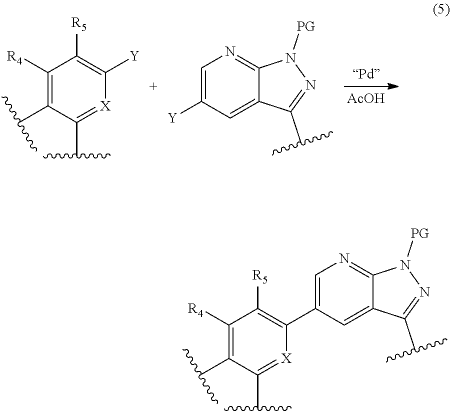

Formation of the bond between the fragments c and d may take place before or after formation of the bond between the fragments b and c. Eq. (6) demonstrates one method to connect the c and d fragments via cross-coupling. Y may be chosen from an appropriate group such as B(OH)₂, B(OR)₂, ZnCl, MgBr, SnR₃, etc. Z may be chosen from an appropriate group such as Cl, Br, I, OTf, etc. The coupling is mediated by a transition metal catalyst, preferably palladium with an appropriate ligand. The coupling may be assisted by an organic or inorganic base. Use of a protecting group such as SEM, Boc, THP, PMB, MOM, MEM, TIPS, etc. on the bicyclic moiety generally improves the yield and purity of the desired product.

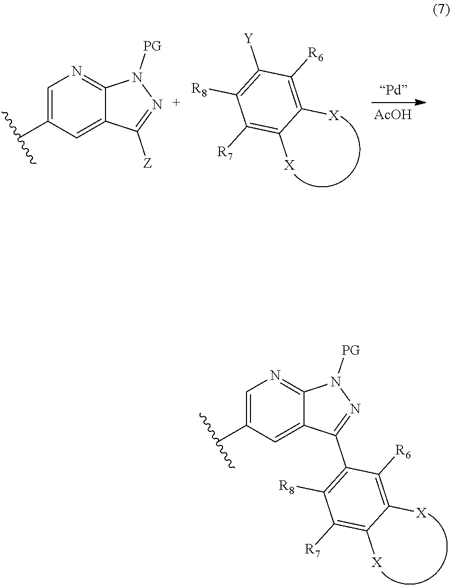

For the most efficient preparation of any particular compound of the disclosure, the timing and order of connection of the fragments and modification of the functionality present in any of the fragments may vary and will be dependent on the functionality present. A variety of the methods described above have been used to prepare compounds of this disclosure, and are exemplified below. Deuterated forms of the below examples can be synthesized using appropriate deuterated intermediates.

### Therapeutic and Prophylactic Uses

The present disclosure contemplates the use of the AXL inhibitors described herein in the treatment or prevention of a range of diseases, disorders and/or conditions, and/or the symptoms thereof. While particular uses are described in detail hereafter, it is to be understood that the present disclosure is not so limited. Furthermore, although general categories of particular diseases, disorders and conditions are set forth hereafter, some of the diseases, disorders and conditions may be a member of more than one category, and others may not be a member of any of the disclosed categories.

In some embodiments, the AXL inhibitors described herein are administered in an amount effective to reverse, stop or slow the progression of AXL-mediated dysregulation.

Oncology-related Disorders. The AXL inhibitors described herein can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), cancers of the hematopoietic system and the immune system (e.g., spleen or thymus), and myelodysplastic syndromes. The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. In particular embodiments, the tumor or cancer is colon cancer, ovarian cancer, breast cancer, bladder cancer (e.g., urothelial carcinoma), oesophageal cancer, kidney cancer (e.g., clear cell renal cell carcinoma), pancreatic cancer (e.g., pancreatic ductal adenocarcinoma), melanoma, liver cancer (e.g., hepatocellular carcinoma), lung cancer (e.g., non-small cell lung carcinoma), head and neck cancer (e.g., head and neck squamous cell carcinoma), glioblastoma, leukemia (e.g., acute myeloid leukemia, and chronic lymphocytic leukemia), or myelodysplastic syndromes. In some embodiments, the cancer is leukemia (e.g., acute myeloid leukemia), lung cancer (e.g., non-small cell lung cancer), or kidney cancer (e.g., clear cell renal cell carcinoma). The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the compounds according to this disclosure are useful in the treatment of kidney cancer. In further embodiments, the kidney cancer is renal cell carcinoma. In still further embodiments, the renal cell carcinoma is clear cell renal carcinoma (ccRCC).

In some embodiments, the compounds according to this disclosure are useful in the treatment of lung cancer. In further embodiments, the lung cancer is non-small cell lung cancer (NSCLC). In still further embodiments, the NSCLC is lung squamous cell carcinoma or lung adenocarcinoma. In some embodiments the NSCLC is EGFR mutant NSCLC.

In some embodiments, the compound according to this disclosure are useful in the treatment of leukemia. In further embodiments, the leukemia is acute myeloid leukemia (AML). In still further embodiments the AML is relapsed AML.

In some embodiments, the compounds according to this disclosure are useful in the treatment of breast cancer. In further embodiments, the breast cancer is hormone receptor positive (e.g., ERα-positive breast cancer, PR-positive breast cancer, ERα-positive and PR-positive breast cancer), HER2 positive breast cancer, HER2 over-expressing breast cancer, or any combination thereof. In still further embodiments, the breast cancer is triple negative breast cancer.

In some embodiments, the compounds according to this disclosure are useful in the treatment of pancreatic cancer. In further embodiments, the pancreatic cancer is pancreatic neuroendocrine tumor or pancreatic adenocarcinoma (i.e., pancreatic ductal adenocarcinoma (PDAC)).

In certain embodiments, a cancer may be metastatic or at risk of becoming metastatic, or may occur in a diffuse tissue, including cancers of the blood or bone marrow (e.g., leukemia, or myelodysplastic syndromes).

Hypoxic conditions of the tumor microenvironment have been shown to upregulate the expression of AXL. Accordingly, in some embodiments, the AXL inhibitors according to the disclosure are useful in treating hypoxic tumors.

In one or more embodiments, the cancer is an oncogene addicted cancer. Oncogene addicted cancers are those that rely on a dominant oncogene for growth and survival, such as, for example, ALK, ABL, AURORA, AKT, PDGFR, KIT, EGFR, VEGF, FGFR3, FLT-3, MYC, RET, BRAF, PI3K, NF-κB, JAK, STAT, BCL-2, MCL-1, KRAS, HRAS, MEK, ERK, HER-2, HER-3 or MET.

In some embodiments, the present disclosure provides methods for treating a proliferative condition, cancer, tumor, or precancerous condition with an AXL inhibitor and at least one additional therapeutic or diagnostic agent, examples of which are set forth elsewhere herein.

Immune- and Inflammatory-related Disorders. A non-limiting list of immune- and inflammatory-related diseases, disorders and conditions which may be treated or prevented with the compounds and compositions of the present disclosure include arthritis (e.g., rheumatoid arthritis), kidney failure, lupus, asthma, psoriasis, colitis, pancreatitis, allergies, fibrosis, surgical complications (e.g., where inflammatory cytokines prevent healing), anemia, and fibromyalgia.

Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (e.g., Crohn's disease and ulcerative colitis), chronic obstructive pulmonary disease (COPD), atherosclerosis, allergic contact dermatitis and other eczemas, systemic sclerosis, transplantation and multiple sclerosis.

In particular embodiments of the present disclosure, the AXL inhibitors are used to increase or enhance an immune response to an antigen by providing adjuvant activity. In a particular embodiment, at least one antigen or vaccine is administered to a subject in combination with at least one AXL inhibitor of the present disclosure to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with at least one AXL inhibitor of the present disclosure.

In some embodiments, an AXL inhibitor described herein can be combined with an immunosuppressive agent to reduce the number of immune effector cells.

Other Disorders. Embodiments of the present disclosure contemplate the administration of the AXL inhibitors described herein to a subject for the treatment or prevention of any other disorder that may benefit from at least some level of AXL inhibition. Such diseases, disorders and conditions include, for example, cardiovascular (e.g., cardiac ischemia) and metabolic (e.g., diabetes, insulin resistance, obesity) disorders.

### Selection of Patients

In some embodiments, patients are selected by assessing AXL expression (e.g., soluble AXL (i.e., sAXL), cell surface AXL, or total AXL) in a relevant tissue or sample. In some embodiments, patients are selected by further assessing GAS6 expression in a relevant tissue or sample. In some embodiments, the disclosure provides a method of treating cancer in a patient having elevated AXL expression with a compound as described herein. In one embodiment, the disclosure provides a method of treating cancer in a patient having elevated cell surface AXL expression with a compound as described herein. In another embodiment, the disclosure provides a method of treating cancer in a patient having elevated sAXL expression with a compound as described herein. In still another embodiment, the disclosure provides a method of treating cancer in a patient having an elevated ratio of sAXL expression to GAS6 expression with a compound as described herein. In some embodiments, the disclosure provides a method of administering a therapeutically effective amount of an AXL inhibitor to an individual for the treatment of cancer based on a determination of the relative amount of AXL expression. In another embodiment, the disclosure provides a method of administering a therapeutically effective amount of an AXL inhibitor to an individual for the treatment of cancer based on a determination of the relative amount of cell surface AXL expression. In another embodiment, the disclosure provides a method of administering a therapeutically effective amount of an AXL inhibitor to an individual for the treatment of cancer based on a determination of the relative amount of sAXL expression. In still another embodiment, the disclosure provides a method of administering a therapeutically effective amount of an AXL inhibitor to an individual for the treatment of cancer based on a determination of the relative ratio of sAXL expression to GAS6 expression.

### Pharmaceutical Compositions

The AXL inhibitors of the present disclosure may be in the form of compositions suitable for administration to a subject. In general, such compositions are “pharmaceutical compositions” comprising an AXL inhibitor(s) as described herein or a pharmaceutically acceptable salt thereof and a pharmaceutically acceptable excipient. In certain embodiments, the AXL inhibitors are present in an effective amount. The pharmaceutical compositions may be used in the methods of the present disclosure

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present disclosure.

The pharmaceutical compositions containing the active ingredient (e.g., an inhibitor of AXL) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared using one or more excipients such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for manufacture. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of an AXL inhibitor contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers that can be included in the pharmaceutical compositions include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector), whereas a multi-use container (e.g., a multi-use vial) is provided in other embodiments.

The pharmaceutical compositions may be in the form of a sterile injectable aqueous or oleagenous suspension. This suspension may be formulated using excipients such as suitable dispersing agents, wetting agents, and/or suspending agents. The sterile injectable preparation may also be a sterile injectable solution or suspension in a non-toxic parenterally-acceptable diluent or solvent as excipient, for example, a solution in 1,3-butane diol. Acceptable diluents, solvents and dispersion media that may be employed as excipients include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils can be employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The AXL inhibitors contemplated by the present disclosure may be in the form of any other suitable pharmaceutical composition (e.g., sprays for nasal or inhalation use) currently known or developed in the future.

### Routes of Administration

The present disclosure contemplates the administration of AXL inhibitors, and compositions thereof, in any appropriate manner. Suitable routes of administration include oral, parenteral (e.g., intramuscular, intravenous, subcutaneous (e.g., injection or implant), intraperitoneal, intracisternal, intraarticular, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the AXL inhibitors disclosed herein over a defined period of time.

Particular embodiments of the present disclosure contemplate oral administration.

### Combination Therapy

The present disclosure contemplates the use of AXL inhibitors alone or in combination with one or more active therapeutic agents. The additional active therapeutic agents can be small chemical molecules; macromolecules such as proteins, antibodies, peptibodies, peptides, DNA, RNA or fragments of such macromolecules; or cellular or gene therapies. The combination therapy may target different, but complementary mechanisms of action and thereby have a synergistic therapeutic or prophylactic effect on the underlying disease, disorder, or condition.

In addition or alternatively, the combination therapy may allow for a dose reduction of one or more of the agents, thereby ameliorating, reducing or eliminating adverse effects associated with one or more of the agents.

The active therapeutic agents in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

In some embodiments, the AXL inhibitor according to this disclosure is combined with at least one additional therapeutic agent. In some embodiments, the at least one additional therapeutic agent comprises one or more agents independently selected from the groups consisting of inhibitors of the CD47-SIRPα pathway (e.g., anti-CD47 antibodies), inhibitors of HIF (e.g., a HIF-2α inhibitor), immune checkpoint inhibitors, agents that targets the extracellular production of adenosine (e.g., CD73 inhibitors, CD39 inhibitors, and/or adenosine receptor inhibitors (e.g., A_{2A}R and/or A_{2B}R inhibitors), radiation therapy, and chemotherapeutic agents. Each of the additional therapeutic agents are described in further detail below.

In some embodiments, one or more of the additional therapeutic agents is an immunomodulatory agent. Suitable immunomodulatory agents contemplated by the present disclosure include CD40L, B7, and B7RP1; activating monoclonal antibodies (mAbs) to stimulatory receptors, such as, anti-CD40, anti-CD38, anti-ICOS, and 4-IBB ligand; dendritic cell antigen loading (in vitro or in vivo); anti-cancer vaccines such as dendritic cell cancer vaccines; cytokines/chemokines, such as, IL1, IL2, IL12, IL18, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); indoleamine 2,3-dioxygenase 1 (IDO1) inhibitors and immune-stimulatory oligonucleotides.

In certain embodiments, the present disclosure provides methods for tumor suppression of tumor growth comprising administration of an AXL inhibitor described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Signal transduction inhibitors (STIs) contemplated by the present disclosure include: (i) BCR-ABL kinase inhibitors (e.g., GLEEVEC®); (ii) epidermal growth factor receptor tyrosine kinase inhibitors (EGFR TKIs), including small molecule inhibitors (e.g., gefitinib, erlotinib, afatinib, and osimertinib), and anti-EGFR antibodies; (iii) inhibitors of the human epidermal growth factor (HER) family of transmembrane tyrosine kinases, e.g., HER-2/neu receptor inhibitors (e.g., HERCEPTIN®), and HER-3 receptor inhibitors; (iv) vascular endothelial growth factor receptor (VEGFR) inhibitors including small molecule inhibitors (e.g., axitinib, sunitinib and sorafenib), and anti-VEGF antibodies (e.g., bevacizumab); (v) inhibitors of AKT family kinases or the AKT pathway (e.g., rapamycin); (vi) inhibitors of serine/threonine-protein kinase B-Raf (BRAF), such as, for example, vemurafenib, dabrafenib and encorafenib; (vii) inhibitors of rearranged during transfection (RET), including, for example, selpercatinib and pralsetinib; (viii) tyrosine-protein kinase Met (MET) inhibitors (e.g., tepotinib, tivantinib, cabozantinib, pazopanib, tivozanib, XL-092, and crizotinib); (ix) anaplastic lymphoma kinase (ALK) inhibitors (e.g., ensartinib, ceritinib, lorlatinib, crizotinib, and brigatinib); (x) inhibitors of the RAS signaling pathway (e.g., inhibitors of KRAS, HRAS, RAF, MEK, ERK) as described elsewhere herein; (xi) FLT-3 inhibitors (e.g., gilteritinib); (xii) inhibitors of Trop-2; (xiii) inhibitors of the JAK/STAT pathway, e.g., JAK inhibitors including tofacitinib and ruxolitinib, or STAT inhibitors such as napabucasin; (xiv) inhibitors of NF-κB; (xv) cell cycle kinase inhibitors (e.g., flavopiridol); (xvi) phosphatidyl inositol kinase (PI3K) inhibitors; and (xvii) protein kinase B (AKT) inhibitors (e.g., capivasertib, miransertib). Agents involved in immunomodulation can also be used in combination with the AXL inhibitors described herein for the suppression of tumor growth in cancer patients. In one or more embodiments, the additional therapeutic agent comprises an inhibitor of EGFR, VEGFR, HER-2, HER-3, BRAY, RET, MET, ALK, RAS (e.g., KRAS, MEK, ERK), FLT-3, JAK, STAT, NF-κB, PI3K, AKT, BCL-2, MCL-1, CD47, or any combinations thereof.

In some embodiments, one or more of the additional therapeutic agents comprise a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; anthracyclines; and pharmaceutically acceptable salts, acids or derivatives of any of the above. In some embodiments, the chemotherapeutic agent is a platinum-based, anthracycline-based, or taxoid-based chemotherapeutic agent. In some embodiments, the chemotherapeutic agent is cisplatin, carboplatin, oxaliplatin, doxorubicin, docetaxel or paclitaxel.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as abiraterone, enzalutamide, apalutamide, darolutamide, flutamide, nilutamide, bicalutamide, leuprolide, and goserelin; and pharmaceutically acceptable salts, acids or derivatives of any of the above. In certain embodiments, combination therapy comprises a chemotherapy regimen that includes one or more chemotherapeutic agents. In certain embodiments, combination therapy comprises administration of a hormone or related hormonal agent.

Combinations of the AXL inhibitor according to this disclosure with a poly (ADP-ribose) polymerase (PARP) inhibitor is also contemplated. Exemplary PARP inhibitors contemplated by this disclosure include olaparib, niraparib and rucaparib.

Additional treatment modalities that may be used in combination with an AXL inhibitor include radiotherapy, a monoclonal antibody against a tumor antigen, a complex of a monoclonal antibody and toxin, a T-cell adjuvant, bone marrow transplant, or antigen presenting cells (e.g., dendritic cell therapy), including TLR agonists which are used to stimulate such antigen presenting cells.

In certain embodiments, the present disclosure contemplates the use of the compounds described herein in combination with adoptive cell therapy, a new and promising form of personalized immunotherapy in which immune cells with anti-tumor activity are administered to cancer patients. Adoptive cell therapy is being explored using tumor-infiltrating lymphocytes (TIL) and T cells engineered to express, for example, chimeric antigen receptors (CAR) or T cell receptors (TCR). Adoptive cell therapy generally involves collecting T cells from an individual, genetically modifying them to target a specific antigen or to enhance their anti-tumor effects, amplifying them to a sufficient number, and infusion of the genetically modified T cells into a cancer patient. T cells can be collected from the patient to whom the expanded cells are later reinfused (e.g., autologous) or can be collected from donor patients (e.g., allogeneic).

In certain embodiments, the present disclosure contemplates the use of the compounds described herein in combination with RNA interference-based therapies to silence gene expression. RNAi begins with the cleavage of longer double-stranded RNAs into small interfering RNAs (siRNAs). One strand of the siRNA is incorporated into a ribonucleoprotein complex known as the RNA-induced silencing complex (RISC), which is then used to identify mRNA molecules that are at least partially complementary to the incorporated siRNA strand. RISC can bind to or cleave the mRNA, both of which inhibits translation.

In certain embodiments, the present disclosure contemplates the use of the compounds described herein in combination with agents that target the extracellular production of adenosine. Such therapeutic agents may act on the ectonucleotidases that catalyze the conversion of ATP to adenosine, including ectonucleoside triphosphate diphosphohydrolase 1 (ENTPD1, also known as CD39 or Cluster of Differentiation 39), which hydrolyzes ATP to ADP and ADP to AMP, and ecto-5′-nucleotidase (NT5E or 5NT, also known as CD73 or Cluster of Differentiation 73), which converts AMP to adenosine. The enzymatic activities of CD39 and CD73 play strategic roles in calibrating the duration, magnitude, and chemical nature of purinergic signals delivered to various cells (e.g., immune cells). Alteration of these enzymatic activities can change the course or dictate the outcome of several pathophysiological events, including cancer, autoimmune diseases, infections, atherosclerosis, and ischemia-reperfusion injury, suggesting that these ecto-enzymes represent novel therapeutic targets for managing a variety of disorders. Exemplary anti-CD39 and anti-CD73 antibodies include ES002023, TTX-030, IPH-5201, SRF-617, CPI-006, oleclumab (MEDI9447), NZV930, IPH5301, uliledlimab (TJD5, TJ004309), and BMS-986179. In one or more embodiments, the present disclosure contemplates combination with CD73 inhibitors such as those described in WO 2017/120508, WO 2018/094148, WO 2018/067424, and WO 2020/046813. In one embodiment, the CD73 inhibitor is quemliclustat (AB680).

Another approach to targeting the extracellular production of adenosine is to target adenosine A_{2A} and/or A_{2B} receptors. Thus, in some embodiments, this disclosure contemplates the combination of the compounds according to this disclosure with agents that target A_{2A} and/or A_{2B} receptors. Such therapeutic agents can be adenosine 2 receptor (A₂R) (e.g., A_{2A} and/or A_{2B}) antagonists. Adenosine can bind to and activate four different G-protein coupled receptors: A₁R, A_{2A}R, A_{2B}R, and A₃R. The binding of adenosine to the A_{2A}R receptor, which is expressed on T cells, natural killer cells and myeloid cells such as dendritic cells, leads to increased intracellular levels of cyclic AMP and the impairment of maturation and/or activation of such cells. This process significantly impairs the activation of the immune system against cancer cells. In addition, A_{2A}R has been implicated in selectively enhancing anti-inflammatory cytokines, promoting the upregulation of PD-1 and CTLA-4, promoting the generation of LAG-3 and Foxp3+ regulatory T cells, and mediating the inhibition of regulatory T cells. PD-1, CTLA-4 and other immune checkpoints are discussed further herein. Combining A₂R antagonists in the combinations described herein may provide at least an additive effect in view of their differing mechanisms of actions. In one embodiment, the therapeutic agent can be an adenosine receptor antagonist as described in WO/2018/136700, WO 2018/204661, or WO 2020/023846. In one embodiment, the adenosine receptor antagonist is AB928 (i.e., etrumadenant).

In certain embodiments, the present disclosure contemplates the use of the compounds described herein in combination with inhibitors of phosphatidylinositol 3-kinases (PI3Ks), particularly the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T-cell responses leading to decreased cancer development and spread. PI3Kγ inhibitors include those described in WO 2020/0247496A1.

In certain embodiments, the present disclosure contemplates the use of the compounds described herein in combination with inhibitors of arginase, which has been shown to be either responsible for or to participate in inflammation-triggered immune dysfunction, tumor immune escape, immunosuppression and immunopathology of infectious disease. Exemplary arginase compounds can be found, for example, in PCT/US2019/020507 and WO 2020/102646.

In certain embodiments, the present invention contemplates the use of the AXL inhibitors according to this disclosure with inhibitors of HIF-2a, which plays an integral role in cellular response to low oxygen availability. Under hypoxic conditions, the hypoxia-inducible factor (HIF) transcription factors can activate the expression of genes that regulate metabolism, angiogenesis, cell proliferation and survival, immune evasion, and inflammatory response. HIF-2a overexpression has been associated with poor clinical outcomes in patients with various cancers; hypoxia is also prevalent in many acute and chronic inflammatory disorders, such as inflammatory bowel disease and rheumatoid arthritis. Exemplary HIF-2α inhibitors include belzutifan, ARO-HIF2, PT-2385, AB521, and those described in WO 2021113436 and WO 2021188769. In some embodiments, the AXL inhibitors according to this disclosure are combined with AB521.

The present disclosure also contemplates the combination of the AXL inhibitors described herein with one or more RAS signaling inhibitors. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTOR (e.g., mTORC1), SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), MRTX849, mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTOR inhibitors, SHP2 inhibitors, and AKT inhibitors. In other embodiments the one or more RAS signaling inhibitors directly inhibit RAS mutants.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., an anti-CD47 antibody) (v) an isocitrate dehydrogenase (IDH) inhibitor, e.g., IDH-1 or IDH-2 inhibitor (e.g., ivosidenib, enasidenib, etc.).

Immune Checkpoint Inhibitors. The present disclosure contemplates the use of the inhibitors of AXL described herein in combination with immune checkpoint inhibitors.

The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism.

T-cells have been the major focus of efforts to therapeutically manipulate endogenous antitumor immunity because of i) their capacity for the selective recognition of peptides derived from proteins in all cellular compartments; ii) their capacity to directly recognize and kill antigen-expressing cells (by CD8+ effector T cells; also known as cytotoxic T lymphocytes (CTLs)); and iii) their ability to orchestrate diverse immune responses by CD4+ helper T cells, which integrate adaptive and innate effector mechanisms.

In the clinical setting, the blockade of immune checkpoints—which results in the amplification of antigen-specific T cell responses—has shown to be a promising approach in human cancer therapeutics.

T cell-mediated immunity includes multiple sequential steps, each of which is regulated by counterbalancing stimulatory and inhibitory signals in order to optimize the response. While nearly all inhibitory signals in the immune response ultimately modulate intracellular signaling pathways, many are initiated through membrane receptors, the ligands of which are either membrane-bound or soluble (cytokines). While co-stimulatory and inhibitory receptors and ligands that regulate T-cell activation are frequently not over-expressed in cancers relative to normal tissues, inhibitory ligands and receptors that regulate T cell effector functions in tissues are commonly overexpressed on tumor cells or on non-transformed cells associated with the tumor microenvironment. The functions of the soluble and membrane-bound receptor ligand immune checkpoints can be modulated using agonist antibodies (for co-stimulatory pathways) or antagonist antibodies (for inhibitory pathways). Thus, in contrast to most antibodies currently approved for cancer therapy, antibodies that block immune checkpoints do not target tumor cells directly, but rather target lymphocyte receptors or their ligands in order to enhance endogenous antitumor activity. [See Pardoll, (April 2012) Nature Rev. Cancer 12:252-64].

Examples of immune checkpoints (ligands and receptors), some of which are selectively upregulated in various types of tumor cells, that are candidates for blockade include PD-1 (programmed cell death protein 1); PD-L1 (PD-1 ligand); BTLA (B and T lymphocyte attenuator); CTLA-4 (cytotoxic T-lymphocyte associated antigen 4); TIM-3 (T-cell membrane protein 3); LAG-3 (lymphocyte activation gene 3); TIGIT (T cell immunoreceptor with Ig and ITIM domains); and Killer Inhibitory Receptors, which can be divided into two classes based on their structural features: i) killer cell immunoglobulin-like receptors (KIRs), and ii) C-type lectin receptors (members of the type II transmembrane receptor family). Other less well-defined immune checkpoints have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

The present disclosure contemplates the use of the inhibitors of AXL described herein in combination with inhibitors of the aforementioned immune-checkpoint receptors and ligands, as well as yet-to-be-described immune-checkpoint receptors and ligands. Certain modulators of immune checkpoints are currently approved, and many others are in development. When it was approved for the treatment of melanoma in 2011, the fully humanized CTLA-4 monoclonal antibody ipilimumab (YERVOY®; Bristol-Myers Squibb) became the first immune checkpoint inhibitor to receive regulatory approval in the US. Fusion proteins comprising CTLA-4 and an antibody (CTLA4-Ig; abatcept (ORENCIA®; Bristol-Myers Squibb)) have been used for the treatment of rheumatoid arthritis, and other fusion proteins have been shown to be effective in renal transplantation patients that are sensitized to Epstein Barr Virus. The next class of immune checkpoint inhibitors to receive regulatory approval were against PD-1 and its ligands PD-L1 and PD-L2. Approved anti-PD-1 antibodies include nivolumab (OPDIVO®; Bristol-Myers Squibb) and pembrolizumab (KEYTRUDA®; Merck) for various cancers, including squamous cell carcinoma, classical Hodgkin lymphoma and urothelial carcinoma. Approved anti-PD-L1 antibodies include avelumab (BAVENCIO®, EMID Serono & Pfizer), atezolizumab (TECENTRIQ®; Roche/Genentech), and durvalumab (IMFINZI®; AstraZeneca) for certain cancers, including urothelial carcinoma. Another approach to target the PD-1 receptor is the recombinant protein composed of the extracellular domain of PD-L2 (B7-DC) fused to the Fc portion of IgG1, called AMP-224. While there are no approved therapeutics targeting TIGIT or its ligands CD155 and CD112, those in development include BMS-986207 (Bristol-Myers Squibb), tiragolumab (Roche/Genentech), OMP-31M32 (OncoMed), etigilimab, ociperlimab, vibostolimab, AB308, and AB154 (domvanalimab).

In some embodiments, one or more of the additional therapeutic agents is an immuno-oncology agent (e.g., an immune checkpoint inhibitor). In some embodiments, the immune-oncology agent is a PD-1 antagonist, such as an antagonistic PD-1 antibody. Suitable PD-1 antibodies include, for example, OPDIVO® (nivolumab), KEYTRUDA® (pembrolizumab), MEDI-0680 (AMP-514; WO2012/145493), balstilimab, budigalimab, camrelizumab, cemiplimab, dostarlimab, emiplimab, ezabenlimab, pimivalimab, retifanlimab, sasanlimab, spartalizumab, sintilmab, tislelizumab, toripalimab or zimberelimab. The immuno-oncology agent may also include pidilizumab (CT-011), though its specificity for PD-1 binding has been questioned.

In some embodiments, immuno-oncology agent targets PD-L1 and is a PD-L1 antagonist, such as an antagonistic PD-L1 antibody. Suitable PD-L1 antibodies include, for example, TECENTRIQ® (atezolizumab; MPDL3280A; WO2010/077634), IMFINZI® (durvalumab, MEDI4736), BMS-936559 (WO2007/005874), cosibelimab, envafolimab, and avelumab (MSB0010718C; WO2013/79174).

In some combinations provided herein, the compounds according to this disclosure are combined with one or more immune checkpoint inhibitors selected from MEDI-0608, nivolumab, pidilizumab, pembrolizumab, avelumab, atezolizumab, durvalumab, cemiplimab sentilimab, tislelizumab, AB308, domvanalimab, and zimberelimab.

In one aspect of the present disclosure, the claimed AXL inhibitors are combined with an immuno-oncology agent that is (i) an agonist of a stimulatory (including a co-stimulatory) receptor or (ii) an antagonist of an inhibitory (including a co-inhibitory) signal on T cells, both of which result in amplifying antigen-specific T cell responses. Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), B7-H6, and B7-H7 (HHLA2). Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fni4, TWEAK, BAFFR, EDAR, XEDAR, TACI, APRIL, BCMA, LT13R, LIGHT, DcR3, HVEM, VEGI/TL1A, TRAMP/DR3, EDAR, EDA1, XEDAR, EDA2, TNFR1, Lymphotoxin a/TNF13, TNFR2, TNFa, LT13R, Lymphotoxin a 1132, FAS, FASL, RELT, DR6, TROY, NGFR.

In another aspect, the immuno-oncology agent is a cytokine that inhibits T cell activation (e.g., IL-6, IL-10, TGF-B, VEGF, and other immunosuppressive cytokines) or a cytokine that stimulates T cell activation, for stimulating an immune response.

In one aspect, T cell responses can be stimulated by a combination of the disclosed AXL inhibitors and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and/or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD2. Other agents that can be combined with the AXL inhibitors of the present disclosure for the treatment of cancer include antagonists of inhibitory receptors on NK cells or agonists of activating receptors on NK cells. For example, compounds herein can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In another aspect, the disclosed AXL inhibitors can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY® (ipilimumab) or tremelimumab.

In another aspect, the immuno-oncology agent is a PD-1 antagonist, such as those described elsewhere herein.

In another aspect, the immuno-oncology agent is a PD-L1 antagonist, such as those described elsewhere herein.

In another aspect, the immuno-oncology agent is a TIGIT antagonist, such as those described elsewhere herein.

In another aspect, the immuno-oncology agent is a LAG-3 antagonist, such as an antagonistic LAG-3 antibody. Suitable LAG-3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO11/109400).

Examples of therapeutic agents useful in combination therapy for the treatment of cardiovascular and/or metabolic-related diseases, disorders and conditions include statins (e.g., CRESTOR®, LESCOL®, LIPITOR®, MEVACOR®, PRAVACOL®, and ZOCOR®), which inhibit the enzymatic synthesis of cholesterol; bile acid resins (e.g., COLESTID, LO-CHOLEST, PREVALITE®, QUESTRAN®, and WELCHOL®), which sequester cholesterol and prevent its absorption; ezetimibe (ZETIA®), which blocks cholesterol absorption; fibric acid (e.g., TRICOR®), which reduces triglycerides and may modestly increase HDL; niacin (e.g., NIACOR®), which modestly lowers LDL cholesterol and triglycerides; and/or a combination of the aforementioned (e.g., VYTORIN (ezetimibe with simvastatin)). Alternative cholesterol treatments that may be candidates for use in combination with the AXL inhibitors described herein include various supplements and herbs (e.g., garlic, policosanol, and guggul).

Examples of therapeutic agents useful in combination therapy for immune- and inflammatory-related diseases, disorders or conditions include, but are not limited to, the following: non-steroidal anti-inflammatory drug (NSAID) such as aspirin, ibuprofen, and other propionic acid derivatives (alminoprofen, benoxaprofen, bucloxic acid, carprofen, fenbufen, fenoprofen, fluprofen, flurbiprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone). Other combinations include cyclooxygenase-2 (COX-2) inhibitors.

Other active agents for combination include steroids such as prednisolone, prednisone, methylprednisolone, betamethasone, dexamethasone, or hydrocortisone. Such a combination may be especially advantageous since one or more adverse effects of the steroid can be reduced or even eliminated by tapering the steroid dose required.

Additional examples of active agents that may be used in combinations for treating, for example, rheumatoid arthritis, include cytokine suppressive anti-inflammatory drug(s) (CSAIDs); antibodies to, or antagonists of, other human cytokines or growth factors, for example, TNF, LT, IL-10, IL-2, IL-6, IL-7, IL-8, IL-15, IL-16, IL-18, EMAP-II, GM-CSF, FGF, or PDGF.

Particular combinations of active agents may interfere at different points in the autoimmune and subsequent inflammatory cascade, and include TNF antagonists such as chimeric, humanized or human TNF antibodies, REMICADE®, HUMIRA®, anti-TNF antibody fragments (e.g., CDP870), and soluble p55 or p75 TNF receptors, derivatives thereof, p75TNFRIgG (ENBREL®) or p55TNFR1gG (LENERCEPT), soluble IL-13 receptor (sIL-13), and also TNFa-converting enzyme (TACE) inhibitors; similarly, IL-1 inhibitors (e.g., Interleukin-1-converting enzyme inhibitors) may be effective. Other combinations include Interleukin 11, anti-P7s and p-selectin glycoprotein ligand (PSGL). Other examples of agents useful in combination with the AXL inhibitors described herein include interferon-131a (AVONEX®); interferon-131b (BETASERON®); copaxone; hyperbaric oxygen; intravenous immunoglobulin; clabribine; and antibodies to, or antagonists of, other human cytokines or growth factors (e.g., antibodies to CD40 ligand and CD80).

In one or more embodiments, combinations of the AXL inhibitors according to this disclosure with DNA methyltransferase (DNMT) inhibitors or hypomethylating agents is also contemplated. Exemplary DNMT inhibitors include decitabine, zebularine and azacitadine.

In one or more embodiments, combinations of the AXL inhibitors according to this disclosure with a histone deacetylase (HDAC) inhibitor is also contemplated. Exemplary HDAC inhibitors include vorinostat, givinostat, abexinostat, panobinostat, belinostat and trichostatin A.

In some embodiments, the AXL inhibitors according to this disclosure are combined with a menin-MLL inhibitor.

In some embodiments, combination of the AXL inhibitors according to this disclosure with a isocitrate dehydrogenase (IDH) inhibitor, e.g., IDH-1 or IDH-2, is also contemplated. An exemplary IDH-1 inhibitor is ivosidenib. An exemplary IDH-2 inhibitor is enasidenib.

The present disclosure encompasses pharmaceutically acceptable salts, acids or derivatives of any of the above.

Selection of the additional therapeutic agent(s) may be informed by current standard of care for a particular cancer and/or mutational status of a subject's cancer and/or stage of disease.

Detailed standard of care guidelines are published, for example, by National Comprehensive Cancer Network (NCCN). See, for instance, NCCN Acute Myeloid Leukemia v1.2022, NCCN Acute Lymphoblastic Leukemia v1.2022, NCCN Multiple Myeloma v5.2022, NCCN Non-Small Cell Lung Cancer v3.2022, NCCN Kidney Cancer v4.2022, NCCN Colon Cancer v1.2022, NCCN Rectal Cancer v1.2022, NCCN Hepatobiliary Cancer v1.2022, NCCN Pancreatic Adenocarcinoma v1.2022, NCCN Esophageal and Esophagogastric Junction Cancers v2.2022, NCCN Prostate Cancer v3.2022, NCCN Gastric Cancer v2.2022, Cervical Cancer v1.2022, Ovarian Cancer/Fallopian Tube Cancer/Primary Peritoneal Cancer v1.2022, NCCN Breast Cancer v2.2022.

### Dosing

The AXL inhibitors of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered and prior or concomitant therapy. Effective dosage amounts and dosage regimens can be determined from, for example, safety and dose-escalation trials, in vivo studies (e.g., animal models).

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

In general, the disclosed methods comprise administering a compound described herein, or a pharmaceutically acceptable salt or solvate thereof, or a composition thereof, in an effective amount to a subject in need thereof. An “effective amount” with reference to an AXL inhibitor of the present disclosure means an amount of the compound that is sufficient to engage the target (by inhibiting, agonizing or antagonizing the target) at a level that is indicative of the potency of the compound. For AXL, target engagement can be determined by one or more biochemical or cellular assays resulting in an EC50, ED50, EC90, IC50, or similar value which can be used as one assessment of the potency of the compound. Assays for determining target engagement include, but are not limited to, those described in the Examples. The effective amount may be administered as a single quantity or as multiple, smaller quantities (e.g., as one tablet with “x” amount, as two tablets each with “x/2” amount, etc.).

In certain embodiments, the AXL inhibitors contemplated by the present disclosure may be administered (e.g., orally, parenterally, etc.) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1 to 1000 milligrams of the active ingredient (i.e. a compound of Formula (I), particularly 1, 3, 5, 10, 15, 20, 25, 50, 75, 100, 150, 200, 250, 300, 400, 500, 600, 750, 800, 900, and 1000 milligrams of the active ingredient.

In certain embodiments, the dosage of the desired AXL inhibitor is contained in a “unit dosage form”. The phrase “unit dosage form” refers to physically discrete units, each unit containing a predetermined amount of the AXL inhibitor, either alone or in combination with one or more additional agents, sufficient to produce the desired effect. It will be appreciated that the parameters of a unit dosage form will depend on the particular agent and the effect to be achieved. For intravenous administration, the unit dosage form may contain from 1 to 1000 milligrams of the active ingredient (i.e. a compound of Formula (I), particularly 1, 10, 25, 50, 100, 200, 300, or 500 milligrams.

### Kits

The present disclosure also contemplates kits comprising a compound described herein, and pharmaceutical compositions thereof. The kits are generally in the form of a physical structure housing various components, as described below, and may be utilized, for example, in practicing the methods described above.

A kit can include one or more of the compounds disclosed herein (provided in, e.g., a sterile container), which may be in the form of a pharmaceutical composition suitable for administration to a subject. The compounds described herein can be provided in a form that is ready for use (e.g., a tablet or capsule) or in a form requiring, for example, reconstitution or dilution (e.g., a powder) prior to administration. When the compounds described herein are in a form that needs to be reconstituted or diluted by a user, the kit may also include diluents (e.g., sterile water), buffers, pharmaceutically acceptable excipients, and the like, packaged with or separately from the compounds described herein. When combination therapy is contemplated, the kit may contain the several agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium. In some embodiments, the actual instructions are not present in the kit, but means for obtaining the instructions from a remote source, e.g., via the internet, are provided.

### EXPERIMENTAL

The following examples are put forth so as to provide those of ordinary skill in the art with a complete disclosure and description of how to make and use the present disclosure, and are not intended to limit the scope of what the inventors regard as their invention. Efforts have been made to ensure accuracy with respect to numbers used (e.g., amounts, temperature, etc.), but some experimental errors and deviations should be accounted for.

Unless indicated otherwise, temperature is in degrees Celsius (° C.), and pressure is at or near atmospheric. Standard abbreviations are used, including the following: rt or r.t.=room temperature; min=minute(s); h or hr-hour(s); ng=nanogram; gg=microgram; mg=milligram; g=gram; kg=kilogram; μl or μL=microliter; ml or mL=milliliter; 1 or L=liter; gM=micromolar; mM=millimolar; M=molar; mol=mole; mmol=millimole; aq.=aqueous; calcd=calculated; DCM=dichloromethane; DCE=1,2-dichloroethane; MTBE=methyl tert-butyl ether; THF=tetrahydrofuran; EtOAc=ethyl acetate; ACN=acetonitrile; NMP=N-methyl-2-pyrrolidone; DMF=N,N-dimethylformamide; DMSO=dimethyl sulfoxide; IPA=isopropanol; EtOH=ethanol; MeOH=methanol; H₂=hydrogen gas; N₂=nitrogen gas; DIPEA=N,N-diisopropylethylamine; DMEDA=N,N-dimethylethane-1,2-diamine; HATU=N-[(Dimethylamino)-1H-1,2,3-triazolo-[4,5-b]pyridin-1-ylmethylene]-N-methylmethanaminium hexafluorophosphate N-oxide; EDC=1-ethyl-3-(3-dimethylaminopropyl)carbodiimide; HOBt=hydroxybenzotriazole; NBS═N-bromosuccinimide; KOAc=potassium acetate; TFA=trifluoroacetic acid; (dppf)PdCl₂=[1,1′-bis(diphenylphosphino)ferrocene]palladium(II) dichloride; B₂pin₂=bis(pinacolato)diboron; DMAP=4-dimethylaminopyridine; MHz=megahertz; Hz=hertz; ppm=parts per million; ESI MS-electrospray ionization mass spectrometry; NMR=nuclear magnetic resonance.

### Materials and Methods

The following general materials and methods were used, where indicated, or may be used in the Examples below:

¹H NMR spectra were recorded on a Varian 400 MHz NMR spectrometer equipped with an Oxford AS400 magnet. Chemical shifts (δ) are reported as parts per million (ppm) relative to residual undeuterated solvent as an internal reference.

### EXAMPLES

### Example 1: 8-{5-[7-(Pyrrolidin-1-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

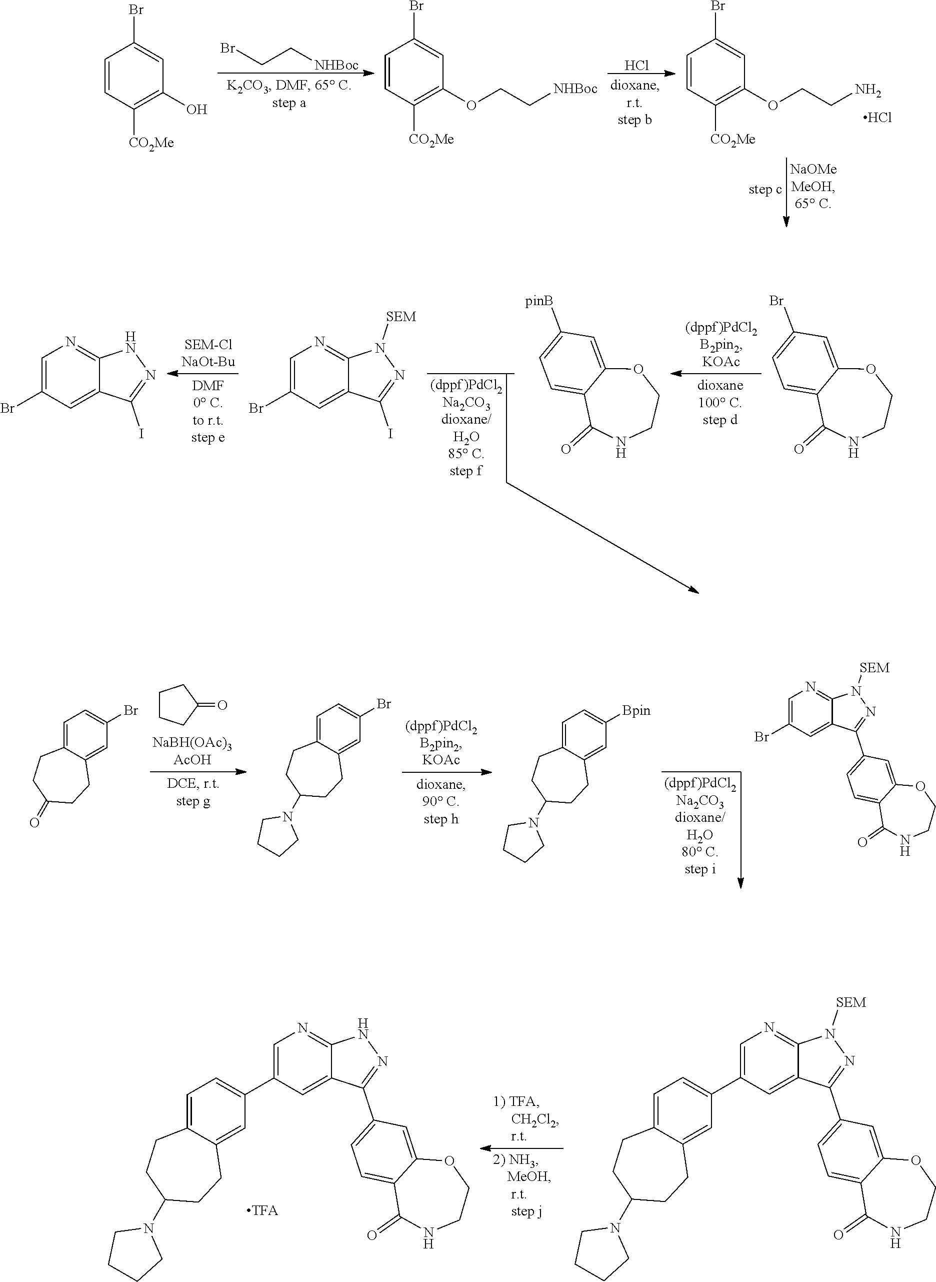

Step a: To a mixture of methyl 4-bromo-2-hydroxybenzoate (4.62 g, 20.0 mmol), K₂CO₃ (5.53 g, 40.0 mmol), and DMF (40 mL) at r.t. was added tert-butyl (2-bromoethyl)carbamate (4.71 g, 21.0 mmol). The reaction mixture was stirred at 65° C. for 3 hours, cooled to r.t., diluted with EtOAc (200 mL), washed with 9:1 water:brine (4×200 mL), dried over Na₂SO₄, and concentrated. The crude material was purified by column chromatography (120 g silica gel, hexanes:EtOAc) 0% to 50% gradient (25 minutes) to afford the desired product as a light yellow oil (7.02 g; 94%).

Step b: A mixture of the product from step a (7.02 g, 18.8 mmol) and 4M HCl in dioxane (38 mL) was stirred at r.t. for 30 minutes and diluted with MTBE (300 mL). The precipitated solids were collected by filtration, washed with MTBE, and dried to afford the desired product as a white solid (5.07 g; 87%).

Step c: To a mixture of the product from step b (5.07 g, 16.3 mmol) and MeOH (41 mL) at r.t. was added NaOMe (7.47 mL, 32.6 mmol, 25% wt. in MeOH). The reaction mixture was stirred at 65° C. for 1 hour, cooled to r.t., quenched with sat. NH₄Cl_{(aq.)} (7.5 mL), and diluted with EtOAc (150 mL). The organic phase was washed with water (1×100 mL), dried over Na₂SO₄, and concentrated. The crude material was purified by column chromatography (80 g silica gel, CH₂Cl₂:MeOH) 0% to 10% gradient (30 minutes) to afford the desired product as a white solid (3.82 g; 97%).

Step d: A mixture of the product from step c (1.21 g, 5.00 mmol), B₂pin₂ (1.27 g, 5.00 mmol), (dppf)PdCl₂ (183 mg, 0.250 mmol), and KOAc (981 mg, 10.0 mmol) was placed under nitrogen. Degassed dioxane (25 mL) was added and the reaction mixture was stirred at 100° C. for 1 hour. The mixture was cooled to r.t., concentrated, diluted with EtOAc (250 mL), filtered through celite to remove solids, and again concentrated to afford the desired product which was used crude in the next step.

Step e: To a suspension of 5-bromo-3-iodo-1H-pyrazolo[3,4-b]pyridine (40.3 g, 124 mmol) in DMF (124 mL) at 0° C. was added solid NaOt-Bu (14.6 g, 130 mmol) in 3 portions over −20 min and then the mixture stirred for an additional 10 min. (2-(Chloromethoxy)ethyl)trimethylsilane (23.0 mL, 130 mmol) was added over 30 minutes and then the reaction was stirred for 15 h, warming to r.t. as the cooling bath expired. The mixture was cooled to 0° C. and diluted with H₂O (500 mL). The precipitated solids were collected by filtration, washed with H₂O, and dried in vacuo to afford the desired product as a light yellow solid (51.2 g; 91%).

Step f: To a mixture of the product of step e (4.76 g, 10.5 mmol), K₂CO₃ (2.90 g, 21.0 mmol), and (dppf)PdCl₂ (766 mg, 1.05 mmol) under N₂ was added a solution of the crude product of step d (10.5 mmol) in degassed dioxane (48 mL) followed by degassed H₂O (12 mL). The reaction mixture was stirred at 85° C. for 20 h, allowed to cool to room temperature and poured into H₂O (100 mL). The resulting solution was extracted with EtOAc (3×), then the combined organic phases were washed with water and brine, dried over anhyd. Na₂CO₃, and concentrated. The crude residue was purified by silica gel chromatography (100% hexanes to 100% EtOAc) to afford the desired product as a light brown solid (3.39 g; 66%).

Step g: To a mixture of 2-bromo-5,6,8,9-tetrahydro-7H-benzocyclohepten-7-one (1.03 g, 4.31 mmol) and pyrrolidine (0.43 mL, 5.17 mmol) in DCE (21.5 mL) was added AcOH (0.25 mL, 4.31 mmol) followed by NaBH(OAc)₃ (1.19 g, 5.60 mmol). The reaction was stirred at r.t. for 16 h and carefully quenched with H₂O followed by sat. aq. NaHCO₃. The layers were separated and the aqueous layer was extracted with CH₂Cl₂ (2×20 mL). The combined organic layers were washed with brine, dried over anhyd. Na₂SO₄, and concentrated. The residue was purified by silica gel chromatography (100% CH₂Cl₂ to 10% MeOH in CH₂Cl₂, 0.5% NEt₃) to afford the desired product as a viscous orange oil (978 mg; 77%).

Step h: To a mixture of the product of step g (191 mg, 0.649 mmol), B₂pin₂ (214 mg, 0.844 mmol), and KOAc (83 mg, 0.844 mmol) was added dioxane (6.5 mL), then the suspension was degassed with N₂ for 10 min. (dppf)PdCl₂ (24 mg, 0.0325 mmol) was added and the reaction mixture was stirred at 90° C. for 3 h. Upon cooling, EtOAc (20 mL) was added and the mixture was filtered through celite. The filtrate was concentrated to afford the crude material as a viscous brown oil.

Step i: To a mixture of the product of step f (144 mg, 0.295 mmol), the crude product of step h (0.325 mmol), and Na₂CO₃ (63 mg, 0.590 mmol) was added dioxane (5.3 mL) and H₂O (0.60 mL), then the suspension was degassed with N₂ for 10 min. (dppf)PdCl₂ (11 mg, 0.0148 mmol) was added and the reaction mixture was stirred at 80° C. for 14 h. Upon cooling, CH₂Cl₂ (15 mL) was added and the mixture was dried over anhyd. MgSO₄, filtered, and concentrated. The residue was purified by silica gel chromatography (100% CH₂Cl₂ to 10% MeOH in CH₂Cl₂, 1% NH₃) to afford the desired product as a brown solid (127 mg; 69%).

Step j: To a solution of the product of step i (129 mg, 0.207 mmol) in CH₂Cl₂ (1.1 mL) was added TFA (1.1 mL). The reaction was stirred for 2 h at r.t. then concentrated. To the residue was added NH₃ in MeOH (7 N solution, 2.1 mL) and the reaction mixture stirred at r.t. for 14 h. Upon cooling, the reaction mixture was concentrated. Purification by C18 reverse phase chromatography (100% H₂O to 100% ACN, 0.1% TFA) and reverse phase HPLC (10 to 70% ACN in H₂O, 0.1% TFA) then lyophilization provided the title compound as a light yellow solid (5 mg, 4%). ¹H NMR (400 MHz, Methanol-d₄) δ 8.79 (d, J=2.0 Hz, 1H), 8.58 (d, J=2.0 Hz, 1H), 8.00 (d, J=8.2 Hz, 1H), 7.83 (dd, J=8.2, 1.8 Hz, 1H), 7.69 (d, J=1.7 Hz, 1H), 7.54 (d, J=2.0 Hz, 1H), 7.51 (dd, J=7.7, 2.0 Hz, 1H), 7.31 (d, J=7.7 Hz, 1H), 4.46 (dd, J=5.3, 4.3 Hz, 2H), 3.65-3.53 (m, 3H), 3.51 (dd, J=5.2, 4.4 Hz, 2H), 3.29-3.17 (m, 2H), 3.11-2.86 (m, 4H), 2.53-2.42 (m, 2H), 2.22-2.05 (m, 2H), 2.03-1.95 (m, 2H), 1.57 (p, J=11.6, 11.2 Hz, 2H). ESI MS [M+H]⁺ for C₃₀H₃₂N₅O₂, calcd. 494.3, found 494.2.

### Example 2: 8-{5-[6-(Pyrrolidin-1-yl)-5,6,7,8-tetrahydronaphthalen-2-yl]-1H-pyrazolo[3,4-b]pyridin-3-yl}-2,3,4,5-tetrahydro-1,4-benzoxazepin-5-one

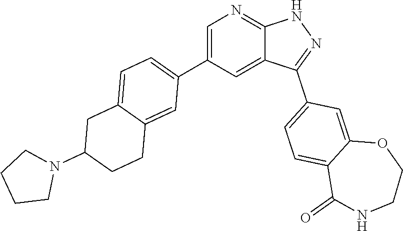 [SMILES: O=C1NCCOc2cc(-c3n[nH]c4ncc(-c5ccc6c(c5)CCC(N5CCCC5)C6)cc34)ccc21]

The title compound was prepared in a similar manner to Example 1. ¹H NMR (400 MHz, Methanol-d₄) δ 8.75 (d, J=2.0 Hz, 1H), 8.54 (d, J=2.1 Hz, 1H), 7.99 (dd, J=8.2, 0.4 Hz, 1H), 7.81 (dd, J=8.2, 1.7 Hz, 1H), 7.67 (d, J=1.3 Hz, 1H), 7.53-7.46 (m, 2H), 7.27 (d, J=7.9 Hz, 1H), 4.46 (dd, J=5.5, 4.0 Hz, 2H), 3.86-3.73 (m, 2H), 3.66-3.55 (m, 1H), 3.51 (dd, J=5.6, 4.1 Hz, 2H), 3.41-3.34 (m, 1H), 3.30-3.23 (m, 2H), 3.17-2.94 (m, 3H), 2.51-2.39 (m, 1H), 2.30-2.16 (m, 2H), 2.14-2.01 (m, 2H), 1.94 (ddt, J=17.2, 11.8, 5.7 Hz, 1H). ESI MS [M+H]⁺ for C₂₉H₃₀N₅O₂, calcd. 480.2, found 480.2.

### Example 3: 8-{5-[7-(Pyrrolidin-1-yl)-5,6,7,8-tetrahydronaphthalen-2-yl]-1H-pyrazolo[3,4-b]pyridin-3-yl}-2,3,4,5-tetrahydro-1,4-benzoxazepin-5-one

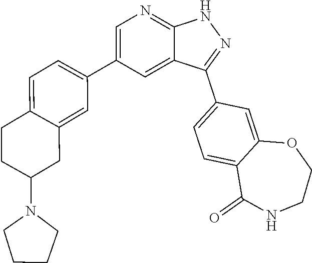 [SMILES: O=C1NCCOc2cc(-c3n[nH]c4ncc(-c5ccc6c(c5)CC(N5CCCC5)CC6)cc34)ccc21]

The title compound was prepared in a similar manner to Example 1. ¹H NMR (400 MHz, DMSO-d₆) δ 9.73 (s, 1H), 8.87 (d, J=2.1 Hz, 1H), 8.67 (d, J=2.1 Hz, 1H), 8.41 (t, J=5.4 Hz, 1H), 7.96 (d, J=8.2 Hz, 1H), 7.89 (dd, J=8.2, 1.7 Hz, 1H), 7.69-7.65 (m, 2H), 7.63 (s, 1H), 7.30 (d, J=8.0 Hz, 1H), 4.38 (dd, J=5.3, 4.1 Hz, 2H), 3.72-3.58 (m, 3H), 3.47-3.30 (m, 3H), 3.30-3.12 (m, 2H), 3.08-2.96 (m, 2H), 2.95-2.83 (m, 1H), 2.40-2.29 (m, 1H), 2.14-2.00 (m, 2H), 1.98-1.76 (m, 3H). ESI MS [M+H]⁺ for C₂₉H₃₀N₅O₂, calcd. 480.2, found 480.2.

### Example 4: 8-[5-(6-{[(3S)—Oxolan-3-yl]amino}-5,6,7,8-tetrahydronaphthalen-2-yl)-1H-pyrazolo[3,4-b]pyridin-3-yl]-2,3,4,5-tetrahydro-1,4-benzoxazepin-5-one

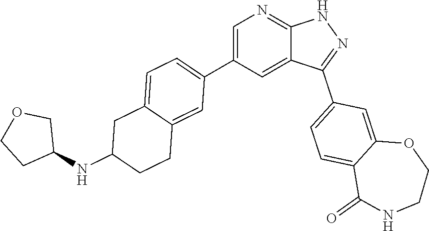 [SMILES: O=C1NCCOc2cc(-c3n[nH]c4ncc(-c5ccc6c(c5)CCC(N[C@H]5CCOC5)C6)cc34)ccc21]

The title compound was prepared in a similar manner to Example 1. ¹H NMR (400 MHz, Methanol-d₄) δ 9.17 (d, J=1.9 Hz, 1H), 9.11 (d, J=1.9 Hz, 1H), 8.06 (dd, J=8.2, 0.4 Hz, 1H), 7.85 (dd, J=8.2, 1.7 Hz, 1H), 7.72 (dd, J=1.7, 0.4 Hz, 1H), 7.67-7.58 (m, 2H), 7.37 (d, J=7.6 Hz, 1H), 4.51-4.46 (m, 2H), 4.28-4.20 (m, 1H), 4.14-4.01 (m, 2H), 3.93 (dt, J=10.9, 5.7 Hz, 1H), 3.79 (ddd, J=8.9, 8.2, 7.3 Hz, 1H), 3.75-3.65 (m, 1H), 3.53 (dd, J=5.5, 4.1 Hz, 2H), 3.43 (dd, J=16.2, 5.4 Hz, 1H), 3.22-2.95 (m, 3H), 2.54-2.38 (m, 2H), 2.21-2.05 (m, 1H), 1.94 (qd, J=11.6, 5.9 Hz, 1H). ESI MS [M+H]⁺ for C₂₉H₃₀N₅O₃, calcd. 496.2, found 496.2.

### Example 5: 8-[5-(6-{[(3R)—Oxolan-3-yl]amino}-5,6,7,8-tetrahydronaphthalen-2-yl)-1H-pyrazolo[3,4-b]pyridin-3-yl]-2,3,4,5-tetrahydro-1,4-benzoxazepin-5-one

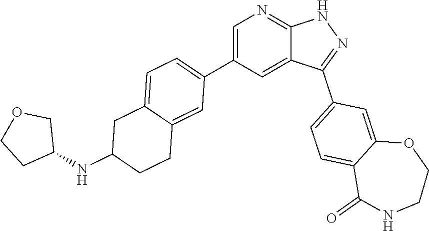 [SMILES: O=C1NCCOc2cc(-c3n[nH]c4ncc(-c5ccc6c(c5)CCC(N[C@@H]5CCOC5)C6)cc34)ccc21]

The title compound was prepared in a similar manner to Example 1. ¹H NMR (400 MHz, DMSO-d₆) δ 9.31-8.95 (m, 2H), 8.87 (d, J=2.1 Hz, 1H), 8.67 (d, J=2.1 Hz, 1H), 8.41 (t, J=5.3 Hz, 1H), 7.96 (d, J=8.2 Hz, 1H), 7.89 (dd, J=8.2, 1.7 Hz, 1H), 7.68 (dd, J=1.7, 0.3 Hz, 1H), 7.67-7.62 (m, 2H), 7.29 (d, J=7.8 Hz, 1H), 4.38 (dd, J=5.4, 4.1 Hz, 2H), 4.17-4.07 (m, 1H), 4.00-3.83 (m, 3H), 3.70 (q, J=7.7 Hz, 1H), 3.65-3.47 (m, 2H), 3.37-3.26 (m, 2H), 3.09-2.86 (m, 3H), 2.38-2.24 (m, 2H), 2.14-2.01 (m, 1H), 1.90-1.76 (m, 1H). ESI MS [M+H]⁺ for C₂₉H₃₀N₅O₃, calcd. 496.2, found 496.2.

### Example 6: 6-Fluoro-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

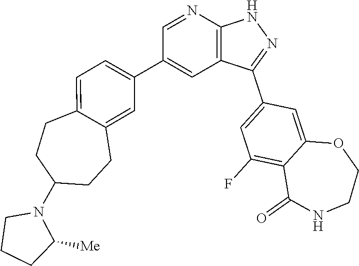 [SMILES: C[C@@H]1CCCN1C1CCc2ccc(-c3cnc4[nH]nc(-c5cc(F)c6c(c5)OCCNC6=O)c4c3)cc2CC1]

The title compound was prepared in a similar manner to Example 1. ¹H NMR (400 MHz, DMSO-d₆) δ 8.87 (d, J=2.0 Hz, 1H), 8.72 (d, J=2.1 Hz, 1H), 8.55 (t, J=6.1 Hz, 1H), 7.80 (dd, J=10.8, 1.5 Hz, 1H), 7.65-7.61 (m, 2H), 7.57 (dd, J=7.6, 2.0 Hz, 1H), 7.27 (dd, J=7.8, 1.3 Hz, 1H), 4.27 (t, J=5.5 Hz, 2H), 3.33-3.29 (m, 2H), 3.01-2.77 (m, 5H), 2.77-2.65 (m, 2H), 2.44 (q, J=8.3 Hz, 1H), 2.07-1.94 (m, 2H), 1.88-1.75 (m, 1H), 1.68-1.49 (m, 2H), 1.49-1.37 (m, 1H), 1.34-1.21 (m, 2H), 1.02 (d, J=6.0 Hz, 3H). ESI MS [M+H]⁺ for C₃₁H₃₃FN₅O₂, calcd. 526.3, found 526.3.

### Example 7: 8-[5-(3-Cyclopentyl-2,3,4,5-tetrahydro-1H-3-benzazepin-7-yl)-1H-pyrazolo[3,4-b]pyridin-3-yl]-2,3,4,5-tetrahydro-1,4-benzoxazepin-5-one

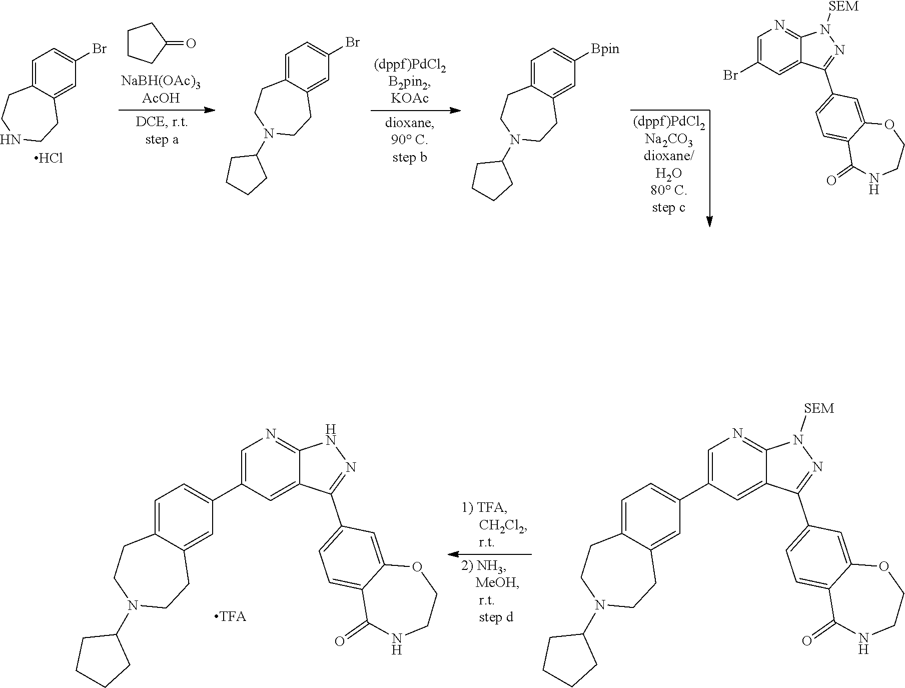

Step a: To a mixture of 7-bromo-2,3,4,5-tetrahydro-1H-3-benzazepine hydrochloride (272 mg, 1.04 mmol) and cyclopentanone (0.11 mL, 1.29 mmol) in DCE (5.2 mL) was added AcOH (60 μL, 1.04 mmol) followed by NaBH(OAc)₃ (331 mg, 1.56 mmol). The reaction was stirred at r.t. for 17 h, then carefully quenched with sat. aq. NaHCO₃. The layers were separated, and the aqueous layer was extracted with CH₂Cl₂ (2×10 mL). The combined organic layers were washed with brine, dried over anhyd. MgSO₄, and concentrated to afford the desired product as a colorless oil (293 mg, 96%).

Step b: To a mixture of the product of step a (111 mg, 0.377 mmol), B₂pin₂ (129 mg, 0.490 mmol), and KOAc (48 mg, 0.490 mmol) was added dioxane (3.8 mL), then the suspension was degassed with N₂ for 10 min. (dppf)PdCl₂ (14 mg, 0.0189 mmol) was added and the reaction mixture was stirred at 90° C. for 3 h. Upon cooling, EtOAc (15 mL) was added and the mixture was filtered through celite. The filtrate was concentrated to afford the crude material as a viscous brown oil.

Step c: To a mixture of the product of Example 1, step f (168 mg, 0.343 mmol), the crude product of step b (0.377 mmol), and Na₂CO₃ (73 mg, 0.685 mmol) was added dioxane (6.2 mL) and H₂O (0.70 mL), then the suspension was degassed with N₂ for 10 min. (dppf)PdCl₂ (13 mg, 0.0148 mmol) was added and the reaction mixture was stirred at 80° C. for 14 h. Upon cooling, CH₂Cl₂ (15 mL) was added and the mixture was dried over anhyd. MgSO₄, filtered, and concentrated. The residue was purified by silica gel chromatography (100% CH₂Cl₂ to 10% MeOH in CH₂Cl₂, 1% NH₃) to afford the desired product as a brown solid (144 mg; 67%).

Step d: To a solution of the product of step c (144 mg, 0.231 mmol) in CH₂Cl₂ (1.1 mL) was added TFA (1.1 mL). The reaction was stirred for 1.5 h at r.t. then concentrated. To the residue was added NH₃ in MeOH (7 N solution, 2.3 mL) and the reaction mixture stirred at r.t. for 14 h. Upon cooling, the reaction was concentrated. Purification by C18 reverse phase chromatography (100% H₂O to 100% ACN, 0.1% TFA) and reverse phase HPLC (10 to 90% ACN in H₂O, 0.1% TFA) then lyophilization provided the title compound as a light yellow solid (44 mg, 31%). ¹H NMR (400 MHz, Methanol-d₄) δ 8.81 (d, J=2.1 Hz, 1H), 8.61 (d, J=2.1 Hz, 1H), 8.01 (dd, J=8.2, 0.4 Hz, 1H), 7.85 (dd, J=8.2, 1.7 Hz, 1H), 7.71 (dd, J=1.7, 0.4 Hz, 1H), 7.60-7.43 (m, 2H), 7.28 (d, J=7.6 Hz, 1H), 4.46 (dd, J=5.5, 4.0 Hz, 2H), 3.51 (dd, J=5.4, 4.2 Hz, 2H), 3.12-3.02 (m, 5H), 3.00-2.71 (m, 4H), 2.06-1.93 (m, 2H), 1.84-1.69 (m, 2H), 1.69-1.46 (m, 4H). ESI MS [M+H]⁺ for C₃₀H₃₂N₅O₂, calcd. 494.3, found 494.2.

### Example 8: 8-[5-(8-Chloro-2-cyclopentyl-1,2,3,4-tetrahydroisoquinolin-6-yl)-1H-pyrazolo[3,4-b]pyridin-3-yl]-2,3,4,5-tetrahydro-1,4-benzoxazepin-5-one

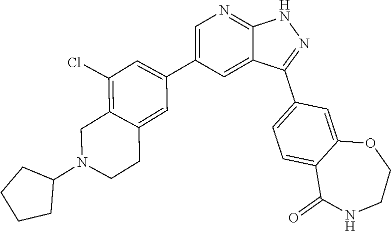 [SMILES: O=C1NCCOc2cc(-c3n[nH]c4ncc(-c5cc(Cl)c6c(c5)CCN(C5CCCC5)C6)cc34)ccc21]

The title compound was prepared in a similar manner to Example 7. ¹H NMR (400 MHz, DMSO-d₆) δ 10.02 (brs, 1H), 8.96 (d, J=2.1 Hz, 1H), 8.82 (d, J=2.1 Hz, 1H), 8.42 (t, J=5.3 Hz, 1H), 8.05 (d, J=1.7 Hz, 1H), 7.98 (d, J=8.2 Hz, 1H), 7.92 (dd, J=8.2, 1.7 Hz, 1H), 7.86 (d, J=1.4 Hz, 1H), 7.71 (d, J=1.6 Hz, 1H), 4.57 (d, J=16.9 Hz, 1H), 4.45-4.35 (m, 3H), 3.87-3.76 (m, 2H), 3.43-3.34 (m, 3H), 3.30-3.21 (m, 2H), 2.23-2.09 (m, 2H), 1.96-1.69 (m, 4H), 1.69-1.52 (m, 2H). ESI MS [M+H]⁺ for C₂₉H₂₉ClN₅O₂, calcd. 514.2, found 514.2.

### Example 9: 8-(5-(2-Cyclopentyl-1,2,3,4-tetrahydroisoquinolin-6-yl)-1H-pyrazolo[3,4-b]pyridin-3-yl)-3,4-dihydrobenzo[f][1,4]oxazepin-5(2H)-one

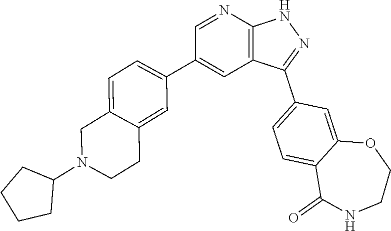 [SMILES: O=C1NCCOc2cc(-c3n[nH]c4ncc(-c5ccc6c(c5)CCN(C5CCCC5)C6)cc34)ccc21]

The title compound was prepared in a similar manner to Example 7. ¹H NMR (400 MHz, DMSO-d₆) δ 8.82 (d, J=2.1 Hz, 1H), 8.63 (d, J=2.1 Hz, 1H), 8.36 (t, J=5.4 Hz, 1H), 7.95-7.82 (m, 2H), 7.65 (d, J=1.6 Hz, 1H), 7.55 (d, J=8.0 Hz, 2H), 7.17 (d, J=7.8 Hz, 1H), 4.34 (dd, J=5.3, 4.1 Hz, 2H), 3.63 (s, 2H), 3.40-3.33 (m, 2H), 2.87 (t, J=5.9 Hz, 2H), 2.73-2.58 (m, 3H), 1.87 (d, J=6.3 Hz, 2H), 1.62 (d, J=7.3 Hz, 2H), 1.57-1.34 (m, 4H). ESI MS [M+H]⁺ for C₂₉H₃₀N₅O₂, calcd. 480.2, found 480.2.

### Example 10: 8-(5-(2-Cyclopentyl-1,2,3,4-tetrahydroisoquinolin-7-yl)-1H-pyrazolo[3,4-b]pyridin-3-yl)-3,4-dihydrobenzo[f][1,4]oxazepin-5(2H)-one

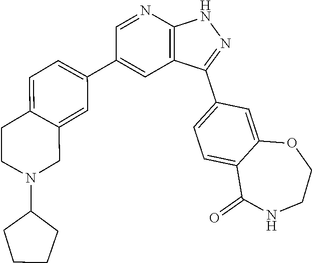 [SMILES: O=C1NCCOc2cc(-c3n[nH]c4ncc(-c5ccc6c(c5)CN(C5CCCC5)CC6)cc34)ccc21]

The title compound was prepared in a similar manner to Example 7. ¹H NMR (400 MHz, DMSO-d₆) δ 8.81 (d, J=2.1 Hz, 1H), 8.62 (d, J=2.1 Hz, 1H), 8.35 (t, J=5.4 Hz, 1H), 7.95-7.82 (m, 2H), 7.64 (dd, J=1.7, 0.5 Hz, 1H), 7.59-7.48 (m, 2H), 7.20 (d, J=7.9 Hz, 1H), 4.34 (dd, J=5.4, 4.1 Hz, 2H), 3.68 (s, 2H), 3.35 (q, J=5.0 Hz, 2H), 2.85-2.78 (m, 2H), 2.73-2.58 (m, 3H), 1.88 (d, J=5.7 Hz, 2H), 1.62 (d, J=7.3 Hz, 2H), 1.58-1.35 (m, 4H). ESI MS [M+H]⁺ for C₂₉H₃₀N₅O₂, calcd. 480.2, found 480.2.

### Example 11: 8-(5-(3-(Oxetan-3-yl)-2,3,4,5-tetrahydro-1H-benzo[d]azepin-7-yl)-1H-pyrazolo[3,4-b]pyridin-3-yl)-3,4-dihydrobenzo[f][1,4]oxazepin-5(2H)-one

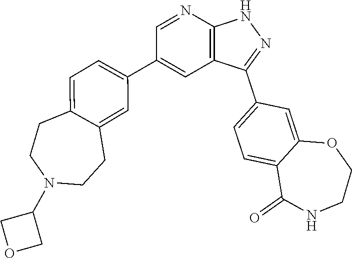 [SMILES: O=C1NCCOc2cc(-c3n[nH]c4ncc(-c5ccc6c(c5)CCN(C5COC5)CC6)cc34)ccc21]

The title compound was prepared in a similar manner to Example 7. ¹H NMR (400 MHz, DMSO-d₆) δ 8.86 (d, J=2.1 Hz, 1H), 8.66 (d, J=2.1 Hz, 1H), 8.38 (t, J=5.4 Hz, 1H), 7.96-7.89 (m, 1H), 7.85 (dd, J=8.2, 1.7 Hz, 1H), 7.75 (d, J=2.0 Hz, 1H), 7.72-7.61 (m, 2H), 7.37 (d, J=7.9 Hz, 1H), 4.83 (t, J=7.1 Hz, 2H), 4.72 (t, J=7.5 Hz, 2H), 4.44 (s, 1H), 4.38-4.31 (m, 2H), 3.36 (q, J=5.0 Hz, 2H), 3.29-2.94 (m, 6H), 2.91 (s, 2H). ESI MS [M+H]⁺ for C₂₈H₂₈N₅O₃, calcd. 482.2, found 482.2.

### Example 12: 8-(5-(2-(Oxetan-3-yl)-1,2,3,4-tetrahydroisoquinolin-6-yl)-1H-pyrazolo[3,4-b]pyridin-3-yl)-3,4-dihydrobenzo[f][1,4]oxazepin-5(2H)-one

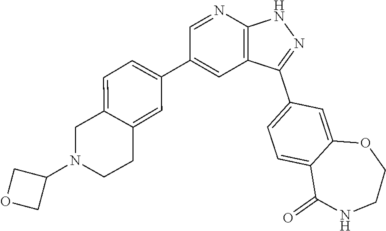 [SMILES: O=C1NCCOc2cc(-c3n[nH]c4ncc(-c5ccc6c(c5)CCN(C5COC5)C6)cc34)ccc21]

The title compound was prepared in a similar manner to Example 7. ¹H NMR (400 MHz, DMSO-d₆) δ 11.13 (s, 1H), 8.88 (d, J=2.1 Hz, 1H), 8.70 (d, J=2.1 Hz, 1H), 8.38 (t, J=5.4 Hz, 1H), 7.93 (dd, J=8.2, 0.4 Hz, 1H), 7.87 (dd, J=8.2, 1.7 Hz, 1H), 7.76 (d, J=7.7 Hz, 2H), 7.65 (dd, J=1.7, 0.5 Hz, 1H), 7.30 (d, J=8.1 Hz, 1H), 4.81 (d, J=6.5 Hz, 4H), 4.56 (d, J=23.2 Hz, 2H), 4.38-4.31 (m, 2H), 4.24 (s, 1H), 3.36 (q, J=5.1 Hz, 2H), 3.17 (s, 4H). ESI MS [M+H]⁺ for C₂₇H₂₆N₅O₃, calcd. 468.2, found 468.2.

### Example 13: 8-(5-(2-(2-Methoxyethyl)-1,2,3,4-tetrahydroisoquinolin-6-yl)-1H-pyrazolo[3,4-b]pyridin-3-yl)-3,4-dihydrobenzo[f][1,4]oxazepin-5(2H)-one

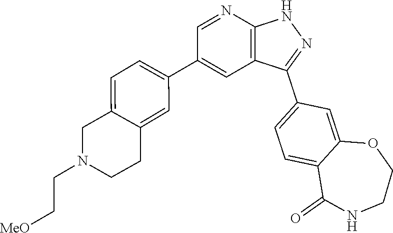 [SMILES: COCCN1CCc2cc(-c3cnc4[nH]nc(-c5ccc6c(c5)OCCNC6=O)c4c3)ccc2C1]

The title compound was prepared in a similar manner to Example 7. ¹H NMR (400 MHz, DMSO-d₆) δ 10.07 (s, 1H), 8.88 (d, J=2.1 Hz, 1H), 8.70 (d, J=2.1 Hz, 1H), 8.38 (t, J=5.4 Hz, 1H), 7.93 (dd, J=8.2, 0.4 Hz, 1H), 7.87 (dd, J=8.2, 1.7 Hz, 1H), 7.79-7.72 (m, 2H), 7.65 (dd, J=1.7, 0.5 Hz, 1H), 7.34 (d, J=8.7 Hz, 1H), 4.58 (d, J=15.5 Hz, 1H), 4.44-4.31 (m, 3H), 3.84-3.69 (m, 3H), 3.53-3.33 (m, 7H), 3.26-3.07 (m, 3H). ESI MS [M+H]⁺ for C₂₇H₂₈N₅O₃, calcd. 470.2, found 470.2.

### Example 14: 3-Cyclopentyl-7-(3-(2,2-difluorobenzo[d][1,3]dioxol-5-yl)-1H-pyrazolo[3,4-b]pyridin-5-yl)-2,3,4,5-tetrahydro-1H-benzo[d]azepine

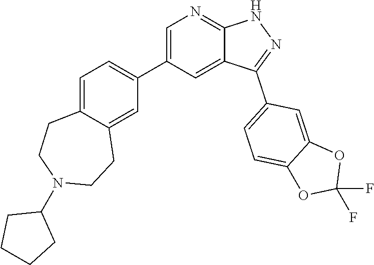 [SMILES: FC1(F)Oc2ccc(-c3n[nH]c4ncc(-c5ccc6c(c5)CCN(C5CCCC5)CC6)cc34)cc2O1]

The title compound was prepared in a similar manner to Example 7. ¹H NMR (400 MHz, DMSO-d₆) δ 13.98 (s, 1H), 9.67 (s, 1H), 8.86 (d, J=2.0 Hz, 1H), 8.69 (d, J=2.1 Hz, 1H), 8.07 (dd, J=1.7, 0.4 Hz, 1H), 7.93 (dd, J=8.4, 1.7 Hz, 1H), 7.77-7.66 (m, 2H), 7.55 (dd, J=8.4, 0.4 Hz, 1H), 7.35 (d, J=7.8 Hz, 1H), 3.67 (s, 2H), 3.15 (qd, J=28.3, 27.2, 14.3 Hz, 7H), 2.02 (d, J=9.7 Hz, 2H), 1.72 (d, J=14.7 Hz, 4H), 1.54 (s, 2H). ESI MS [M+H]⁺ for C₂₇H₂₇F₂N₄O₂, calcd. 489.2, found 489.2.

### Example 15: 8-(5-(3-(2-Methoxyethyl)-2,3,4,5-tetrahydro-1H-benzo[d]azepin-7-yl)-1H-pyrazolo[3,4-b]pyridin-3-yl)-3,4-dihydrobenzo[f][1,4]oxazepin-5(2H)-one

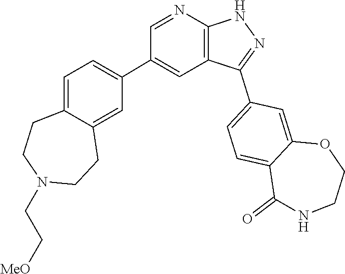

The title compound was prepared in a similar manner to Example 7. ¹H NMR (400 MHz, DMSO-d₆) δ 9.87 (s, 1H), 8.85 (d, J=2.1 Hz, 1H), 8.65 (d, J=2.1 Hz, 1H), 8.38 (t, J=5.4 Hz, 1H), 7.93 (dd, J=8.1, 0.4 Hz, 1H), 7.85 (dd, J=8.2, 1.7 Hz, 1H), 7.74-7.61 (m, 3H), 7.34 (d, J=7.8 Hz, 1H), 4.38-4.31 (m, 2H), 3.73-3.65 (m, 3H), 3.31 (s, 9H), 3.11 (td, J=17.0, 16.1, 8.0 Hz, 4H). ESI MS [M+H]⁺ for C₂₈H₃₀N₅O₃, calcd. 484.2, found 484.2.

### Example 16: 8-{5-[3-(2-Methylpropanoyl)-2,3,4,5-tetrahydro-1H-3-benzazepin-7-yl]-1H-pyrazolo[3,4-b]pyridin-3-yl}-2,3,4,5-tetrahydro-1,4-benzoxazepin-5-one

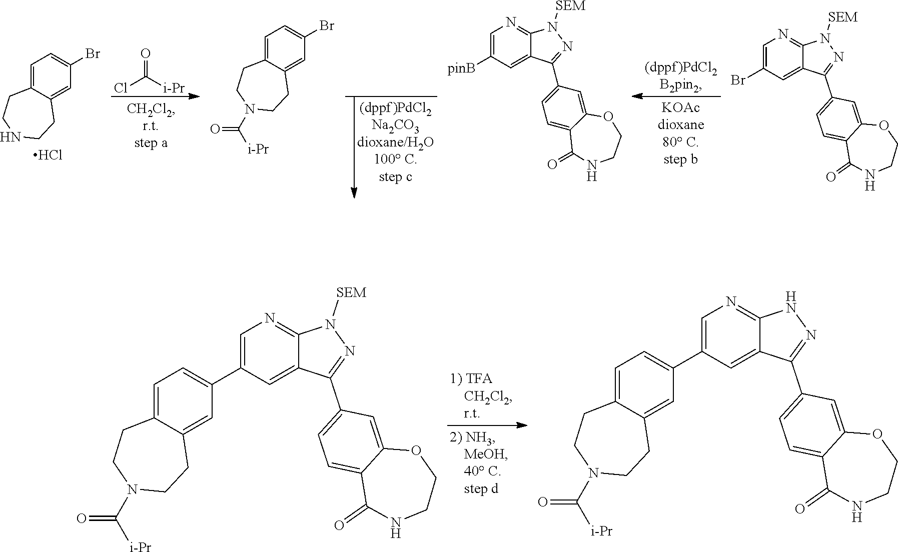

Step a: To a suspension of 7-bromo-2,3,4,5-tetrahydro-1H-3-benzazepine hydrochloride (84 mg, 0.320 mmol) in CH₂Cl₂ (3.2 mL) was added NEt₃ (0.13 mL, 0.960 mmol) followed by 2-methylpropanoyl chloride (40 μL, 0.384 mmol). The reaction was stirred at r.t. for 17 h, then carefully quenched with sat. aq. NH₄Cl. The layers were separated and the aqueous layer was extracted with CH₂Cl₂ (2×5 mL). The combined organic layers were washed with brine, dried over anhyd. MgSO₄, and concentrated. The crude residue was dissolved in CH₂Cl₂ (5 mL) and washed with sat. aq. NaHCO₃, then the aqueous layer was extracted with CH₂Cl₂ (2×5 mL). The combined organic layers were washed with brine, dried over anhyd. MgSO₄, and concentrated to afford the desired product as a slightly pink, viscous oil (94 mg, 99%).

Step b: To a mixture of the product of Example 1, step f (481 mg, 0.983 mmol), B₂pin₂ (300 mg, 1.18 mmol), and KOAc (125 mg, 1.28 mmol) was added dioxane (9.8 mL), then the suspension was degassed with N₂ for 10 min. (dppf)PdCl₂ (36 mg, 0.0492 mmol) was added and the reaction mixture was stirred at 80° C. for 4 h. Upon cooling, EtOAc (30 mL) was added and the mixture was filtered through celite. The filtrate was concentrated to afford the crude material as a viscous brown oil.

Step c: To a mixture of the product of step a (94 mg, 0.320 mmol), the crude product of step b (0.246 mmol), and Na₂CO₃ (74 mg, 0.492 mmol) was added dioxane (4.4 mL) and H₂O (0.50 mL), then the suspension was degassed with N₂ for 10 min. (dppf)PdCl₂ (9 mg, 0.0123 mmol) was added and the reaction mixture was stirred at 100° C. for 4 h. Upon cooling, CH₂Cl₂ (15 mL) was added and the mixture was dried over anhyd. MgSO₄, filtered, and concentrated. The residue was purified by silica gel chromatography (100% hexanes to 100% EtOAc to 10% MeOH in EtOAc) to afford the desired product as a light brown solid (105 mg; 68%).

Step d: To a solution of the product of step c (105 mg, 0.168 mmol) in CH₂Cl₂ (1.7 mL) was added TFA (1.7 mL). The reaction was stirred for 2 h at r.t. then concentrated. To the residue was added NH₃ in MeOH (7 N solution, 3.4 mL) and the reaction mixture stirred at 40° C. for 2 h. Upon cooling, the reaction was concentrated and purified by silica gel chromatography (100% CH₂Cl₂ to 10% MeOH in CH₂Cl₂) and dried in vacuo to provide the title compound as an off-white solid (29 mg, 35%). ¹H NMR (400 MHz, DMSO-d₆) δ 8.88 (d, J=2.0 Hz, 1H), 8.69 (d, J=2.1 Hz, 1H), 8.40 (t, J=5.4 Hz, 1H), 7.95 (d, J=8.2 Hz, 1H), 7.90 (dd, J=8.2, 1.7 Hz, 1H), 7.69-7.65 (m, 2H), 7.62 (dd, J=7.8, 1.9 Hz, 1H), 7.31 (dd, J=7.7, 3.6 Hz, 1H), 4.38 (dd, J=5.3, 4.1 Hz, 2H), 3.65 (dt, J=17.6, 8.3 Hz, 4H), 3.43-3.34 (m, 2H), 3.10-2.83 (m, 5H), 1.03 (dd, J=6.7, 2.9 Hz, 6H). ESI MS [M+H]⁺ for C₂₉H₃₀N₅O₃, calcd. 496.2, found 496.2.

### Example 17: 8-[5-(3-Cyclopropanecarbonyl-2,3,4,5-tetrahydro-1H-3-benzazepin-7-yl)-1H-pyrazolo[3,4-b]pyridin-3-yl]-2,3,4,5-tetrahydro-1,4-benzoxazepin-5-one

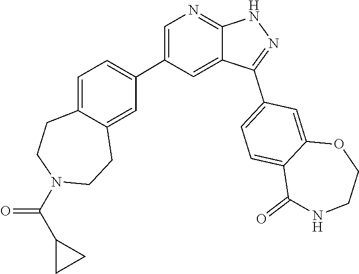

The title compound was prepared in a similar manner to Example 16. ¹H NMR (400 MHz, DMSO-d₆) δ 8.88 (s, 1H), 8.69 (s, 1H), 8.40 (t, J=5.3 Hz, 1H), 7.95 (d, J=8.1 Hz, 1H), 7.90 (dd, J=8.2, 1.7 Hz, 1H), 7.74-7.65 (m, 2H), 7.62 (dd, J=7.7, 2.0 Hz, 1H), 7.32 (t, J=8.5 Hz, 1H), 4.38 (dd, J=5.4, 4.0 Hz, 2H), 3.85 (t, J=8.0 Hz, 2H), 3.64 (t, J=8.5 Hz, 2H), 3.43-3.38 (m, 2H), 3.12-2.98 (m, 2H), 2.98-2.85 (m, 2H), 2.21-1.92 (m, 1H), 0.89-0.55 (m, 4H). ESI MS [M+H]⁺ for C₂₉H₂₈N₅O₃, calcd. 494.2, found 494.2.

### Example 18: 8-[5-(3-Methanesulfonyl-2,3,4,5-tetrahydro-1H-3-benzazepin-7-yl)-1H-pyrazolo[3,4-b]pyridin-3-yl]-2,3,4,5-tetrahydro-1,4-benzoxazepin-5-one

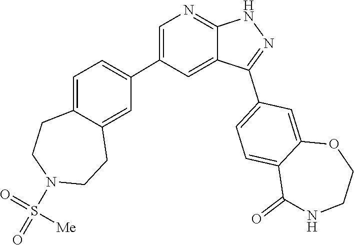

The title compound was prepared in a similar manner to Example 16. ¹H NMR (400 MHz, DMSO-d₆) δ 8.88 (d, J=2.1 Hz, 1H), 8.69 (d, J=2.1 Hz, 1H), 8.40 (t, J=5.3 Hz, 1H), 7.96 (d, J=8.2 Hz, 1H), 7.90 (dd, J=8.2, 1.7 Hz, 1H), 7.70 (d, J=2.0 Hz, 1H), 7.68 (d, J=1.7 Hz, 1H), 7.65 (dd, J=7.7, 2.0 Hz, 1H), 7.33 (d, J=7.8 Hz, 1H), 4.38 (dd, J=5.4, 4.1 Hz, 2H), 3.43-3.35 (m, 6H), 3.17-2.98 (m, 4H), 2.89 (s, 3H). ESI MS [M+H]⁺ for C₂₆H₂₆N₅O₄S, calcd. 504.2, found 504.2.

### Example 19: 8-(5-(3-(2-Hydroxy-2-methylpropanoyl)-2,3,4,5-tetrahydro-1H-benzo[d]azepin-7-yl)-1H-pyrazolo[3,4-b]pyridin-3-yl)-3,4-dihydrobenzo[f][1,4]oxazepin-5(2H)-one

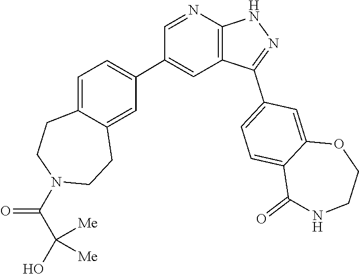

The title compound was prepared in a similar manner to Example 16. ¹H NMR (400 MHz, DMSO-d₆) δ 8.89-8.82 (m, 1H), 8.70-8.62 (m, 1H), 8.37 (t, J=5.3 Hz, 1H), 7.96-7.82 (m, 2H), 7.70-7.60 (m, 2H), 7.57 (dd, J=7.7, 2.0 Hz, 1H), 7.28 (d, J=7.9 Hz, 1H), 4.34 (dd, J=5.4, 4.1 Hz, 2H), 3.57 (s, 2H), 3.36 (q, J=5.1 Hz, 2H), 2.95 (s, 2H), 1.67-1.55 (m, 1H), 1.33 (s, 6H). ESI MS [M+H]⁺ for C₂₉H₃₀N₅O₄, calcd. 512.2, found 512.2.

### Example 20: 8-(5-(3-Cyclopropyl-2,3,4,5-tetrahydro-1H-benzo[d]azepin-7-yl)-1H-pyrazolo[3,4-b]pyridin-3-yl)-3,4-dihydrobenzo[f][1,4]oxazepin-5(2H)-one

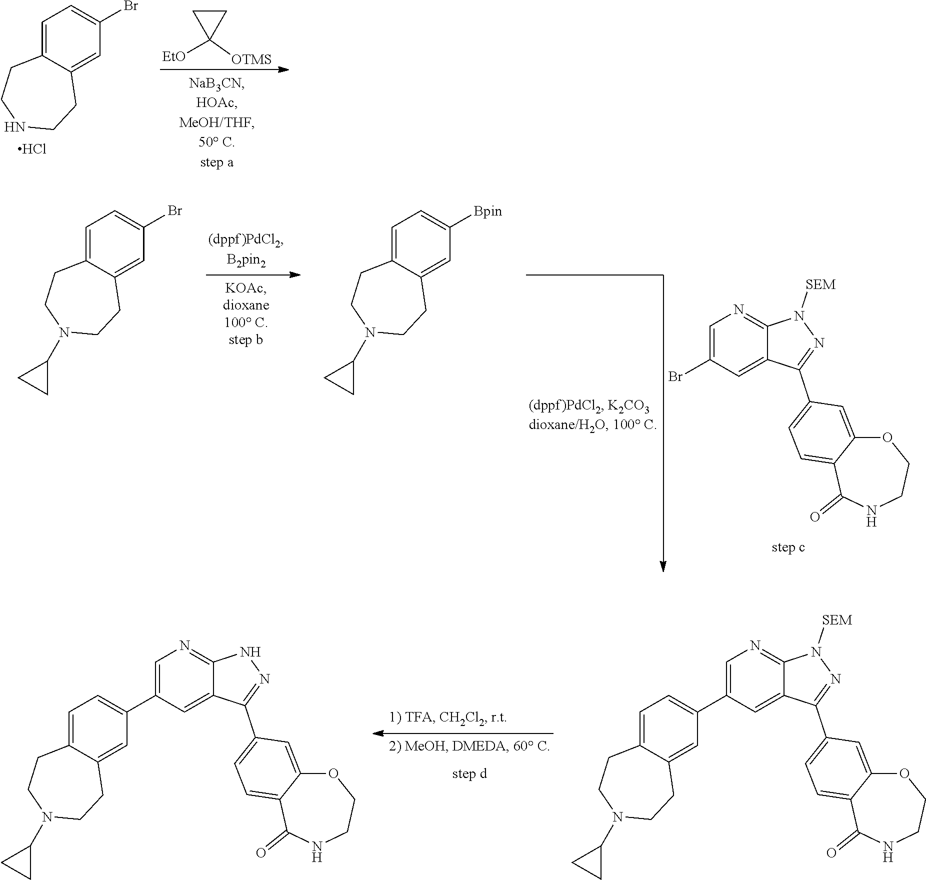

Step a: To a mixture of 7-bromo-2,3,4,5-tetrahydro-1H-3-benzazepine hydrochloride (100 mg, 0.38 mmol), (1-ethoxycyclopropoxy) trimethylsilane (264.9 mg, 1.5 mmol), and THF/MeOH (1:1, 1.5 mmol) was added AcOH (217.5 mL, 3.8 mmol) and NaBH₃CN (107.4 mg, 1.7 mmol) and heated at 50° C. for 24 h. After cooling to rt, the reaction mixture was filtered to remove any insoluble material, concentrated and purified by column chromatography (SiO₂, 0 to 100% CH₂Cl₂/MeOH/7N methanolic NH₃ (90:10:1) in CH₂Cl₂· to afford the desired product as a light brown oil (62 mg, 61%).

Step b: A mixture of the product from step a (62 mg, 0.23 mmol), B₂pin₂ (60 mg, 0.23 mmol), KOAc (46 mg, 0.47 mmol), and (dppf)PdCl₂ (9 mg, 0.01 mmol) was placed under nitrogen atmosphere. To this mixture was added degassed dioxane (1.5 mL) and heated at 100° C. for 6 h. After cooling to rt, the reaction mixture was filtered to remove any insoluble material, concentrated and used directly in the next step.

Step c: A mixture of the crude material obtained from step b (0.23 mmol assumed), the product of Example 1, step f (114 mg, 0.23 mmol), K₂CO₃ (65 mg, 0.47 mmol), and (dppf)PdCl₂ (9 mg, 0.01 mmol) was placed under nitrogen atmosphere. To this mixture was added degassed dioxane (1.5 mL) and H₂O (0.5 mL) and heated at 100° C. for 14h. After cooling to rt, EtOAc (20 mL) was added. The phases were separated, and the aq. phase was extracted with EtOAc (2×20 mL). The combined organic phases were dried over Na₂SO₄, concentrated and purified by column chromatography (SiO₂, 0 to 100% CH₂Cl₂/MeOH/7N methanolic NH₃ (90:10:1) in CH₂Cl₂· to afford the desired product as a tan solid (63 mg, 45%).

Step d: To the solution of product from step c (63 mg, 0.11 mmol) in CH₂Cl₂ (1.0 mL) was added TFA (1.0 mL). The reaction mixture was stirred at rt for 4 h. Solvent was removed, and the crude material was resuspended in MeOH (1.0 mL). To this mixture was added DMEDA (0.5 mL) and stirred at 60° C. for 1h. After cooling to rt, solvent was removed, and the crude material was purified by reversed phase HPLC using H₂O+0.1% TFA and CH₃CN+0.1% TFA as the mobile phase to obtain desired product as a yellow solid (15 mg; 26%). ¹H NMR (400 MHz, DMSO-d₆) δ 9.29 (s, 1H), 8.86 (d, J=2.1 Hz, 1H), 8.66 (d, J=2.1 Hz, 1H), 8.38 (t, J=5.4 Hz, 1H), 7.93 (dd, J=8.2, 0.4 Hz, 1H), 7.90-7.82 (m, 1H), 7.81-7.62 (m, 3H), 7.37 (d, J=7.9 Hz, 1H), 4.38-4.30 (m, 2H), 3.77 (s, 2H), 3.36 (q, J=5.0 Hz, 2H), 3.16 (dtd, J=29.8, 16.7, 15.5, 8.0 Hz, 6H), 2.94 (s, 1H), 1.04 (s, 2H), 0.87 (d, J=7.1 Hz, 2H). ESI MS [M+H]⁺ for C₂₈NH₂₈N₅O₂, calcd. 466.2, found 466.2.

### Example 21: 8-(5-(2-Cyclopropyl-1,2,3,4-tetrahydroisoquinolin-6-yl)-1H-pyrazolo[3,4-b]pyridin-3-yl)-3,4-dihydrobenzo[f][1,4]oxazepin-5(2H)-one

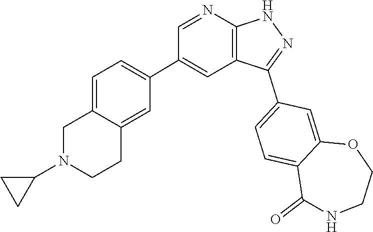

The title compound was prepared in a similar manner to Example 20. ¹H NMR (400 MHz, DMSO-d₆) δ 9.67 (s, 1H), 8.89 (d, J=2.1 Hz, 1H), 8.70 (d, J=2.1 Hz, 1H), 8.38 (t, J=5.4 Hz, 1H), 7.93 (dd, J=8.2, 0.4 Hz, 1H), 7.86 (dd, J=8.2, 1.7 Hz, 1H), 7.77 (d, J=5.0 Hz, 2H), 7.65 (dd, J=1.7, 0.5 Hz, 1H), 7.37 (d, J=8.6 Hz, 1H), 4.66 (d, J=16.3 Hz, 1H), 4.55 (s, 1H), 4.35 (dd, J=5.4, 4.1 Hz, 2H), 3.79 (s, 2H), 3.56 (s, 2H), 3.36 (q, J=5.0 Hz, 2H), 3.02 (s, 1H), 1.02 (d, J=11.8 Hz, 2H), 0.90 (d, J=7.3 Hz, 2H). ESI MS [M+H]⁺ for C₂₇H₂₆N₅O, calcd. 452.2, found 452.2.

### Example 22: 8-{5-[7-Methyl-7-(pyrrolidin-1-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

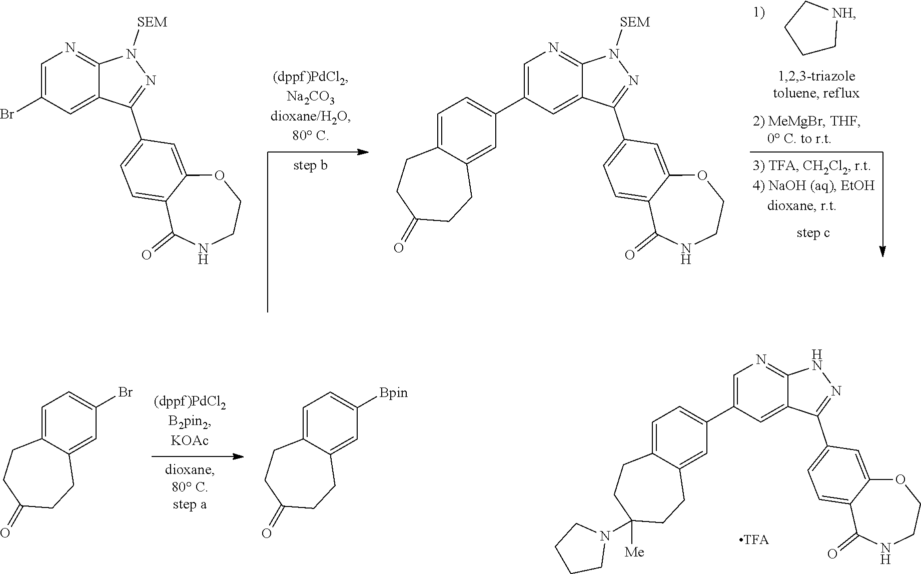

Step a: To a mixture of 2-bromo-5,6,8,9-tetrahydro-7H-benzocyclohepten-7-one (205 mg, 0.857 mmol), B₂pin₂ (218 mg, 0.857 mmol), and KOAc (93 mg, 0.943 mmol) was added dioxane (4.3 mL), then the suspension was degassed with N₂ for 10 min. (dppf)PdCl₂ (31 mg, 0.0429 mmol) was added and the reaction mixture was stirred at 80° C. for 3 h. Upon cooling, EtOAc (15 mL) was added and the mixture was filtered through celite. The filtrate was concentrated to afford the crude material as a viscous brown oil.

Step b: To a mixture of the product of Example 1, step f (445 mg, 0.909 mmol), the crude product of step a (0.857 mmol), and Na₂CO₃ (182 mg, 1.71 mmol) was added dioxane (7.7 mL) and H₂O (0.90 mL), then the suspension was degassed with N₂ for 10 min. (dppf)PdCl₂ (31 mg, 0.0429 mmol) was added and the reaction mixture was stirred at 90° C. for 15 h. Upon cooling, CH₂Cl₂ (20 mL) was added and the mixture was dried over anhyd. MgSO₄, filtered, and concentrated. The residue was purified by silica gel chromatography (100% hexanes to 100% EtOAc) to afford the desired product as a brown solid (438 mg; 90%).

Step c: To a mixture of the product of step b (115 mg, 0.202 mmol) and pyrrolidine (19 μL, 0.222 mmol) in toluene (50 mL) was added 1H-1,2,3-triazole (14 μL, 0.242 mmol) and the reaction mixture was stirred at 100° C. for 24 h. Additional pyrrolidine (2.0 mL, 23.9 mmol) was added, then the reaction mixture was stirred at reflux for 17 h while collecting water via a Dean-Stark trap. Upon cooling, the toluene solution was added over 30 min to a cooled (0° C.) mixture of MeMgBr solution (3 M in Et₂O, 2.33 mL, 6.99 mmol) and THE (10 mL). The reaction mixture was stirred at 0° C. for 1 h, then warmed to r.t. and stirred for 1 h. The reaction mixture was again cooled to 0° C. and sat. aq. NH₄Cl was carefully added followed by H₂O. The aqueous layer was extracted with EtOAc (3×20 mL), then the combined organic layers were washed with NaOH solution (2 N in H₂O, 2×30 mL) and brine, dried over anhyd. MgSO₄, and concentrated to afford a mixture of starting material and the desired intermediate (˜1:1). To a solution of the crude material in CH₂Cl₂ (1.0 mL) was added TFA (1.0 mL). The reaction was stirred for 2 h at r.t. then concentrated. To a solution of the residue in EtOH (1.0 mL) and dioxane (0.5 mL) was added NaOH solution (2 N in H₂O, 1.0 mL) and the reaction mixture stirred at r.t. for 1 h. Sat. aq. NaHCO₃ was added and the mixture was extracted with 10% MeOH in CH₂Cl₂ (3×10 mL), then the combined organic layers were concentrated. Purification by reverse phase HPLC (10 to 70% ACN in H₂O, 0.1% TFA) and lyophilization provided the title compound as a light yellow solid (19 mg, 15%, ˜1:1 d.r.). ¹H NMR (400 MHz, DMSO-d₆) δ 9.35 (p, J=5.8 Hz, 1H), 8.89 (d, J=2.1 Hz, 1H), 8.68 (d, J=2.1 Hz, 1H), 8.41 (t, J=5.4 Hz, 1H), 7.96 (d, J=8.2 Hz, 1H), 7.89 (dd, J=8.2, 1.7 Hz, 1H), 7.71 (d, J=2.0 Hz, 1H), 7.68 (d, J=1.6 Hz, 1H), 7.63 (dd, J=7.7, 2.0 Hz, 1H), 7.33 (d, J=7.8 Hz, 1H), 4.38 (dd, J=5.4, 4.1 Hz, 2H), 3.39 (q, J=5.0 Hz, 2H), 3.35-3.25 (m, 4H), 3.02-2.76 (m, 4H), 2.15-2.04 (m, 2H), 1.99-1.77 (m, 4H), 1.74 (d, J=12.5 Hz, 1H), 1.67 (d, J=12.8 Hz, 1H), 1.52 (s, 3H). ESI MS [M+H]⁺ for C₃₁H₃₄N₅O₂, calcd. 508.3, found 508.2.

### Example 23: 8-(5-{7-Methyl-7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

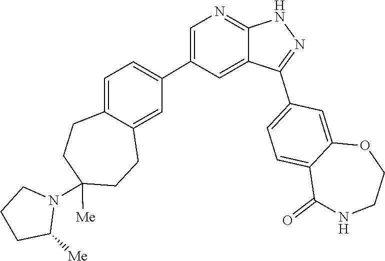

The title compound was prepared in a similar manner to Example 22. ¹H NMR (400 MHz, DMSO-d₆) δ 8.85 (dd, J=2.1, 0.8 Hz, 1H), 8.70-8.53 (m, 2H), 8.38 (t, J=5.4 Hz, 1H), 7.93 (d, J=8.3 Hz, 1H), 7.86 (ddd, J=8.2, 1.7, 0.5 Hz, 1H), 7.67 (s, 1H), 7.64 (d, J=1.7 Hz, 1H), 7.60 (dd, J=7.7, 2.0 Hz, 1H), 7.30 (dd, J=8.0, 1.4 Hz, 1H), 4.34 (dd, J=5.4, 4.1 Hz, 2H), 4.18-4.01 (m, 1H), 3.36 (q, J=5.1 Hz, 2H), 3.33-3.21 (m, 2H), 3.01-2.72 (m, 4H), 2.27-2.11 (m, 1H), 2.10-1.98 (m, 1H), 1.97-1.55 (m, 6H), 1.51 (s, 3H), 1.25 (d, J=6.6 Hz, 3H). ESI MS [M+H]⁺ for C₃₂H₃₆N₅O₂, calcd. 522.3, found 522.2.

### Example 24: 8-(5-{7-Methyl-7-[(2S)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

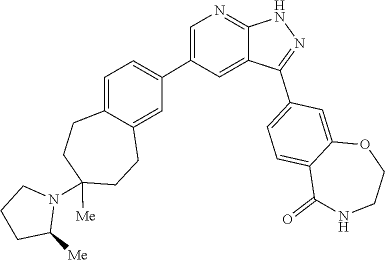

The title compound was prepared in a similar manner to Example 22. ¹H NMR (400 MHz, DMSO-d₆) δ 8.85 (dd, J=2.1, 0.8 Hz, 1H), 8.69-8.61 (m, 2H), 8.38 (t, J=5.3 Hz, 1H), 7.93 (d, J=8.2 Hz, 1H), 7.86 (dd, J=8.1, 1.4 Hz, 1H), 7.67 (s, 1H), 7.64 (d, J=1.7 Hz, 1H), 7.60 (dd, J=7.8, 2.0 Hz, 1H), 7.30 (dd, J=7.7, 1.4 Hz, 1H), 4.34 (dd, J=5.4, 4.1 Hz, 2H), 4.13-4.06 (m, 1H), 3.39-3.22 (m, 4H), 2.97-2.75 (m, 4H), 2.26-2.10 (m, 1H), 2.10-1.97 (m, 1H), 1.97-1.78 (m, 3H), 1.77-1.56 (m, 3H), 1.51 (s, 3H), 1.25 (d, J=6.7 Hz, 3H). ESI MS [M+H]⁺ for C₃₂H₃₆N₅O₂, calcd. 522.3, found 522.2.

### Example 25: 8-(5-{7-[(2R)-2-Ethylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

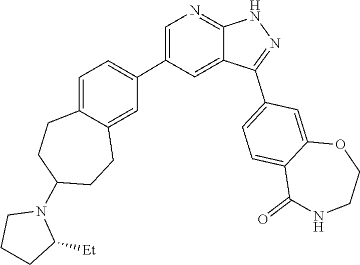

The title compound was prepared in a similar manner to Example 22. ¹H NMR (400 MHz, Chloroform-d) δ 11.90 (br. s, 1H), 8.88 (dd, J=2.0, 0.9 Hz, 1H), 8.51 (d, J=2.0 Hz, 1H), 8.19 (d, J=8.4 Hz, 1H), 7.85-7.77 (m, 1H), 7.70 (td, J=1.1, 0.5 Hz, 1H), 7.44-7.38 (m, 2H), 7.30-7.24 (m, 1H), 6.69 (t, J=5.4 Hz, 1H), 4.58-4.40 (m, 2H), 3.60 (q, J=5.1 Hz, 2H), 3.03-2.82 (m, 5H), 2.82-2.73 (m, 1H), 2.73-2.64 (m, 1H), 2.51 (q, J=8.4 Hz, 1H), 2.25-2.07 (m, 2H), 1.83 (dt, J=11.8, 7.6 Hz, 1H), 1.80-1.51 (m, 4H), 1.47 (td, J=10.5, 9.0, 6.0 Hz, 1H), 1.43-1.35 (m, 1H), 1.24 (dq, J=15.4, 7.6 Hz, 1H), 0.91 (t, J=7.4 Hz, 3H). ESI MS [M+H]⁺ for C₃₂H₃₆N₅O₂, calcd. 522.3, found 522.2.

### Example 26: 7-[5-(3-Cyclopentyl-2,3,4,5-tetrahydro-1H-3-benzazepin-7-yl)-1H-pyrazolo[3,4-b]pyridin-3-yl]-4-methyl-3,4-dihydro-2H-1-benzopyran-4-ol

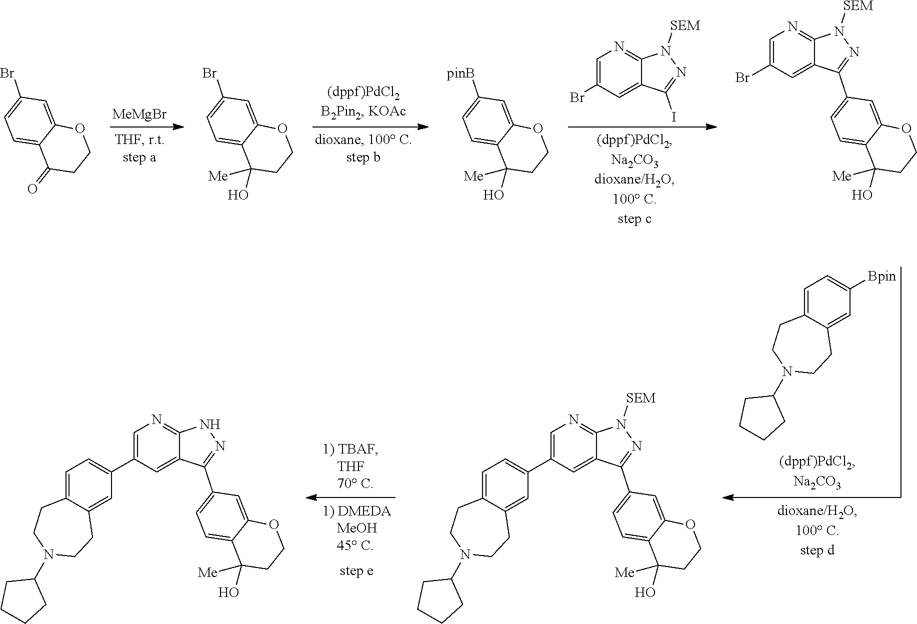

Step a: A solution of 7-bromochromanone (1.00 g, 1.4.40 mmol) in THE (9.8 mL) was added via syringe pump over 40 min. to a solution of methylmagnesium bromide (3.0 M in Et₂O) (3.1 mL, 9.3 mmol) in THE (4.9 mL) at rt. Upon completion of the addition the mixture was stirred at rt for an additional 1 h. The mixture was then poured into ice/sat. NH₄Cl_{(aq)} (50 mL). Product was extracted into EtOAc (3×25 mL) and the combined organic phase was washed with brine (50 mL) and dried (MgSO₄). The material was taken crude into the next step.

Step b: A mixture of the product of step a (4.30 mmol), B₂pin₂ (1.09 g, 4.30 mmol), KOAc (0.844 g, 8.60 mmol), and dioxane (21.5 mL) was sparged with nitrogen for 10 min and then (dppf)PdCl₂ (0.157 g, 0.215 mmol) was added and sparging was continued for 5 min. The mixture was heated at 100° C. for 2 h and then cooled to rt and diluted with EtOAc (100 mL). The mixture was filtered through a pad of celite, the filtrate was concentrated, and the crude material was taken into the next step.

Step c: A solution of the product of Example 1, step e (1.33 g, 2.90 mmol), the product of step b (3.23 mmol) and sodium carbonate (0.615 g, 5.80 mmol) in 9:1 dioxane:H₂O (29 mL) was sparged with nitrogen for 10 min. (dppf)PdCl₂ (0.424 g, 0.580 mmol) was added and sparging was continued for another 5 min. The mixture was stirred at 100° C. overnight and then cooled to rt. CH₂Cl₂ (60 mL) was added and the solution was dried over MgSO₄, concentrated and purified by flash chromatography (SiO₂, 0 to 50% EtOAc in hexanes) to furnish the product as a beige solid (0.741 g; 52%).

Step d: The desired was prepared in a similar manner to step c (136 mg; 54%).

Step e: A mixture of the product of step d (63.8 mg, 0.102 mmol) and 1 M TBAF in THF (1.0 mL) was heated at 70° C. overnight. The mixture was concentrated and then diluted with sat. NaHCO_{3(aq)} (5 mL). Product was extracted into CHCl₃:IPA 9:1 (3×5 mL). The combined organic phase was dried (Na₂SO₄) and concentrated. The residue was taken up in MeOH (1.0 mL) and treated with DMEDA (0.08 mL, 0.77 mmol). The mixture was stirred at 45° C. for 30 min. and then concentrated. The residue was purified by flash chromatography (1 to 10% MeOH/NH_{3(aq)} 10:1 in CH₂Cl₂) to furnish the title compound as an off-white solid (24.7 mg, 49%). ¹H NMR (400 MHz, DMSO-d₆) δ 13.85 (br. s, 1H), 8.85 (d, J=2.1 Hz, 1H), 8.58 (d, J=2.1 Hz, 1H), 7.64 (d, J=1.1 Hz, 2H), 7.59 (d, J=2.0 Hz, 1H), 7.55 (dd, J=7.7, 2.0 Hz, 1H), 7.39 (t, J=1.0 Hz, 1H), 7.26 (d, J=7.8 Hz, 1H), 5.26 (s, 1H), 4.32 (ddd, J=11.3, 7.7, 3.7 Hz, 1H), 4.24 (ddd, J=10.9, 6.9, 3.7 Hz, 1H), 2.99-2.97 (m, 2H), 2.94-2.90 (m, 2H), 2.87 (p, J=7.8 Hz, 1H), 2.72-2.60 (m, 4H), 2.09-1.94 (m, 2H), 1.87-1.75 (m, 2H), 1.69-1.56 (m, 2H), 1.55-1.50 (m, 2H), 1.55 (s, 3H), 1.46-1.33 (m, 2H). ESI MS [M+H]⁺ for C₃₁H₃₅N₄O₂, calcd. 495.3, found 495.2.

### Example 27: 2-[5-(3-Cyclopentyl-2,3,4,5-tetrahydro-1H-3-benzazepin-7-yl)-1H-pyrazolo[3,4-b]pyridin-3-yl]-5-methyl-6,7,8,9-tetrahydro-5H-benzo[7]annulen-5-ol

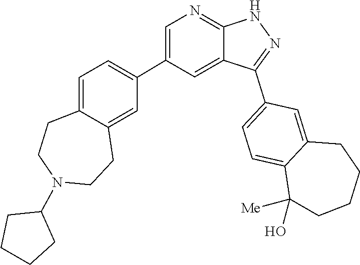

The title compound was prepared in a similar manner to Example 26. ¹H NMR (400 MHz, DMSO-d₆) δ 13.81 (br. s, 1H), 8.84 (d, J=2.1 Hz, 1H), 8.63 (d, J=2.1 Hz, 1H), 7.89 (dd, J=8.2, 1.9 Hz, 1H), 7.82 (d, J=8.2 Hz, 1H), 7.75 (d, J=1.9 Hz, 1H), 7.59 (d, J=2.0 Hz, 1H), 7.55 (dd, J=7.6, 2.0 Hz, 1H), 7.26 (d, J=7.8 Hz, 1H), 5.04 (s, 1H), 3.10 (dd, J=14.2, 7.0 Hz, 1H), 3.02-2.90 (m, 5H), 2.87 (p, J=8.0 Hz, 1H), 2.73-2.58 (m, 4H), 2.00-1.73 (m, 7H), 1.70-1.56 (m, 2H), 1.55-1.36 (m, 5H), 1.52 (s, 3H). ESI MS [M+H]⁺ for C₃₃H₃₉N₄O, calcd. 507.3, found 507.2.

### Example 28: (8-[5-(3-Cyclopentyl-2,3,4,5-tetrahydro-1H-3-benzazepin-7-yl)-1H-pyrazolo[3,4-b]pyridin-3-yl]-5-methyl-2,3,4,5-tetrahydro-1-benzoxepin-5-ol)

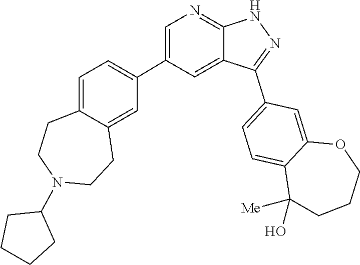

The title compound was prepared in a similar manner to Example 26. ¹H NMR (400 MHz, Chloroform-d) δ 11.49 (br. s, 1H), 8.85 (d, J=2.0 Hz, 1H), 8.49 (d, J=2.1 Hz, 1H), 7.76 (dd, J=8.1, 1.7 Hz, 1H), 7.73 (d, J=7.8 Hz, 1H), 7.65 (d, J=1.4 Hz, 1H), 7.40 (d, J=7.5 Hz, 1H), 7.39-7.37 (m, 1H), 7.26-7.23 (m, 1H), 4.24 (ddd, J=12.0, 6.0, 3.6 Hz, 1H), 3.96 (ddd, J=11.7, 8.5, 2.9 Hz, 1H), 3.04 (ddd, J=13.7, 5.7, 4.0 Hz, 4H), 2.90 (p, J=8.0 Hz, 1H), 2.81-2.72 (m, 4H), 2.48 (s, 1H), 2.24-1.96 (m, 4H), 1.95-1.83 (m, 2H), 1.69 (s, 3H), 1.66-1.41 (m, 6H). ESI MS [M+H]⁺ for C₃₂H₃₇N₄O₂, calcd. 509.3, found 509.2.

### Example 29: 4-Methyl-7-(5-{7-[(2R)-2-Methylpyrrolidin-1-yl]-6,7,8,9-tetrahydro-5H-benzo[7]annulen-2-yl}-1H-pyrazolo[3,4-b]pyridin-3-yl)-3,4-dihydro-2H-1-benzopyran-4-ol

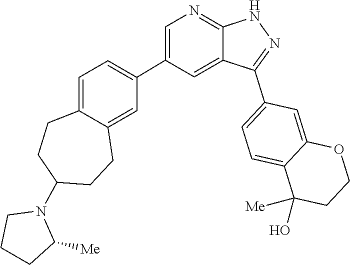

The title compound was prepared in a similar manner to Example 26. ¹H NMR (400 MHz, Chloroform-d) δ 11.03 (br. s, 1H), 8.83 (d, J=2.0 Hz, 1H), 8.47 (d, J=2.0 Hz, 1H), 7.66 (d, J=8.1 Hz, 1H), 7.60 (dd, J=8.1, 1.7 Hz, 1H), 7.46 (d, J=1.7 Hz, 1H), 7.43-7.35 (m, 2H), 7.30-7.22 (m, 1H), 4.42-4.27 (m, 2H), 3.03-2.82 (m, 6H), 2.82-2.69 (m, 1H), 2.50 (q, J=8.4 Hz, 2H), 2.23-2.07 (m, 4H), 1.97 (s, 1H), 1.88 (ddt, J=12.4, 9.0, 6.6 Hz, 1H), 1.71 (s, 3H), 1.66-1.54 (m, 2H), 1.48-1.32 (m, 2H), 1.11 (d, J=6.0 Hz, 3H). ESI MS [M+H]⁺ for C₃₂H₃₇N₄O₂, calcd. 509.3, found 509.2.

### Example 30: 1-Methyl-5-{5-[(7S)-7-(pyrrolidin-1-yl)-6,7,8,9-tetrahydro-5H-benzo[7]annulen-2-yl]-1H-pyrazolo[3,4-b]pyridin-3-yl}-2,3-dihydro-1H-inden-1-ol

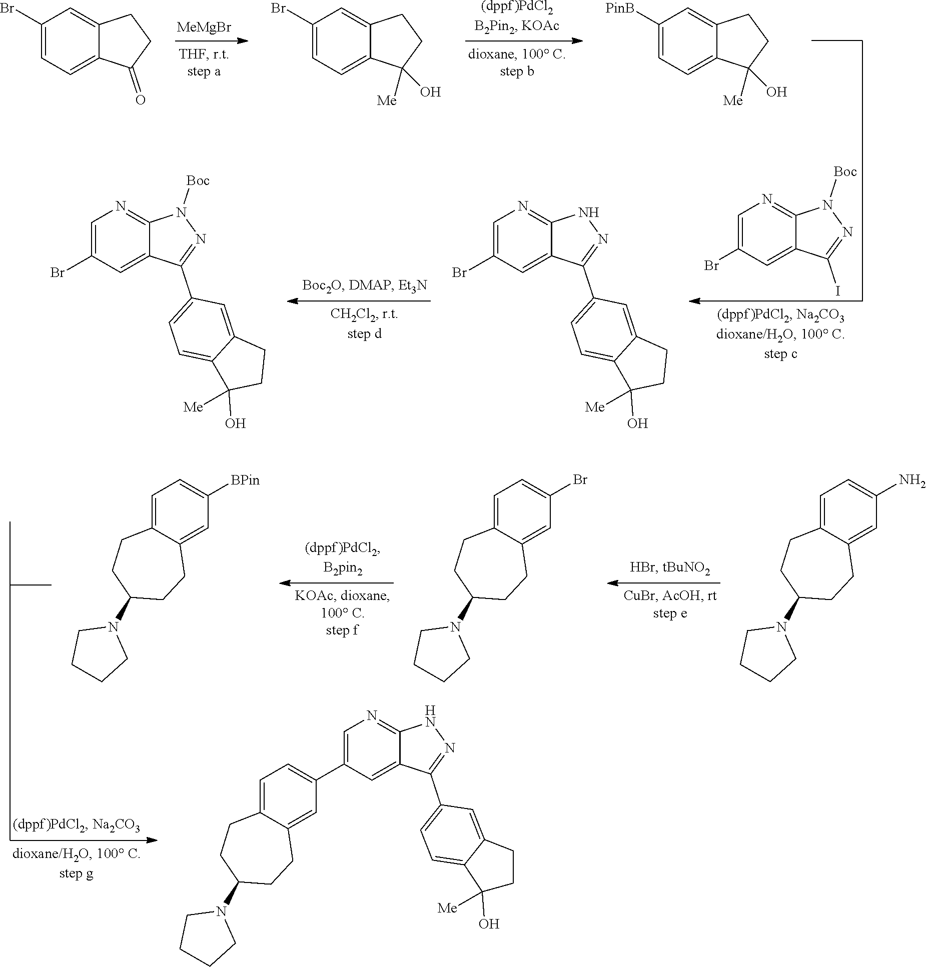

Step a: The desired product was prepared in a similar manner to Example 26, step a (2.01 g; 93%).

Step b: The desired product was prepared in a similar manner to Example 26 step b.

Step c: The desired product was prepared in a similar manner to Example 26, step c (107 mg; 23%).

Step d: To a solution of the product from step c (107 mg, 0.310 mmol), triethylamine (0.09 mL, 0.62 mmol), DMAP (3.9 mg, 0.031 mmol), and CH₂Cl₂ (1.6 mL) at r.t. was added Boc anhydride (71.1 mg, 0.326 mmol). The mixture was stirred at r.t. for 30 min and then concentrated in vacuo. The residue was purified by flash chromatography, EtOAc in hexanes, 0 to 50% to afford the desired product (97 mg; 70%).

Step e: To a mixture of (7S)-6,7,8,9-tetrahydro-7-(1-pyrrolidinyl)-5H-benzocyclohepten-2-amine (2.3 g, 10 mmol), AcOH (33.3 mL), and conc. HBr (2.3 mL, 20 mmol) at rt was added tBuNO₂ (1.3 mL, 11 mmol). The mixture was stirred at rt for 30 min. CuBr (2.9 g, 20 mmol) dissolved in AcOH (20 mL) was added dropwise to the reaction mixture and stirred at rt for 3 h. H₂O (100 mL) was added to dilute the reaction mixture followed by slow addition of 28% wt. NH_{3(aq)} to adjust to pH ˜10-11. The crude product was then extracted with CH₂Cl₂ (2×100 mL). The combined organic phases were dried over Na₂SO₄, concentrated and purified by column chromatography (SiO₂, 0 to 100% CH₂Cl₂/MeOH/7N methanolic NH₃ (90:10:1) in CH₂Cl₂· to afford the desired product as a light brown oil (2.2 g, 75%).

Step f: The desired product was prepared in a similar manner to Example 26, step b.

Step g: The desired product was prepared in a similar manner to Example 26, step c (26 mg, 24%). ¹H NMR (400 MHz, Methanol-d₄) δ 8.76 (d, J=2.1 Hz, 1H), 8.53 (d, J=2.1 Hz, 1H), 7.87-7.83 (m, 1H), 7.83-7.81 (m, 1H), 7.50 (d, J=7.8 Hz, 1H), 7.45 (d, J=2.0 Hz, 1H), 7.42 (dd, J=7.6, 2.0 Hz, 1H), 7.23 (d, J=7.7 Hz, 1H), 3.11 (dt, J=16.1, 6.7 Hz, 1H), 3.01-2.76 (m, 6H), 2.73 (d, J=6.5 Hz, 4H), 2.59 (t, J=10.7 Hz, 1H), 2.34-2.26 (m, 2H), 2.22 (t, J=7.0 Hz, 2H), 1.81 (p, J=3.1 Hz, 4H), 1.56 (s, 3H), 1.40 (p, J=11.4 Hz, 2H). ESI MS [M+H]⁺ for C₃₁H₃₅N₄O, calc. 479.3, found 479.2.

### Example 31: 4-Methyl-7-{5-[(7S)-7-(pyrrolidin-1-yl)-6,7,8,9-tetrahydro-5H-benzo[7]annulen-2-yl]-1H-pyrazolo[3,4-b]pyridin-3-yl}-3,4-dihydro-2H-1-benzopyran-4-ol

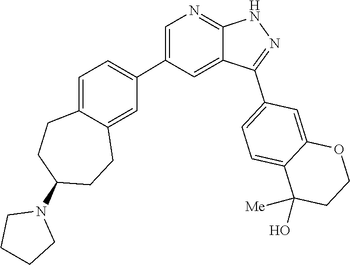

The title compound was prepared in a similar manner to Example 30. ¹H NMR (400 MHz, DMSO-d₆) δ 13.85 (br. s, 1H), 8.85 (d, J=2.1 Hz, 1H), 8.58 (d, J=2.1 Hz, 1H), 7.64 (d, J=1.1 Hz, 2H), 7.60 (d, J=2.0 Hz, 1H), 7.53 (dd, J=7.7, 2.0 Hz, 1H), 7.40 (t, J=1.1 Hz, 1H), 7.26 (d, J=7.8 Hz, 1H), 5.26 (s, 1H), 4.32 (ddd, J=11.3, 7.7, 3.6 Hz, 1H), 4.24 (ddd, J=10.9, 6.8, 3.7 Hz, 1H), 3.20-2.99 (m, 2H), 2.76-2.59 (m, 2H), 2.60-2.53 (m, 4H), 2.01 (qdt, J=11.4, 7.6, 3.4 Hz, 2H), 1.95-1.82 (m, 3H), 1.75-1.69 (m, 4H), 1.66-1.58 (m, 2H), 1.55 (s, 3H). ESI MS [M+H]⁺ for C₃₁H₃₅N₄O₂, calc. 495.3, found 495.2.

### Example 32: 3,3-Dimethyl-6-{5-[7-(pyrrolidin-1-yl)-6,7,8,9-tetrahydro-5H-benzo[7]annulen-2-yl]-1H-pyrazolo[3,4-b]pyridin-3-yl}-2,3-dihydro-1λ⁶,2-benzothiazole-1,1-dione

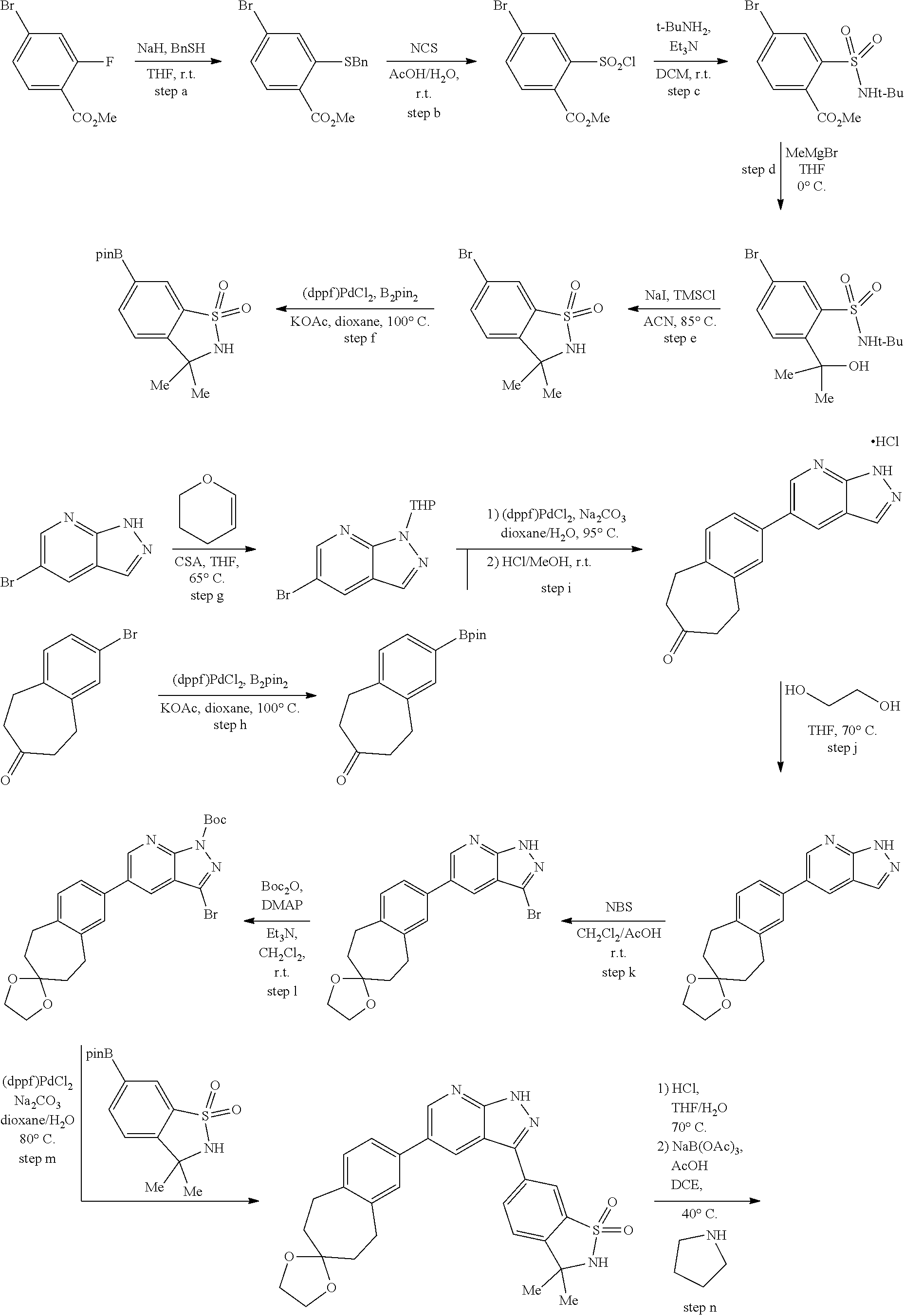

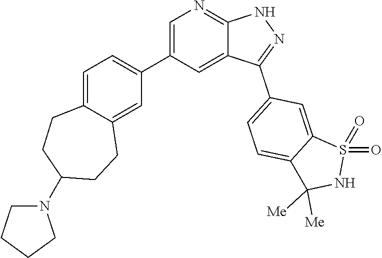 [SMILES: CC1(C)NS(=O)(=O)c2cc(-c3n[nH]c4ncc(-c5ccc6c(c5)CCC(N5CCCC5)CC6)cc34)ccc21]

Step a: To a mixture of NaH (800 g, 20.0 mmol, 60% wt. in oil) in THF (40 mL) at r.t. was added benzyl mercaptan (2.35 mL, 20.0 mmol) dropwise. The reaction mixture was stirred at r.t. for 30 minutes, charged with methyl 4-bromo-2-fluorobenzoate (4.66 g, 20.0 mmol) in one portion, stirred at r.t. for 16 hours, concentrated onto silica gel, and purified by column chromatography (120 g silica gel, hexanes:EtOAc) 0% to 25% gradient (25 minutes) to afford the desired product as a white solid (5.86 g; 87%).

Step b: To a mixture of the product from step a (5.86 g, 17.4 mmol) and 19:1 AcOH:water (87 mL) at r.t. was added NCS (6.96 g, 52.1 mmol) in one portion. The reaction mixture was stirred at r.t. for 1 hour, concentrated, diluted with EtOAc (125 mL), washed with 1:1 sat. NHCO_{3(aq)}:water (2×100 mL), dried over Na₂SO₄, and concentrated to afford the desired product which was used crude in the next step.

Step c: To a mixture of the product from step b (17.4 mmol assumed), Et₃N (12.1 mL, 87.0 mmol), and CH₂Cl₂ (87 mL) at r.t. was added t-BuNH₂ (5.49 mL, 52.2 mmol). The reaction mixture was stirred at r.t. for 3 hours, concentrated onto silica gel, and purified by column chromatography (120 g silica gel, hexanes:EtOAc) 0% to 50% gradient (20 minutes) to afford the desired product as a white solid (5.39 g; 89%; two steps).

Step d: To a mixture of the product from step c (4.96 g, 14.2 mmol) in THE (71 mL) at 0° C. was added MeMgBr (18.9 mL, 56.6 mmol, 3M in Et₂O) dropwise. The reaction mixture was stirred at 0° C. for 45 minutes, stirred at r.t. for 14 hours, quenched with sat. NH₄Cl_{(aq)}, diluted with EtOAc (142 mL), dried over Na₂SO₄, and concentrated to afford the desired product which was used crude in the next step.

Step e: To a mixture of the product from step d (14.2 mmol assumed), NaI (4.09 g, 27.3 mmol), and ACN (68 mL) at r.t. was added chlorotrimethylsilane (3.47 mL, 27.3 mmol). The reaction mixture was stirred at 67° C. for 2 hours, cooled to r.t., quenched with 10% wt. NaHSO_{3(aq)} (142 mL), and diluted with EtOAc (284 mL). The organic phase was dried over Na₂SO₄ and concentrated. The crude material was purified by column chromatography (40 g silica gel, hexanes:EtOAc) 0% to 100% gradient (25 minutes) to afford the desired product as a white solid (2.11 g; 54%; two steps).

Step f: The desired product was prepared in a similar manner to Example 1, step d.

Step g: To a mixture of 5-bromo-1H-pyrazolo[3,4-b]pyridine (19.8 g, 100 mmol), camphorsulfonic acid (2.32 g, 10 mmol), and THF (250 mL) at r.t. was added 3,4-dihydro-2H-pyran (18.3 mL, 200 mmol). The reaction mixture was stirred at 65° C. for 4 hours, cooled to r.t., and quenched with 28% wt. NH_{3(aq)} (10 mL). The mixture was concentrated onto silica gel and purified by column chromatography (330 g silica gel, hexanes:ethyl EtOAc) 0% to 50% gradient (20 minutes) to afford the desired product as a red oil (26.7 g; 95%).

Step h: A mixture of the 2-bromo-5,6,8,9-tetrahydro-7H-benzocyclohepten-7-one (17.9 g, 75.0 mmol), B₂pin₂ (19.1 g, 75.0 mmol), (dppf)PdCl₂ (2.74 g, 3.75 mmol), and KOAc (14.7 g, 150 mmol) was placed under nitrogen. Degassed dioxane (224 mL) was added and the reaction mixture was stirred at 100° C. for 1 hour. The mixture was cooled to r.t. and concentrated. MTBE (375 mL) was added, the mixture filtered through celite, washing with MTBE, and concentrated to afford the desired product which was used crude in the next step.

Step i: A mixture of the product from step g (21.2 g, 75 mmol), the product from step h (75.0 mmol assumed), and (dppf)PdCl₂ (5.49 g, 7.50 mmol) was placed under nitrogen. degassed dioxane (375 mL) and degassed 2M Na₂CO_{3(aq)} (75 mL) were added and the reaction mixture was stirred at 95° C. for 14 hours (or until completion). The mixture was cooled to r.t., concentrated to near dryness, dissolved in ethyl EtOAc (375 mL), dried over Na₂SO₄, and concentrated again. 3M HCl in MeOH (400 mL) was added and the reaction mixture stirred at r.t. for 2 hours and diluted with MTBE (4.00 L) The precipitated solids were collected by filtration, washed with MTBE, and dried under vacuum to afford the desired product as a brown solid (19.4 g; 82%; two steps).

Step j: A mixture of the product from step i (19.4 g, 61.8 mmol), ethylene glycol (17.2 mL, 309 mmol) was stirred at 70° C. for 24 hours, quenched with 28% wt. NH_{3(aq)} (20 mL), and concentrated. EtOAc (500 mL) and water (250 mL) were added and the solids were collected by filtration, washing with EtOAc/water. The organic phase was washed with water (2×250 mL) dried over Na₂SO₄, concentrated, and combined with the previously collected solids. The crude material was purified by column chromatography (330 g silica gel, CH₂Cl₂:MeOH) 0% to 3% gradient (20 minutes); 3% to 5% gradient (10 minutes) to afford the desired product as an orange solid (14.8 g; 75%).

Step k: To a mixture of the product from step j (14.8 g, 46.1 mmol) and 2:1 CH₂Cl₂:AcOH (138 mL) at r.t. was added NBS (8.62 g, 48.5 mmol). The reaction mixture was stirred at r.t. for 14 hours, concentrated onto silica gel, and purified by column chromatography (330 g silica gel, CH₂Cl₂:MeOH) 0% to 5% gradient (15 minutes); 5% to 7.5% gradient (5 minutes) to afford the desired product as a brown solid (21.4 g; 74.5% wt.; balance succinimide). If pure, 15.9 g (86% yield).

Step l: To a mixture of the product from step k (21.4 g, 39.7 mmol, 74.5% wt.), DMAP (486 mg, 3.97 mmol), Et₃N (26.4 mL, 189 mmol), and CH₂Cl₂ (199 mL) at r.t. was added di-tert-butyl dicarbonate (21.7 g, 99.4 mmol) in one portion. The reaction mixture was stirred at r.t. for 1 hour, concentrated onto silica gel, and purified by column chromatography (330 g silica gel, hexanes:EtOAc) 0% to 50% gradient (25 minutes) to afford the desired product as a white solid (18.2 g; 77.4% wt.; balance N-Boc-succinimide). If pure, 14.1 g (71% yield).

Step m: The desired product was prepared in a similar manner to Example 7, step c. (110 mg; 53%).

Step n: A mixture of the product from step m (110 mg, 0.213 mmol), HCl (426 μL, 0.426 mmol, 1M in water), and THE (1.1 mL) was stirred at 70° C. for 1 hour. The mixture was cooled to r.t., neutralized with sat. NaHCO_{3(aq)}, washed with brine (1.1 mL), concentrated, diluted with CH₂Cl₂ (10 mL), dried over Na₂SO₄, and again concentrated. Pyrrolidine (21 μL, 0.26 mmol), AcOH (12 μL, 0.21 mmol), and DCE (1.1 mL) were added, followed by NaBH(OAc)₃ (67 mg, 0.32 mmol). The reaction mixture was stirred at r.t. for 4 hours, quenched with 1:1 sat. NaHCO_{3(aq)}:water (8.0 mL), and extracted with 4:1 CH₂Cl₂:IPA (1×25 mL). The organic phase was dried over Na₂SO₄ and concentrated. The crude material was purified by HPLC ((H₂O/ACN)+0.1% TFA) 5% to 95% gradient (30 minutes) to afford the desired product as a light yellow solid (106 mg; 79%). ¹H NMR (400 MHz, DMSO-d₆) δ 8.87 (d, J=2.1 Hz, 1H), 8.70 (d, J=2.1 Hz, 1H), 8.44 (dd, J=8.2, 1.6 Hz, 1H), 8.32 (dd, J=1.6, 0.6 Hz, 1H), 8.04 (s, 1H), 7.85 (dd, J=8.2, 0.6 Hz, 1H), 7.60 (d, J=2.0 Hz, 1H), 7.54 (dd, J=7.7, 2.0 Hz, 1H), 7.25 (d, J=7.8 Hz, 1H), 3.19-2.96 (m, 2H), 2.76-2.61 (m, 2H), 2.61-2.43 (m, 5H), 2.03-1.80 (m, 2H), 1.80-1.66 (m, 4H), 1.60 (s, 8H). ESI MS [M+H]⁺ for C₃₀H₃₄N₅O₂S, calcd. 528.2, found 528.3.

### Example 33: 3,3-Dimethyl-6-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3-dihydro-1λ⁶,2-benzothiazole-1,1-dione

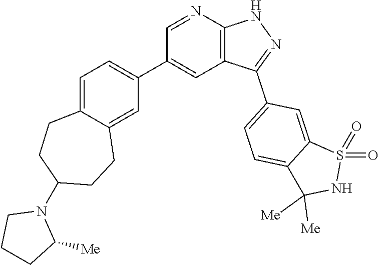 [SMILES: C[C@@H]1CCCN1C1CCc2ccc(-c3cnc4[nH]nc(-c5ccc6c(c5)S(=O)(=O)NC6(C)C)c4c3)cc2CC1]

The title compound was prepared in a similar manner to Example 32. ¹H NMR (400 MHz, DMSO-d₆) δ 8.88 (dd, J=2.0, 0.8 Hz, 1H), 8.71 (d, J=0.6 Hz, 1H), 8.44 (ddd, J=8.2, 1.6, 0.6 Hz, 1H), 8.32 (dt, J=1.5, 0.7 Hz, 1H), 8.04 (s, 1H), 7.85 (d, J=8.1 Hz, 1H), 7.62 (t, J=2.6 Hz, 1H), 7.56 (dd, J=7.7, 2.0 Hz, 1H), 7.27 (dd, J=7.7, 1.3 Hz, 1H), 3.01-2.77 (m, 5H), 2.77-2.65 (m, 2H), 2.44 (q, J=8.2 Hz, 1H), 2.06-1.95 (m, 2H), 1.87-1.77 (m, 1H), 1.67-1.50 (m, 8H), 1.44 (t, J=12.3 Hz, 1H), 1.34-1.21 (m, 2H), 1.02 (d, J=6.0 Hz, 3H). ESI MS [M+H]⁺ for C₃₁H₃₆N₅O₂S, calcd. 542.3, found 542.2.

### Example 34: 6-(5-{7-[(2R)-2-(Hydroxymethyl)pyrrolidin-1-yl]-6,7,8,9-tetrahydro-5H-benzo[7]annulen-2-yl}-1H-pyrazolo[3,4-b]pyridin-3-yl)-3,3-dimethyl-2,3-dihydro-1λ⁶,2-benzothiazole-1,1-dione

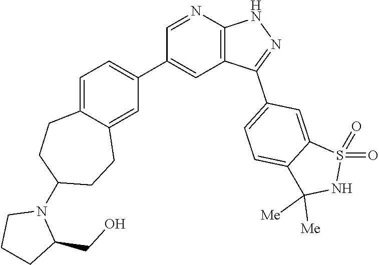 [SMILES: CC1(C)NS(=O)(=O)c2cc(-c3n[nH]c4ncc(-c5ccc6c(c5)CCC(N5CCC[C@@H]5CO)CC6)cc34)ccc21]

The title compound was prepared in a similar manner to Example 32. ¹H NMR (400 MHz, DMSO-d₆) δ 8.88 (d, J=1.9 Hz, 1H), 8.71 (d, J=2.0 Hz, 1H), 8.44 (dd, J=8.1, 1.6 Hz, 1H), 8.32 (dt, J=1.5, 0.7 Hz, 1H), 8.04 (s, 1H), 7.85 (dd, J=8.1, 0.7 Hz, 1H), 7.64-7.60 (m, 1H), 7.56 (dd, J=7.7, 2.0 Hz, 1H), 7.27 (d, J=7.8 Hz, 1H), 3.38-3.34 (m, 1H), 3.16-3.09 (m, 1H), 2.99-2.81 (m, 4H), 2.80-2.65 (m, 3H), 2.54-2.51 (m, 1H), 2.12-2.02 (m, 2H), 1.72-1.53 (m, 10H), 1.46-1.25 (m, 2H). ESI MS [M+H]⁺ for C₃₁H₃₆N₅O₃S, calcd. 558.3, found 558.2.

### Example 35: 3,3-Dimethyl-8-{5-[7-(pyrrolidin-1-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

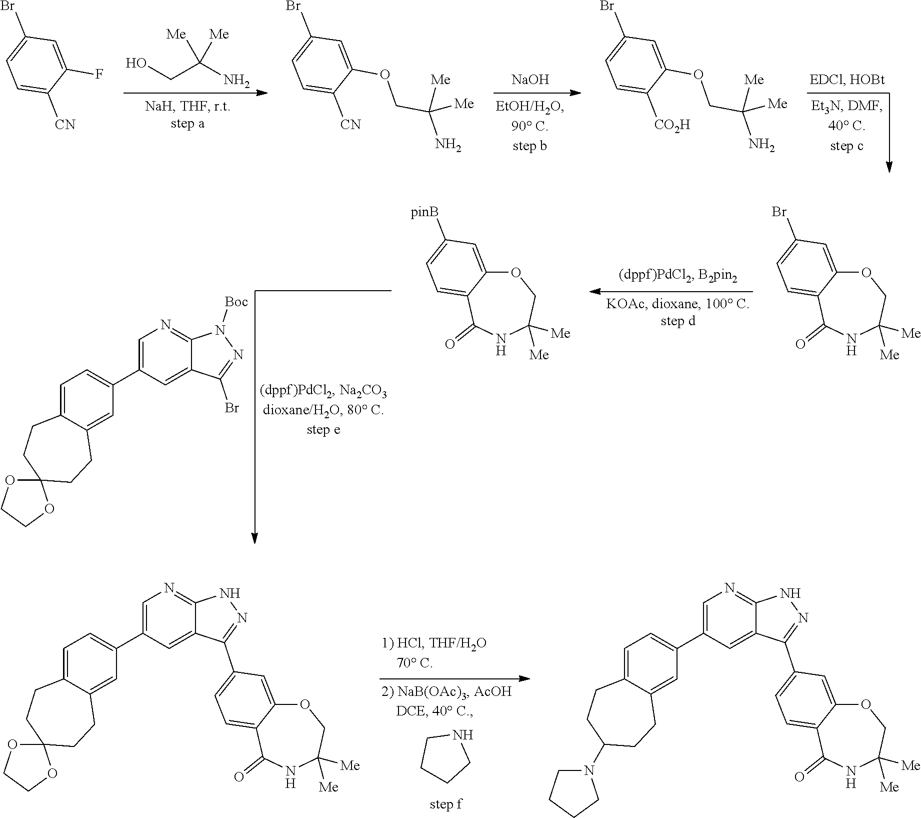

Step a: To a mixture of 4-bromo-2-fluorobenzonitrile (2.00 g, 10.0 mmol), 2-amino-2-methyl-1-propanol (954 μL, 10.0 mmol), and THF (20 mL) at 0° C. was added NaH (400 g, 10.0 mmol, 60% wt. in oil) in one portion. The reaction mixture was stirred at 0° C. for 1 hour, stirred at r.t. for 14 hours, concentrated onto silica gel, and purified by column chromatography (80 g silica gel, CH₂Cl₂:MeOH) 0% to 20% gradient (30 minutes) to afford the desired product as a yellow solid (1.82 g; 68%).

Step b: A mixture of the product from step a (1.82 g, 6.76 mmol), NaOH (848 mg, 21.2 mmol), and 4:1 EtOH:water (14 mL) was stirred at 90° C. for 14 hours, cooled to r.t., and concentrated to remove EtOH. The resultant mixture was adjusted to pH ˜4 by addition of 2M HCl_{(aq)} (˜2.5 eq). The formed solids were collected by filtration, washed with water, and dried to afford the desired product as a light brown solid (1.93 g; 99%).

Step c: To a mixture of the product from step b (1.93 g, 6.70 mmol), HOBt hydrate (1.13 g, 7.37 mmol), Et₃N (3.73 mL, 26.8 mmol), and DMF (33 mL) at r.t. was added 1-(3-dimethylaminopropyl)-3-ethylcarbodiimide hydrochloride (1.93 g, 10.1 mmol) in one portion. The reaction mixture was stirred at 40° C. for 3 days, diluted with EtOAc (125 mL) washed with 9:1 water:brine (4×100 mL), dried over Na₂SO₄, and concentrated. The crude material was purified by column chromatography (40 g silica gel, hexanes:EtOAc) 0% to 100% gradient (25 minutes) to afford the desired product as a yellow solid (1.24 g; 69%).

Step d: The desired product was prepared in a similar manner to Example 1, step d.

Step e: The desired product was prepared in a similar manner to Example 7, step c (103 mg; 50%).

Step f: The desired product was prepared in a similar manner to Example 32, step n (37 mg; 37%). ¹H NMR (400 MHz, DMSO-d₆) δ 8.86 (d, J=2.1 Hz, 1H), 8.67 (d, J=2.1 Hz, 1H), 8.35 (d, J=8.4 Hz, 1H), 8.24 (s, 1H), 7.86 (dd, J=8.5, 1.8 Hz, 1H), 7.69 (d, J=1.8 Hz, 1H), 7.61 (d, J=2.0 Hz, 1H), 7.54 (dd, J=7.6, 2.0 Hz, 1H), 7.25 (d, J=7.8 Hz, 1H), 4.18 (s, 2H), 3.18-2.98 (m, 2H), 2.77-2.61 (m, 2H), 2.61-2.45 (m, 5H), 2.01-1.79 (m, 2H), 1.79-1.65 (m, 4H), 1.65-1.49 (m, 2H), 1.27 (s, 6H). ESI MS [M+H]⁺ for C₃₂H₃₆N₅O₂, calcd. 522.3, found 522.3.

### Example 36: 8-(5-{7-[(2R)-2-Methylpyrrolidin-1-yl]-6,7,8,9-tetrahydro-5H-benzo[7]annulen-2-yl}-1H-pyrazolo[3,4-b]pyridin-3-yl)-4,5-dihydro-2H-spiro[1,4-benzoxazepine-3,1′-cyclopropan]-5-one

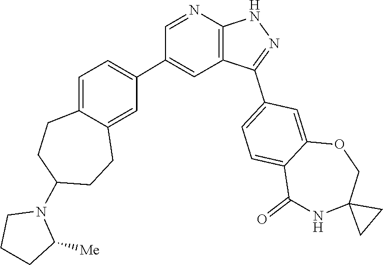

The title compound was prepared in a similar manner to Example 35. ¹H NMR (400 MHz, DMSO-d₆) δ 14.04 (s, 1H), 8.87 (d, J=2.0 Hz, 1H), 8.70 (s, 1H), 8.68 (d, J=2.0 Hz, 1H), 7.91 (dd, J=8.2, 1.5, 0.6 Hz, 1H), 7.88 (d, J=8.2 Hz, 1H), 7.69 (d, J=1.5 Hz, 1H), 7.62 (t, J=2.3 Hz, 1H), 7.55 (dd, J=7.7, 2.0 Hz, 1H), 7.27 (d, J=7.2 Hz, 1H), 4.27 (s, 2H), 3.01-2.76 (m, 5H), 2.76-2.67 (m, 2H), 2.44 (q, J=8.3 Hz, 1H), 2.06-1.94 (m, 2H), 1.88-1.76 (m, 1H), 1.68-1.49 (m, 2H), 1.49-1.37 (m, 1H), 1.35-1.20 (m, 2H), 1.02 (d, J=5.9 Hz, 3H), 0.79 (d, J=7.1 Hz, 4H). ESI MS [M+H]⁺ for C₃₃H₃₆N₅O₂, calcd. 534.3, found 534.3.

### Example 37: 8-(5-{7-[(2R)-2-Methylpyrrolidin-1-yl]-6,7,8,9-tetrahydro-5H-benzo[7]annulen-2-yl}-1H-pyrazolo[3,4-b]pyridin-3-yl)-4,5-dihydro-2H-spiro[1,4-benzoxazepine-3,1′-cyclobutan]-5-one

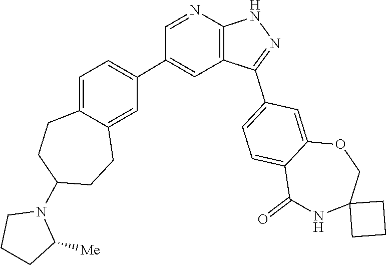

The title compound was prepared in a similar manner to Example 35. ¹H NMR (400 MHz, DMSO-d₆) δ 8.86 (d, J=2.0 Hz, 1H), 8.70 (s, 1H), 8.67 (d, J=2.1 Hz, 1H), 8.19 (d, J=8.4 Hz, 1H), 7.85 (ddd, J=8.4, 1.8, 0.5 Hz, 1H), 7.70 (d, J=1.7 Hz, 1H), 7.62 (t, J=2.3 Hz, 1H), 7.55 (dd, J=7.7, 2.0 Hz, 1H), 7.27 (d, J=7.0 Hz, 1H), 4.40 (s, 2H), 3.02-2.77 (m, 5H), 2.77-2.65 (m, 2H), 2.44 (q, J=8.2 Hz, 1H), 2.25-2.14 (m, 2H), 2.13-2.05 (m, 2H), 2.05-1.95 (m, 2H), 1.87-1.73 (m, 3H), 1.68-1.49 (m, 2H), 1.49-1.39 (m, 1H), 1.34-1.21 (m, 2H), 1.03 (d, J=6.0 Hz, 3H). ESI MS [M+H]⁺ for C₃₄H₃₈N₅O₂, calcd. 548.3, found 548.3.

### Example 38: 3,3-Dimethyl-6-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3-dihydro-1H-isoindol-1-one

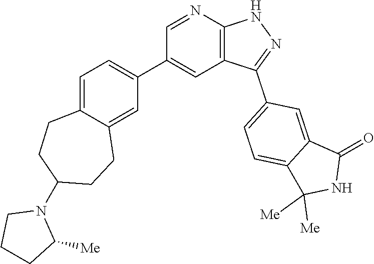 [SMILES: C[C@@H]1CCCN1C1CCc2ccc(-c3cnc4[nH]nc(-c5ccc6c(c5)C(=O)NC6(C)C)c4c3)cc2CC1]

The title compound was prepared in a similar manner to Example 35. ¹H NMR (400 MHz, DMSO-d₆) δ 13.96 (s, 1H), 8.86 (d, J=1.5 Hz, 1H), 8.78 (s, 1H), 8.63 (dd, J=2.1, 1.0 Hz, 1H), 8.33 (dd, J=7.9, 1.6 Hz, 1H), 8.18 (dd, J=1.6, 0.7 Hz, 1H), 7.79 (d, J=7.9 Hz, 1H), 7.60 (t, J=2.4 Hz, 1H), 7.54 (dd, J=7.7, 2.0 Hz, 1H), 7.26 (d, J=7.4 Hz, 1H), 3.02-2.76 (m, 5H), 2.76-2.63 (m, 2H), 2.44 (q, J=8.2 Hz, 1H), 2.07-1.93 (m, 2H), 1.88-1.76 (m, 1H), 1.69-1.37 (m, 9H), 1.35-1.19 (m, 2H), 1.02 (d, J=6.0 Hz, 3H). ESI MS [M+H]⁺ for C₃₂H₃₆N₅O, calcd. 506.3, found 506.3.

### Example 39: 3,3-Dimethyl-8-(5-{2-[(2S)-2-methylpyrrolidin-1-yl]-2,3-dihydro-1H-inden-5-yl}-1H-pyrazolo[3,4-b]pyridin-3-yl)-2,3,4,5-tetrahydro-1,4-benzoxazepin-5-one

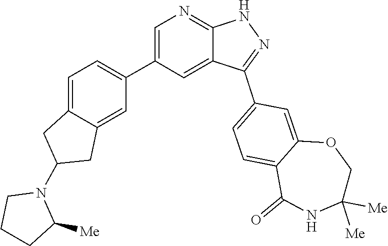

The title compound was prepared in a similar manner to Example 35. ¹H NMR (400 MHz, DMSO-d₆) δ 9.73 (s, 1H), 8.84 (d, J=2.1 Hz, 1H), 8.65 (dd, J=2.2, 0.6 Hz, 1H), 8.32 (dt, J=8.5, 0.5 Hz, 1H), 8.21 (s, 1H), 7.82 (ddd, J=8.5, 1.8, 0.6 Hz, 1H), 7.77-7.72 (m, 1H), 7.71-7.64 (m, 2H), 7.40 (dd, J=7.9, 5.9 Hz, 1H), 4.30 (dt, J=11.6, 7.9 Hz, 1H), 3.93-3.25 (m, 6H), 3.18 (td, J=14.9, 6.2 Hz, 3H), 2.23 (dq, J=14.1, 7.3 Hz, 1H), 1.94 (ddq, J=28.1, 13.9, 7.2, 6.7 Hz, 2H), 1.64 (dq, J=14.1, 7.3 Hz, 1H), 1.39 (d, J=6.6 Hz, 3H), 1.23 (s, 6H). ESI MS [M+H]⁺ for C₃₁H₃₄N₅O₂, calcd. 508.3, found 508.2.

### Example 40: 7-Methyl-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

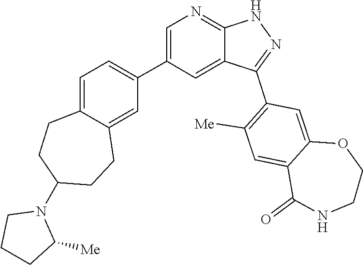 [SMILES: Cc1cc2c(cc1-c1n[nH]c3ncc(-c4ccc5c(c4)CCC(N4CCC[C@H]4C)CC5)cc13)OCCNC2=O]

The title compound was prepared in a similar manner to Example 35. ¹H NMR (400 MHz, DMSO-d₆) δ 8.86 (d, J=2.1 Hz, 1H), 8.39 (t, J=5.4 Hz, 1H), 8.27 (d, J=2.1 Hz, 1H), 7.78 (d, J=0.8 Hz, 1H), 7.54 (s, 1H), 7.48 (d, J=7.5 Hz, 1H), 7.25-7.19 (m, 2H), 4.32 (dd, J=5.3, 4.2 Hz, 2H), 3.41-3.35 (m, 2H), 2.97-2.75 (m, 4H), 2.75-2.63 (m, 2H), 2.42 (d, J=8.5 Hz, 1H), 2.39 (d, J=0.7 Hz, 3H), 2.34-2.31 (m, 1H), 2.03-1.91 (m, 2H), 1.88-1.74 (m, 1H), 1.68-1.49 (m, 2H), 1.46-1.35 (m, 1H), 1.33-1.18 (m, 2H), 1.01 (d, J=6.0 Hz, 3H). ESI MS [M+H]⁺ for C₃₂H₃₆N₅O₂, calcd. 522.3, found 522.3.

### Example 41: 9-Methyl-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

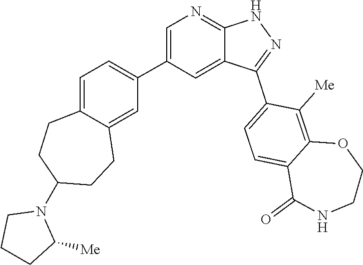 [SMILES: Cc1c(-c2n[nH]c3ncc(-c4ccc5c(c4)CCC(N4CCC[C@H]4C)CC5)cc23)ccc2c1OCCNC2=O]

The title compound was prepared in a similar manner to Example 35. ¹H NMR (400 MHz, DMSO-d₆) δ 8.83 (d, J=2.1 Hz, 1H), 8.37 (t, J=5.5 Hz, 1H), 8.23 (d, J=2.1 Hz, 1H), 7.59 (d, J=8.0 Hz, 1H), 7.53 (d, J=2.2 Hz, 1H), 7.47 (dd, J=7.7, 2.0 Hz, 1H), 7.42 (d, J=8.0 Hz, 1H), 7.21 (d, J=7.7 Hz, 1H), 4.33 (d, J=5.3 Hz, 2H), 3.55-3.23 (m, 2H), 2.96-2.62 (m, 7H), 2.42 (q, J=8.3 Hz, 1H), 2.33 (s, 3H), 2.03-1.93 (d, J=13.1 Hz, 2H), 1.86-1.74 (m, 1H), 1.68-1.50 (m, 2H), 1.46-1.36 (m, 1H), 1.32-1.20 (m, 2H), 1.01 (d, J=6.0 Hz, 3H). ESI MS [M+H]⁺ for C₃₂H₃₆N₅O₂, calcd. 522.3, found 522.3.

### Example 42: 8-{5-[7-(Cyclopentylamino)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

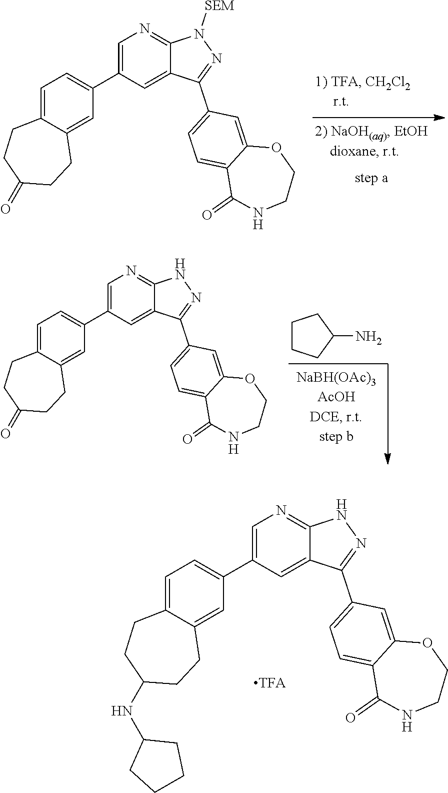 [SMILES: C[Si](C)(C)CCOCn1nc(-c2ccc3c(c2)OCCNC3=O)c2cc(-c3ccc4c(c3)CCC(=O)CC4)cnc21 ; O=C1CCc2ccc(-c3cnc4[nH]nc(-c5ccc6c(c5)OCCNC6=O)c4c3)cc2CC1 ; NC1CCCC1 ; O=C1NCCOc2cc(-c3n[nH]c4ncc(-c5ccc6c(c5)CCC(NC5CCCC5)CC6)cc34)ccc21]

Step a: To a solution of the product of Example 22, step b (635 mg, 1.12 mmol) in CH₂Cl₂ (2.8 mL) was added TFA (2.8 mL). The reaction was stirred for 2 h at r.t. then concentrated. To a suspension of the residue in EtOH (5.6 mL) was added NaOH solution (2 N in H₂O, 5.6 mL). Dioxane (10 mL) was added and the reaction mixture stirred at r.t. for 2 h. Sat. aq. NaHCO₃ was added and the mixture was extracted with 10% MeOH in CH₂Cl₂ (3×15 mL), then the combined organic layers were concentrated to provide the product as a yellow solid (230 mg; 47%).

Step b: To a mixture of the product of step a (58 mg, 0.132 mmol) and cyclopentylamine (14 μL, 0.139 mmol) in 1,2-dichloroethane (1.4 mL) was added AcOH (7 μL, 0.132 mmol) and the mixture stirred at r.t. for 30 min, then NaBH(OAc)₃ (63 mg, 0.296 mmol) was added. The reaction mixture was stirred at r.t. for 15 h and carefully quenched with H₂O then sat. aq. NaHCO₃. The mixture was extracted with 10% MeOH in CH₂Cl₂ (3×10 mL), then the combined organic layers were concentrated. Purification by reverse phase HPLC (10 to 70% ACN in H₂O, 0.1% TFA) and lyophilization provided the title compound as a white solid (16 mg, 19%). ¹H NMR (400 MHz, CD₃OD) δ 8.82 (d, J=2.0 Hz, 1H), 8.62 (d, J=2.1 Hz, 1H), 8.02 (d, J=8.2 Hz, 1H), 7.86 (dd, J=8.2, 1.7 Hz, 1H), 7.72 (d, J=1.6 Hz, 1H), 7.57 (d, J=2.0 Hz, 1H), 7.54 (dd, J=7.7, 2.0 Hz, 1H), 7.33 (d, J=7.7 Hz, 1H), 4.46 (dd, J=5.1, 4.4 Hz, 2H), 3.80 (p, J=7.4 Hz, 1H), 3.56-3.46 (m, 3H), 3.11-3.04 (m, 1H), 3.04-2.93 (m, 3H), 2.53-2.40 (m, 2H), 2.25-2.08 (m, 2H), 1.92-1.78 (m, 2H), 1.78-1.56 (m, 4H), 1.56-1.38 (m, 2H). ESI MS [M+H]⁺ for C₃₁H₃₄N₅O₂, calcd. 508.3, found 508.2.

### Example 43: 8-(5-{7-[(2S)-2-Methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

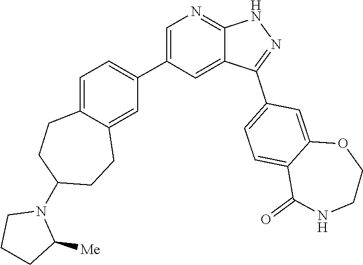 [SMILES: C[C@H]1CCCN1C1CCc2ccc(-c3cnc4[nH]nc(-c5ccc6c(c5)OCCNC6=O)c4c3)cc2CC1]

The title compound was prepared in a similar manner to Example 42. ¹H NMR (400 MHz, Methanol-d₄) δ 8.73 (dd, J=2.1, 1.0 Hz, 1H), 8.49 (dd, J=2.1, 1.1 Hz, 1H), 7.97 (dd, J=8.2, 0.4 Hz, 1H), 7.77 (dd, J=8.2, 1.7 Hz, 1H), 7.63 (dd, J=1.6, 0.4 Hz, 1H), 7.48 (t, J=2.2 Hz, 1H), 7.44 (ddd, J=7.7, 2.1, 0.9 Hz, 1H), 7.26 (d, J=7.8 Hz, 1H), 4.49-4.37 (m, 2H), 3.85-3.63 (m, 2H), 3.54-3.48 (m, 2H), 3.48-3.38 (m, 1H), 3.30-3.18 (m, 1H), 3.11-2.82 (m, 4H), 2.43-2.33 (m, 2H), 2.29 (dq, J=13.6, 6.9 Hz, 1H), 2.02 (p, J=7.4 Hz, 2H), 1.74 (dq, J=13.0, 7.9 Hz, 1H), 1.68-1.50 (m, 2H), 1.47 (d, J=6.4 Hz, 3H). ESI MS [M+H]⁺ for C₃₁H₃₄N₅O₂, calcd. 508.3, found 508.2.

### Example 44: 8-(5-{7-[(2R)-2-Methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

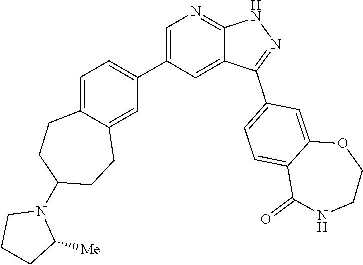 [SMILES: C[C@@H]1CCCN1C1CCc2ccc(-c3cnc4[nH]nc(-c5ccc6c(c5)OCCNC6=O)c4c3)cc2CC1]

The title compound was prepared in a similar manner to Example 42. ¹H NMR (400 MHz, Methanol-d₄) δ 8.73 (dd, J=2.1, 1.0 Hz, 1H), 8.49 (dd, J=2.1, 1.1 Hz, 1H), 7.97 (dd, J=8.2, 0.3 Hz, 1H), 7.77 (dd, J=8.2, 1.7 Hz, 1H), 7.63 (d, J=1.6 Hz, 1H), 7.48 (t, J=2.2 Hz, 1H), 7.44 (dd, J=7.7, 1.8 Hz, 1H), 7.26 (d, J=7.8 Hz, 1H), 4.44 (dd, J=5.5, 4.1 Hz, 2H), 3.85-3.63 (m, 2H), 3.52-3.48 (m, 2H), 3.48-3.40 (m, 1H), 3.28-3.19 (m, 1H), 3.09-2.85 (m, 4H), 2.43-2.33 (m, 2H), 2.29 (dq, J=13.6, 6.9 Hz, 1H), 2.02 (p, J=7.4 Hz, 2H), 1.74 (dq, J=13.0, 7.9 Hz, 1H), 1.68-1.50 (m, 2H), 1.47 (d, J=6.4 Hz, 3H). ESI MS [M+H]⁺ for C₃₁H₃₄N₅O₂, calcd. 508.3, found 508.2.

### Example 45: 8-[5-(7-{[(3S)—Oxolan-3-yl]amino}-6,7,8,9-tetrahydro-5H-benzo[7]annulen-2-yl)-1H-pyrazolo[3,4-b]pyridin-3-yl]-2,3,4,5-tetrahydro-1,4-benzoxazepin-5-one

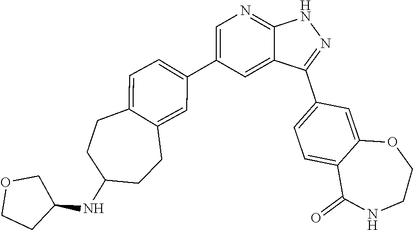 [SMILES: O=C1NCCOc2cc(-c3n[nH]c4ncc(-c5ccc6c(c5)CCC(N[C@H]5CCOC5)CC6)cc34)ccc21]

The title compound was prepared in a similar manner to Example 42. ¹H NMR (400 MHz, DMSO-d₆) δ 8.89 (d, J=2.0 Hz, 1H), 8.69 (d, J=2.1 Hz, 1H), 8.64-8.46 (m, 2H), 8.41 (t, J=5.4 Hz, 1H), 7.96 (dd, J=8.2, 0.4 Hz, 1H), 7.90 (dd, J=8.2, 1.7 Hz, 1H), 7.71-7.66 (m, 2H), 7.63 (dd, J=7.7, 1.9 Hz, 1H), 7.32 (d, J=7.8 Hz, 1H), 4.38 (dd, J=5.4, 4.1 Hz, 2H), 4.16-4.00 (m, 1H), 3.95 (td, J=8.3, 5.4 Hz, 1H), 3.85 (d, J=4.9 Hz, 2H), 3.71-3.64 (m, 1H), 3.58-3.43 (m, 1H), 3.39 (q, J=5.0 Hz, 2H), 3.10-2.76 (m, 4H), 2.43-2.25 (m, 3H), 2.02-1.92 (m, 1H), 1.36 (p, J=12.6, 12.0 Hz, 2H). ESI MS [M+H]⁺ for C₃₀H₃₂N₅O₃, calcd. 510.2, found 510.2.

### Example 46: 8-[5-(7-{[(3R)—Oxolan-3-yl]amino}-6,7,8,9-tetrahydro-5H-benzo[7]annulen-2-yl)-1H-pyrazolo[3,4-b]pyridin-3-yl]-2,3,4,5-tetrahydro-1,4-benzoxazepin-5-one

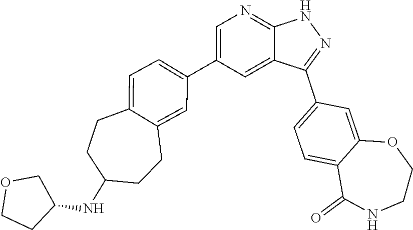 [SMILES: O=C1NCCOc2cc(-c3n[nH]c4ncc(-c5ccc6c(c5)CCC(N[C@@H]5CCOC5)CC6)cc34)ccc21]

The title compound was prepared in a similar manner to Example 42. ¹H NMR (400 MHz, DMSO-d₆) δ 8.89 (d, J=2.1 Hz, 1H), 8.69 (d, J=2.1 Hz, 1H), 8.65-8.49 (m, 2H), 8.41 (t, J=5.4 Hz, 1H), 7.96 (d, J=8.2 Hz, 1H), 7.90 (dd, J=8.2, 1.7 Hz, 1H), 7.68 (d, J=1.5 Hz, 2H), 7.63 (dd, J=7.8, 2.0 Hz, 1H), 7.32 (d, J=7.8 Hz, 1H), 4.38 (dd, J=5.3, 4.1 Hz, 2H), 4.15-4.03 (m, 1H), 3.95 (td, J=8.4, 5.3 Hz, 1H), 3.85 (d, J=5.0 Hz, 2H), 3.68 (ddd, J=8.7, 7.8, 7.1 Hz, 1H), 3.55-3.43 (m, 1H), 3.39 (q, J=5.0 Hz, 2H), 3.04-2.82 (m, 4H), 2.42-2.24 (m, 3H), 2.03-1.91 (m, 1H), 1.36 (p, J=12.1 Hz, 2H). ESI MS [M+H]⁺ for C₃₀H₃₂N₅O₃, calcd. 510.2, found 510.2.

### Example 47: 8-{5-[7-(Morpholin-4-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

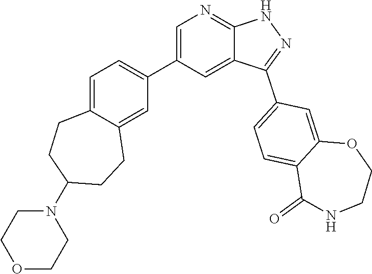 [SMILES: O=C1NCCOc2cc(-c3n[nH]c4ncc(-c5ccc6c(c5)CCC(N5CCOCC5)CC6)cc34)ccc21]

The title compound was prepared in a similar manner to Example 42. ¹H NMR (400 MHz, Methanol-d₄) δ 8.78 (d, J=2.0 Hz, 1H), 8.56 (dd, J=2.1, 0.9 Hz, 1H), 8.00 (d, J=8.2 Hz, 1H), 7.82 (ddd, J=8.2, 1.7, 0.6 Hz, 1H), 7.68 (d, J=1.6 Hz, 1H), 7.53 (s, 1H), 7.49 (dd, J=7.7, 1.8 Hz, 1H), 7.30 (d, J=7.7 Hz, 1H), 4.45 (dd, J=5.6, 4.4 Hz, 2H), 4.13-4.03 (m, 2H), 3.81 (ddd, J=13.4, 10.0, 3.9 Hz, 2H), 3.61 (tt, J=15.2, 4.0 Hz, 1H), 3.51 (dd, J=5.4, 4.2 Hz, 2H), 3.36-3.34 (m, 4H), 3.15-2.83 (m, 4H), 2.45 (t, J=10.1 Hz, 2H), 1.64 (p, J=12.0 Hz, 2H). ESI MS [M+H]⁺ for C₃₀H₃₂N₅O₃, calcd. 510.2, found 510.2.

### Example 48: 8-{5-[7-(Piperazin-1-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

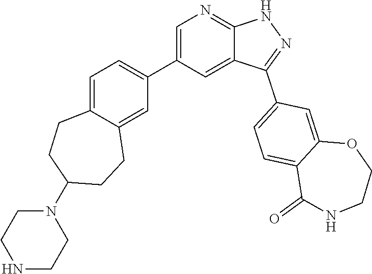 [SMILES: O=C1NCCOc2cc(-c3n[nH]c4ncc(-c5ccc6c(c5)CCC(N5CCNCC5)CC6)cc34)ccc21]

The title compound was prepared in a similar manner to Example 42. ¹H NMR (400 MHz, Methanol-d₄) δ 8.78 (d, J=2.0 Hz, 1H), 8.57 (d, J=2.0 Hz, 1H), 8.00 (d, J=8.2 Hz, 1H), 7.82 (dd, J=8.2, 1.7 Hz, 1H), 7.68 (d, J=1.7 Hz, 1H), 7.55 (d, J=2.0 Hz, 1H), 7.50 (dd, J=7.7, 2.0 Hz, 1H), 7.31 (d, J=7.8 Hz, 1H), 4.46 (dd, J=5.3, 4.2 Hz, 2H), 3.73 (tt, J=11.9, 2.5 Hz, 1H), 3.60 (s, 8H), 3.51 (t, J=5.3, 4.4 Hz, 2H), 3.16-2.86 (m, 4H), 2.44 (t, J=10.0 Hz, 2H), 1.66 (p, J=12.3 Hz, 2H). ESI MS [M+H]⁺ for C₃₀H₃₃N₆O₂, calcd. 509.3, found 509.2.

### Example 49: 8-(5-{7-[(2R)-2-(Hydroxymethyl)pyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

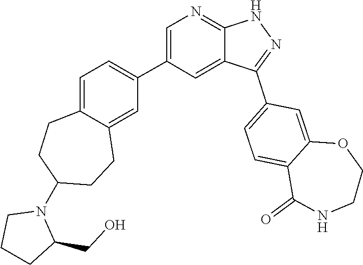 [SMILES: O=C1NCCOc2cc(-c3n[nH]c4ncc(-c5ccc6c(c5)CCC(N5CCC[C@@H]5CO)CC6)cc34)ccc21]

The title compound was prepared in a similar manner to Example 42. ¹H NMR (400 MHz, DMSO-d₆) δ 8.83 (d, J=2.0 Hz, 1H), 8.63 (d, J=2.1 Hz, 1H), 8.36 (t, J=5.4 Hz, 1H), 7.92 (d, J=8.2 Hz, 1H), 7.86 (dd, J=8.2, 1.6 Hz, 1H), 7.65 (dd, J=1.7, 0.5 Hz, 1H), 7.58 (d, J=2.0 Hz, 1H), 7.52 (dd, J=7.6, 2.0 Hz, 1H), 7.23 (d, J=7.7 Hz, 1H), 4.34 (dd, J=5.4, 4.1 Hz, 2H), 3.40-3.32 (m, 3H), 3.08 (t, J=9.0 Hz, 1H), 2.98-2.59 (m, 7H), 2.03 (m, 2H), 1.69-1.18 (m, 8H). ESI MS [M+H]⁺ for C₃₁H₃₄N₅O₃, calcd. 524.3, found 524.2.

### Example 50: 8-(5-{7-[(2S)-2-(Hydroxymethyl)pyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

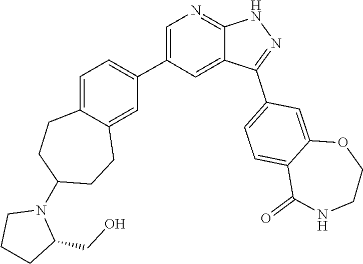 [SMILES: O=C1NCCOc2cc(-c3n[nH]c4ncc(-c5ccc6c(c5)CCC(N5CCC[C@H]5CO)CC6)cc34)ccc21]

The title compound was prepared in a similar manner to Example 42. ¹H NMR (400 MHz, DMSO-d₆) δ 8.83 (d, J=2.1 Hz, 1H), 8.63 (d, J=2.1 Hz, 1H), 8.37 (t, J=5.4 Hz, 1H), 7.92 (d, J=8.2 Hz, 1H), 7.86 (dd, J=8.2, 1.7 Hz, 1H), 7.65 (d, J=1.6 Hz, 1H), 7.58 (d, J=2.0 Hz, 1H), 7.51 (dd, J=7.7, 2.0 Hz, 1H), 7.23 (d, J=7.8 Hz, 1H), 4.34 (dd, J=5.4, 4.1 Hz, 2H), 3.40-3.34 (m, 4H), 3.10 (dd, J=10.5, 7.7 Hz, 1H), 2.97-2.58 (m, 8H), 2.03 (s, 2H), 1.68-1.14 (m, 7H). ESI MS [M+H]⁺ for C₃₁H₃₄N₅O₃, calcd. 524.3, found 524.2.

### Example 51: 8-(5-(7-((R)-3-Hydroxypyrrolidin-1-yl)-6,7,8,9-tetrahydro-5H-benzo[7]annulen-2-yl)-1H-pyrazolo[3,4-b]pyridin-3-yl)-3,4-dihydrobenzo[f][1,4]oxazepin-5(2H)-one

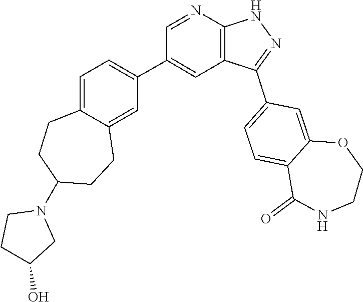 [SMILES: O=C1NCCOc2cc(-c3n[nH]c4ncc(-c5ccc6c(c5)CCC(N5CC[C@@H](O)C5)CC6)cc34)ccc21]

The title compound was prepared in a similar manner to Example 42. ¹H NMR (400 MHz, DMSO-d₆) δ 8.83 (d, J=2.1 Hz, 1H), 8.63 (d, J=2.1 Hz, 1H), 8.37 (t, J=5.4 Hz, 1H), 7.95-7.82 (m, 2H), 7.65 (dd, J=1.7, 0.5 Hz, 1H), 7.58 (s, 1H), 7.51 (d, J=7.8 Hz, 1H), 7.23 (d, J=7.8 Hz, 1H), 4.34 (dd, J=5.4, 4.1 Hz, 2H), 4.19 (s, 1H), 3.35 (d, J=4.6 Hz, 2H), 3.02 (s, 4H), 2.86 (s, 1H), 2.69 (s, 4H), 1.96 (d, J=13.4 Hz, 2H), 1.53 (s, 4H). ESI MS [M+H]⁺ for C₃₀H₃₂N₅O₃, calcd. 510.2, found 510.2.

### Example 52: 8-(5-(7-(Azetidin-1-yl)-6,7,8,9-tetrahydro-5H-benzo[7]annulen-2-yl)-1H-pyrazolo[3,4-b]pyridin-3-yl)-3,4-dihydrobenzo[f][1,4]oxazepin-5(2H)-one

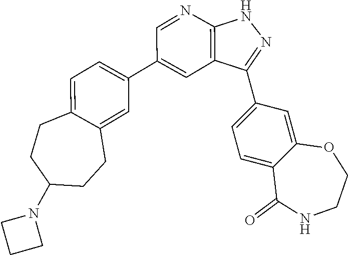

The title compound was prepared in a similar manner to Example 42. ¹H NMR (400 MHz, DMSO-d₆) δ 9.84 (s, 1H), 8.85 (d, J=2.1 Hz, 1H), 8.65 (d, J=2.1 Hz, 1H), 8.37 (t, J=5.4 Hz, 1H), 7.96-7.82 (m, 2H), 7.67-7.55 (m, 3H), 7.28 (d, J=7.8 Hz, 1H), 4.34 (dd, J=5.4, 4.1 Hz, 2H), 4.17 (p, J=9.5 Hz, 2H), 4.01 (d, J=9.6 Hz, 2H), 3.46-3.31 (m, 4H), 2.97 (dd, J=14.5, 7.4 Hz, 1H), 2.88 (dd, J=14.6, 7.3 Hz, 1H), 2.74 (q, J=11.5 Hz, 2H), 2.16 (s, 2H), 1.11 (p, J=12.4 Hz, 2H). ESI MS [M+H]⁺ for C₂₉H₃₀N₅O₂, calcd. 480.2, found 480.2.

### Example 53: 8-(5-(2,3,4,5-Tetrahydro-1H-benzo[d]azepin-7-yl)-1H-pyrazolo[3,4-b]pyridin-3-yl)-3,4-dihydrobenzo[f][1,4]oxazepin-5(2H)-one

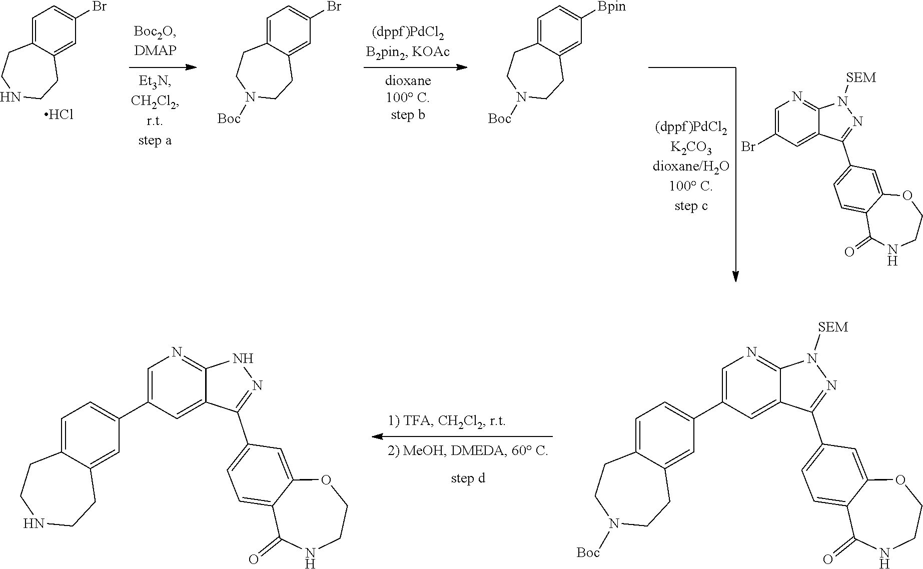

Step a: To a mixture of 7-bromo-2,3,4,5-tetrahydro-1H-3-benzazepine hydrochloride (200 mg, 0.76 mmol), Et₃N (0.3 mL, 0.22 mmol), DMAP (10 mg, 0.8 mmol), and CH₂Cl₂ (5 mL) was added Boc₂O (176 mg, 0.76 mmol) and stirred at rt for 14 h. The reaction mixture was filtered to remove any insoluble material, concentrated and purified by column chromatography (SiO₂, 0 to 90% EtOAc in hexanes) to afford the desired product as a white solid (219 mg; 88%).

Step b: The desired product was prepared in a similar manner to Example 20, step b.

Step c: The desired product was prepared in a similar manner to Example 20, step c (351 mg; quantitative).

Step d: The desired product was prepared in a similar manner to Example 20, step d (45 mg; 59%). ¹H NMR (400 MHz, DMSO-d₆) δ 8.86 (d, J=2.1 Hz, 2H), 8.65 (d, J=2.1 Hz, 1H), 8.38 (t, J=5.4 Hz, 1H), 7.93 (dd, J=8.2, 0.4 Hz, 1H), 7.85 (dd, J=8.2, 1.7 Hz, 1H), 7.74-7.61 (m, 3H), 7.34 (d, J=7.8 Hz, 1H), 4.34 (dd, J=5.4, 4.1 Hz, 2H), 3.36 (q, J=5.0 Hz, 2H), 3.29-3.05 (m, 8H). ESI MS [M+H]⁺ for C₂₅H₂₄N₅O₂, calcd. 426.2, found 426.2.

### Example 54: 8-(5-(1,2,3,4-Tetrahydroisoquinolin-6-yl)-1H-pyrazolo[3,4-b]pyridin-3-yl)-3,4-dihydrobenzo[f][1,4]oxazepin-5(2H)-one

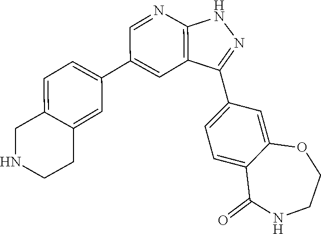

The title compound was prepared in a similar manner to Example 53. ¹H NMR (400 MHz, DMSO-d₆) δ 9.04 (s, 1H), 8.87 (d, J=2.1 Hz, 1H), 8.69 (d, J=2.1 Hz, 1H), 8.38 (t, J=5.4 Hz, 1H), 7.93 (dd, J=8.2, 0.4 Hz, 1H), 7.86 (dd, J=8.2, 1.7 Hz, 1H), 7.73 (d, J=7.1 Hz, 2H), 7.65 (dd, J=1.6, 0.4 Hz, 1H), 7.34 (d, J=8.4 Hz, 1H), 4.43-4.27 (m, 4H), 3.40-3.29 (m, 4H), 3.08 (t, J=6.3 Hz, 2H). ESI MS [M+H]⁺ for C₂₄H₂₂N₅O₂, calcd. 412.2, found 412.2.

### Example 55: 8-(5-(1,2,3,4-Tetrahydroisoquinolin-7-yl)-1H-pyrazolo[3,4-b]pyridin-3-yl)-3,4-dihydrobenzo[f][1,4]oxazepin-5(2H)-one

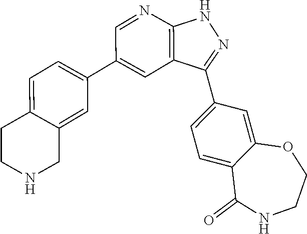

The title compound was prepared in a similar manner to Example 53. ¹H NMR (400 MHz, DMSO-d₆) δ 9.06 (s, 1H), 8.87 (d, J=2.1 Hz, 1H), 8.67 (d, J=2.1 Hz, 1H), 8.38 (t, J=5.4 Hz, 1H), 7.93 (dd, J=8.2, 0.4 Hz, 1H), 7.85 (dd, J=8.2, 1.7 Hz, 1H), 7.80-7.70 (m, 2H), 7.65 (dd, J=1.7, 0.4 Hz, 1H), 7.36 (d, J=8.0 Hz, 1H), 4.38-4.31 (m, 4H), 3.37 (tt, J=13.6, 7.5 Hz, 4H), 3.03 (t, J=6.2 Hz, 2H). ESI MS [M+H]⁺ for C₂₄H₂₂N₅O₂, calcd. 412.2, found 412.2.

### Example 56: 8-{5-[(7R)-7-(Pyrrolidin-1-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one Example 57: 8-{5-[(7S)-7-(Pyrrolidin-1-yl)-6,7,8,9-tetrahydro-5H-benzo[7]annulen-2-yl]-1H-pyrazolo[3,4-b]pyridin-3-yl}-2,3,4,5-tetrahydro-1,4-benzoxazepin-5-one

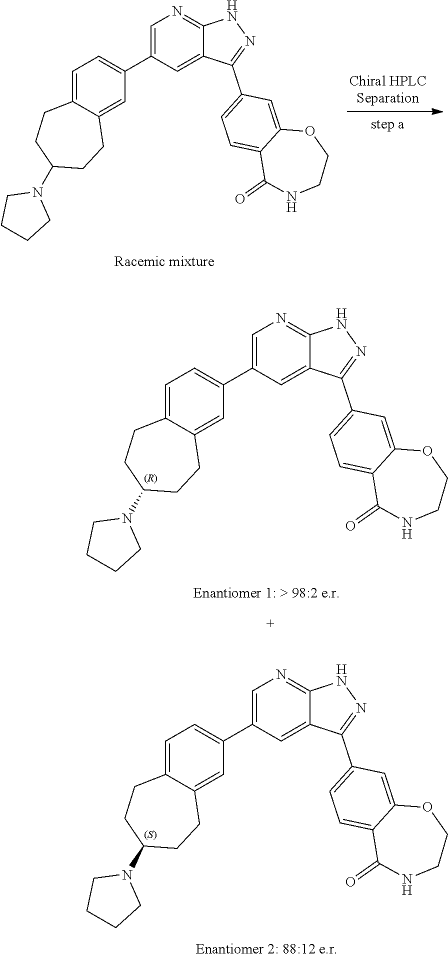

Step a: The racemic material from Example 1 was separated using a semi-prep chiral AD-H column (20×250 mm; 30% EtOH in hexanes+0.1% Et₂NH). Enantiomer 1 (analytical retention time=18.7 min): white powder, 8 mg, >98:2 e.r. and was arbitrarily assigned as Example 56. Enantiomer 2 (analytical retention time=24.5 min): white powder, 12 mg, 88:12 e.r. and was subsequently assigned as Example 57.

### Example 58: 8-(5-{3-Fluoro-7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

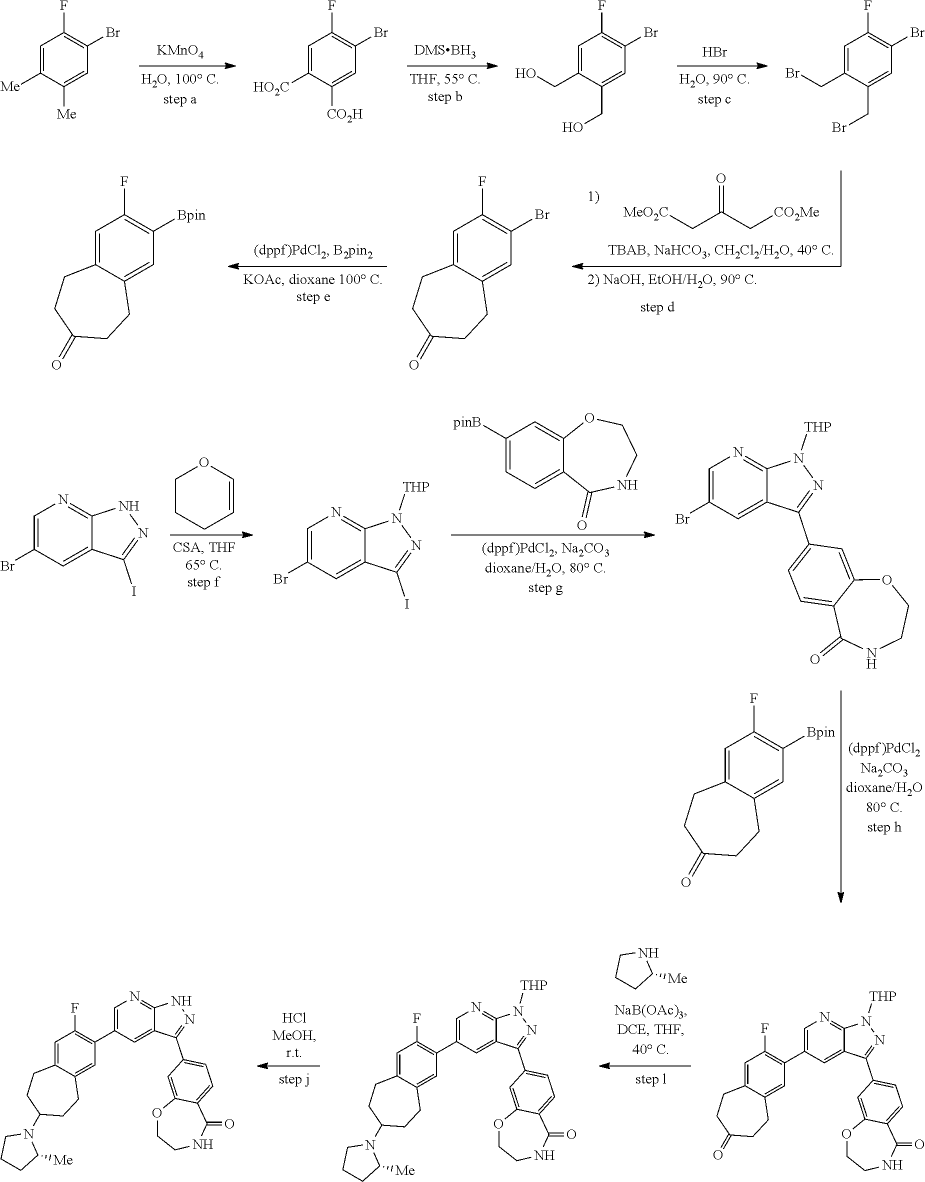

Step a: A mixture of 1-bromo-2-fluoro-4,5-dimethylbenzene (1.03 g, 5.07 mmol), KMnO₄ (3.21 g, 20.3 mmol), and water (41 mL) was stirred at 100° C. for 14 hours, quenched with 10% wt. NaHSO_{3(aq)} (20 mL), and adjusted to pH-12 with 2M NaOH_{(aq)}. The solids were removed by filtration and washed with water. The filtrate was acidified to pH-2 with 4M HCl(aq), extracted with 4:1 CH₂Cl₂/IPA (1×250 mL), dried over Na₂SO₄, and concentrated to afford the desired product as a white solid (418 mg; 31%).

Step b: To a mixture of the product from step a (418 g, 1.59 mmol) and THE (7.9 mL) at 0° C. was added borane dimethylsulfide (452 μL, 4.77 mmol) dropwise. The reaction mixture was stirred at 0° C. for 10 minutes, then warmed to and stirred at 55° C. for 14 hours. The mixture was cooled to r.t., 2M NaOH_{(aq)} (7.2 mL) was added dropwise, and the mixture stirred at r.t. for 1 hour. 12M HCl_{(aq)} (0.10 mL) was added dropwise and the resultant organic phase was concentrated and diluted with EtOAc (10 mL). The resultant aqueous phase was extracted with EtOAc (1×10 mL), the combined organic phases were washed with 1:5 water:brine (10 mL), dried over Na₂SO₄, and concentrated to afford the desired product which was used crude in the next step.

Step c: A mixture of the product from step b and HBr (1.6 mL, 48% wt. in H₂O) was stirred at 90° C. for 2 hours. The mixture was cooled to r.t., extracted with CH₂Cl₂ (3×10 mL), dried over Na₂SO₄, and concentrated to afford the desired product as a brown oil (534 mg; 93%; two steps).

Step d: A mixture of the product from step c (534 mg, 1.48 mmol), dimethyl 1,3-acetonedicarboxylate (309 mg, 1.78 mmol), tetrabutylammonium bromide (239 mg, 0.740 mmol), NaHCO₃ (622 mg, 7.40 mmol), CH₂Cl₂ (3.0 mL), and water (7.4 mL) was vigorously stirred at 40° C. for 3 days. The organic phase was separated, concentrated, diluted with EtOAc (10 mL), washed with 9:1 water:brine (4×10 mL), dried over Na₂SO₄, and concentrated. The residue was dissolved in EtOH (11 mL) and 2M NaOH_{(aq)} (7.4 mL) was added. The reaction mixture was stirred at 90° C. for 2 hours. The mixture was cooled to r.t. and adjusted to pH-7 by addition of 12M HCl_{(aq)}. The EtOH was removed under reduced pressure and the resultant aqueous phase was extracted with CH₂Cl₂ (3×10 mL). The organic phase was dried over Na₂SO₄ and concentrated. The crude material was purified by column chromatography (24 g silica gel, hexanes:EtOAc) 0% to 20% gradient (20 minutes); 20% to 35% gradient (10 minutes) to afford the desired product as an orange oil (141 mg; 37%).

Step e: The desired product was prepared in a similar manner to Example 1, step d.

Step f: The desired product was prepared in a similar manner to Example 1, step g (26.8 g; 85%).

Step g: The desired product was prepared in a similar manner to Example 7, step c (341 mg; 77%).

Step h: The desired product was prepared in a similar manner to Example 7, step c (199 mg; 67%).

Step i: To a mixture of the product from step h (199 mg, 0.368 mmol), (R)-2-methylpyrrolidine (38 mg, 0.44 mmol), AcOH (21 μL, 0.37 mmol), and DCE (1.8 mL) at r.t. was added NaBH(OAc)₃ (117 mg, 0.552 mmol). The reaction mixture was stirred at 40° C. for 14 hours, quenched with sat. NaHCO_{3(aq)} (10 mL) and extracted with CH₂Cl₂ (10 mL). The organic phase was dried over Na₂SO₄ and concentrated to afford the desired product which was used crude in the next step.

Step j: A mixture of the product from step i (0.368 mmol assumed) and 3M HCl in MeOH (3.7 mL) was stirred at r.t. for 5 hours and diluted with MTBE (30 mL) The precipitated solids were collected by filtration and washed with MTBE. The crude material was purified by column chromatography (43 g C18, (H₂O/ACN)+0.1% TFA) 5% to 50% gradient (25 minutes) to afford the desired product as a white solid (50 mg; 26%). ¹H NMR (400 MHz, DMSO-d₆) δ 8.72 (t, J=2.0 Hz, 1H), 8.64 (d, J=1.2 Hz, 1H), 8.40 (t, J=5.4 Hz, 1H), 7.94 (d, J=8.2 Hz, 1H), 7.85 (dd, J=8.2, 1.7 Hz, 1H), 7.65 (d, J=1.4 Hz, 1H), 7.48 (dd, J=8.2, 2.4 Hz, 1H), 7.17 (dd, J=11.4, 2.5 Hz, 1H), 4.37 (dd, J=5.4, 4.1 Hz, 2H), 3.38 (q, J=5.1 Hz, 2H), 2.99-2.78 (m, 4H), 2.78-2.62 (m, 3H), 2.44 (q, J=8.3 Hz, 1H), 2.05-1.91 (m, 2H), 1.87-1.75 (m, 1H), 1.68-1.51 (m, 2H), 1.45 (q, J=12.1 Hz, 1H), 1.34-1.21 (m, 2H), 1.02 (d, J=6.0 Hz, 3H). ESI MS [M+H]⁺ for C₃₁H₃₃FN₅O₂, calcd. 526.3, found 526.2.

### Example 59: (2R)-2-Methyl-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

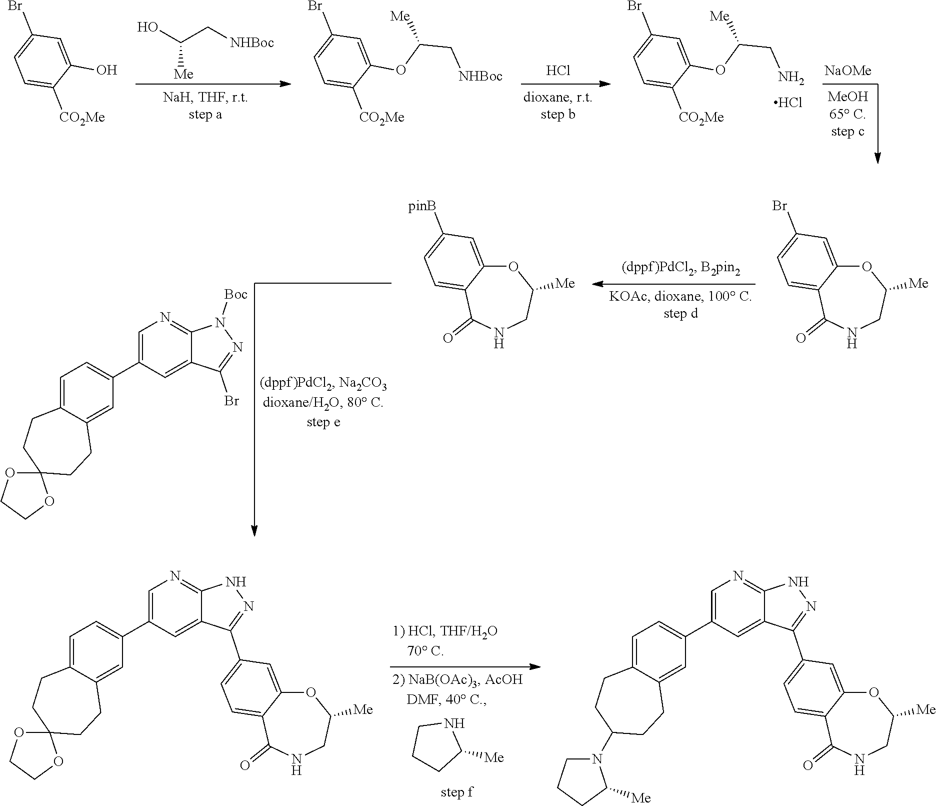

Step a: To a mixture of methyl 4-bromo-2-hydroxybenzoate (2.31 g, 10.0 mmol), tert-butyl ((S)-2-hydroxypropyl)carbamate (1.75 g, 10.0 mmol), PPh₃ (2.75 g, 10.5 mmol), and THF (25 mL) at 0° C. was added diisopropyl azodicarboxylate (2.07 mL, 10.5 mmol) dropwise. The reaction mixture was stirred at 0° C. for 30 minutes, stirred at r.t. for 14 hours, concentrated onto silica gel, and purified by column chromatography (80 g silica gel, hexanes:EtOAc) 0% to 35% gradient (25 minutes) to afford the desired product as a colorless oil (3.44 g; 89%).

Step b: The desired product was prepared in a similar manner to Example 1, step b (2.79 g; 97%).

Step c: The desired product was prepared in a similar manner to Example 1, step c (2.08 g; 94%).

Step d: The desired product was prepared in a similar manner to Example 1, step d.

Step e: The desired product was prepared in a similar manner to Example 7, step c (113 mg; 57%).

Step f: A mixture of the product from step e (113 mg, 0.228 mmol), HCl (455 μL, 0.455 mmol, 1M in water), and THE (1.1 mL) was stirred at 70° C. for 1 hour, cooled to r.t., neutralized with sat. NaHCO_{3(aq)} (1.0 mL), diluted with water (20 mL) and filtered to collect the precipitated solids. The solids were washed with water and dried. To a mixture of this solid, (R)-2-methylpyrrolidine (31 mg, 0.37 mmol), AcOH (21 μL, 0.37 mmol), and DMF (0.90 mL) at r.t. was added NaBH(OAc)₃ (97 mg, 0.46 mmol). The reaction mixture was stirred at 40° C. for 3 hours, diluted with EtOAc (18 mL), water (18 mL), and brine (3.0 mL). The aqueous phase was adjusted to pH-12 with 2M NaOH_{(aq)}. The organic phase was washed with water:2M NaOH_{(aq)}:brine (8:1:1) (1×10 mL), dried over Na₂SO₄, and concentrated. The crude material was purified by column chromatography (43 g C18, (H₂O/ACN)+0.1% TFA) 5% to 50% gradient (25 minutes) to afford the desired product as an off-white solid (87 mg; 77%). ¹H NMR (400 MHz, DMSO-d₆) δ 8.87 (d, J=2.1 Hz, 1H), 8.66 (d, J=1.8 Hz, 1H), 8.40 (t, J=5.7 Hz, 1H), 7.95 (dd, J=8.1, 1.7 Hz, 1H), 7.79 (d, J=8.4 Hz, 1H), 7.65 (d, J=1.6 Hz, 1H), 7.61 (t, J=2.3 Hz, 1H), 7.55 (dd, J=7.7, 1.9 Hz, 1H), 7.27 (dd, J=7.9, 1.3 Hz, 1H), 4.59 (td, J=6.4, 3.6 Hz, 1H), 3.33-3.29 (m, 1H), 3.10-3.00 (m, 1H), 3.00-2.76 (m, 5H), 2.76-2.65 (m, 2H), 2.44 (q, J=8.2 Hz, 1H), 2.06-1.94 (m, 2H), 1.87-1.76 (m, 1H), 1.69-1.49 (m, 2H), 1.49-1.39 (m, 1H), 1.35-1.22 (m, 5H), 1.02 (d, J=6.0 Hz, 3H). ESI MS [M+H]⁺ for C₃₂H₃₆N₅O₂, calcd. 522.3, found 522.3.

### Example 60: 9-Fluoro-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

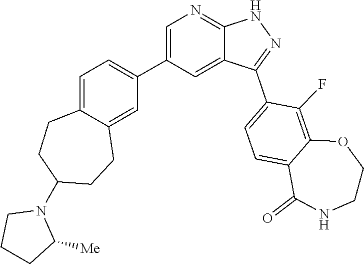 [SMILES: C[C@@H]1CCCN1C1CCc2ccc(-c3cnc4[nH]nc(-c5ccc6c(c5F)OCCNC6=O)c4c3)cc2CC1]

The title compound was prepared in a similar manner to Example 59. ¹H NMR (400 MHz, DMSO-d₆) δ 8.89 (d, J=2.0 Hz, 1H), 8.54 (t, J=5.4 Hz, 1H), 8.41 (s, 1H), 7.71 (dd, J=8.4, 1.3 Hz, 1H), 7.63 (dd, J=8.4, 6.2 Hz, 1H), 7.55 (s, 1H), 7.49 (dd, J=7.8, 2.0 Hz, 1H), 7.26 (d, J=7.8 Hz, 1H), 4.53-4.41 (m, 2H), 3.44 (q, J=5.1 Hz, 2H), 2.99-2.75 (m, 5H), 2.74-2.63 (m, 2H), 2.47-2.40 (m, 1H), 2.34-2.30 (m, 1H), 2.05-1.94 (m, 2H), 1.87-1.74 (m, 1H), 1.68-1.50 (m, 2H), 1.48-1.38 (m, 1H), 1.34-1.21 (m, 3H), 1.02 (d, J=5.9 Hz, 3H). ESI MS [M+H]⁺ for C₃₁H₃₃FN₅O₂, calcd. 526.3, found 526.2.

### Example 61: 7-Fluoro-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

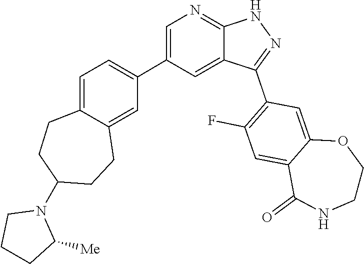 [SMILES: C[C@@H]1CCCN1C1CCc2ccc(-c3cnc4[nH]nc(-c5cc6c(cc5F)C(=O)NCCO6)c4c3)cc2CC1]

The title compound was prepared in a similar manner to Example 59. ¹H NMR (400 MHz, DMSO-d₆) δ 8.88 (d, J=2.1 Hz, 1H), 8.56 (d, J=5.4 Hz, 1H), 8.41 (t, J=2.6 Hz, 1H), 7.71 (d, J=11.2 Hz, 1H), 7.56-7.50 (m, 2H), 7.50-7.46 (m, 1H), 7.25 (d, J=7.7 Hz, 1H), 4.39-4.30 (m, 2H), 3.42-3.37 (m, 2H), 2.97-2.75 (m, 4H), 2.75-2.65 (m, 2H), 2.47-2.38 (m, 1H), 2.34-2.30 (m, 1H), 2.04-1.93 (m, 2H), 1.86-1.76 (m, 2H), 1.65-1.52 (m, 1H), 1.49-1.37 (m, 1H), 1.35-1.20 (m, 1H), 1.02 (d, J=6.0 Hz, 3H). ESI MS [M+H]⁺ for C₃₁H₃₃FN₅O₂, calcd. 526.3, found 526.2.

### Example 62: (2S)-2-Methyl-8-(5-{7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

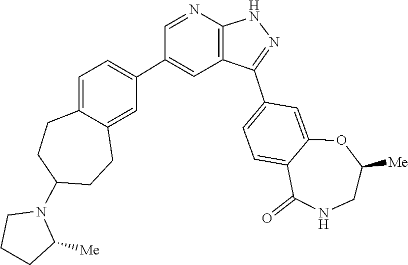

The title compound was prepared in a similar manner to Example 59. ¹H NMR (400 MHz, DMSO-d₆) δ 8.87 (d, J=2.1 Hz, 1H), 8.66 (d, J=2.1 Hz, 1H), 8.40 (t, J=5.7 Hz, 1H), 7.95 (ddd, J=8.1, 1.7, 0.8 Hz, 1H), 7.79 (dd, J=8.2, 0.6 Hz, 1H), 7.65 (d, J=1.3 Hz, 1H), 7.64-7.58 (m, 1H), 7.55 (dd, J=7.9, 1.8 Hz, 1H), 7.27 (d, J=6.7 Hz, 1H), 4.59 (td, J=6.4, 3.6 Hz, 1H), 3.33-3.28 (m, 1H), 3.05 (dt, J=15.3, 6.0 Hz, 1H), 3.00-2.76 (m, 5H), 2.76-2.67 (m, 2H), 2.44 (q, J=8.2 Hz, 1H), 2.07-1.95 (m, 2H), 1.87-1.76 (m, 1H), 1.69-1.50 (m, 2H), 1.49-1.39 (m, 1H), 1.34-1.22 (m, 5H), 1.02 (d, J=6.0 Hz, 3H). ESI MS [M+H]⁺ for C₃₂H₃₆N₅O₂, calcd. 522.3, found 522.3.

### Example 63: 8-{5-[7-(Pyrrolidin-1-yl)-5H,6H,7H,8H,9H-cyclohepta[b]pyridin-2-yl]-1H-pyrazolo[3,4-b]pyridin-3-yl}-2,3,4,5-tetrahydro-1,4-benzoxazepin-5-one

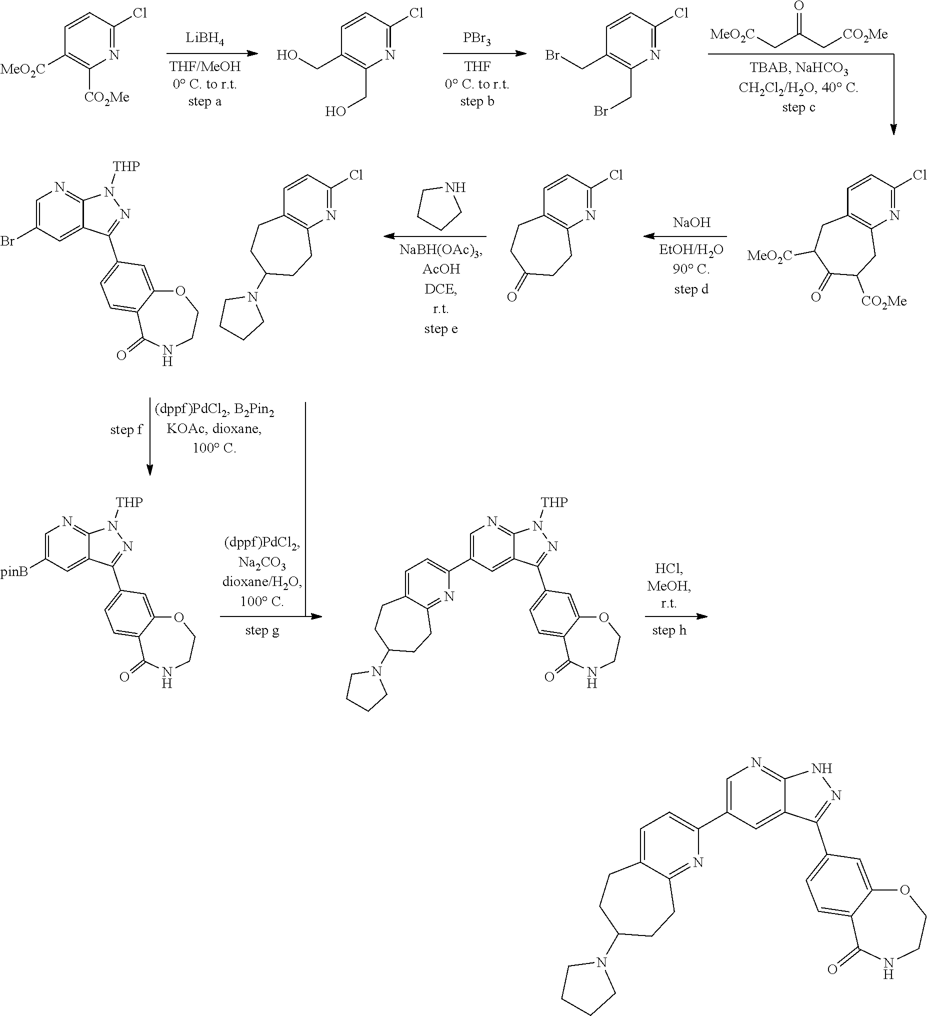

Step a: Lithium borohydride (2.0 M solution in THF) (13.6 mL, 27.2 mmol) was added dropwise to a solution of dimethyl 6-chloropyridine-2,3-dicarboxylate (2.50 g, 10.9 mmol) in 38:1 THF:MeOH (34.5 mL) at 0° C. The cold bath was removed and the mixture was stirred at rt for 2.5 h. The mixture was poured in to sat. NaHCO_{3(aq)} (100 mL) and product was extracted into EtOAc (5×100 mL). The combined organic phase was dried (Na₂SO₄), concentrated and taken crude into the next step.

Step b: Phosphorus tribromide (1.33 mL, 9.28 mmol) was added dropwise to a suspension of the crude product of step a (10.9 mmol) in THE (54 mL) at 0° C. The cold bath was removed and the mixture was stirred at rt for 5 h. The mixture was then cooled to 0° C. and neutralized cautiously with NaHCO_{3(aq)} (150 mL). The layers were separated and additional product was extracted into CH₂Cl₂ (2×150 mL). The combined organic phase was dried (Na₂SO₄), concentrated and taken crude into the next step.

Step c: A mixture of the crude product from step b (10.9 mmol), 1,5-dimethyl 3-oxopentanedioate (1.42 mL, 9.82 mmol), TBAB (1.32 g, 4.09 mmol), sodium bicarbonate (3.44 g, 40.9 mmol), CH₂Cl₂ (16.4 mL) and H₂O (40.9 mL) was heated at 40° C. overnight. The CH₂Cl₂was removed in vacuo and the residue was dissolved in EtOAc (40 mL). The solution was washed with 9:1 H₂O:brine (4×40 mL), dried (Na₂SO₄), concentrated and taken crude into the next step.

Step d: The crude product from step c was dissolved in EtOH (63 mL) and in 2 N NaOH_{(aq)} (42 mL) was added. The mixture was heated at 90° C. for 2 h. The EtOH was removed in vacuo and the solution was acidified to pH 6 with 12 N HCl_{(aq)}. Product was extracted into CH₂Cl₂ (2×30 mL) and the combined was dried (Na₂SO₄) and concentrated. The crude material was purified by flash chromatography (0 to 100% EtOAc in hexanes) to furnish the required product as a white solid (355 mg; 22%).

Step e: Sodium triacetoxyborohydride (288 mg, 1.36 mmol) and acetic acid (0.05 mL, 0.906 mmol) were added to a solution of the product from step d (177 mg, 0.906 mmol) and pyrrolidine (0.09 mL, 1.09 mmol) in DCE (4.5 mL) and the mixture was stirred at rt overnight. The reaction was quenched with sat. NaHCO_{3(aq)} (10 mL) and product was extracted into CH₂Cl₂ (3×10 mL). The combined organic phase was washed with brine (10 mL), dried (Na₂SO₄), concentrated and taken crude into the next step.

Step f: The desired product was prepared in a similar manner to Example 1, step d.

Step g: The desired product was prepared in a similar manner to Example 26, step c (60.3 mg; 33%).

Step h: 3N HCl in MeOH (2.1 mL) was added to the product from step g (60.3 mg, 0.104 mmol) and the mixture was stirred at rt overnight. The reaction was concentrated and the crude product was triturated successively with MTBE and ACN to afford the desired product (26.6 mg; 42%). ¹H NMR (400 MHz, DMSO-d₆) δ 11.22 (br. s, 1H), 9.21 (d, J=24.7 Hz, 2H), 8.49-8.41 (m, 1H), 8.21 (d, J=7.5 Hz, 1H), 8.15 (br. s, 1H), 7.99 (d, J=8.1 Hz, 1H), 7.94 (d, J=7.4 Hz, 1H), 7.74 (d, J=1.4 Hz, 1H), 4.44-4.37 (m, 2H), 3.67-3.55 (m, 2H), 3.54-3.38 (m, 4H), 3.25-3.08 (m, 2H), 2.92 (t, J=13.3 Hz, 1H), 2.52-2.41 (m, 4H), 2.04-1.84 (m, 4H), 1.85-1.75 (m, 1H), 1.73-1.61 (m, 1H). ESI MS [M+H]⁺ for C₂₉H₃₁N₆O₂, calcd. 495.3, found 495.2.

### Example 64: 8-(5-{3-Methyl-7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

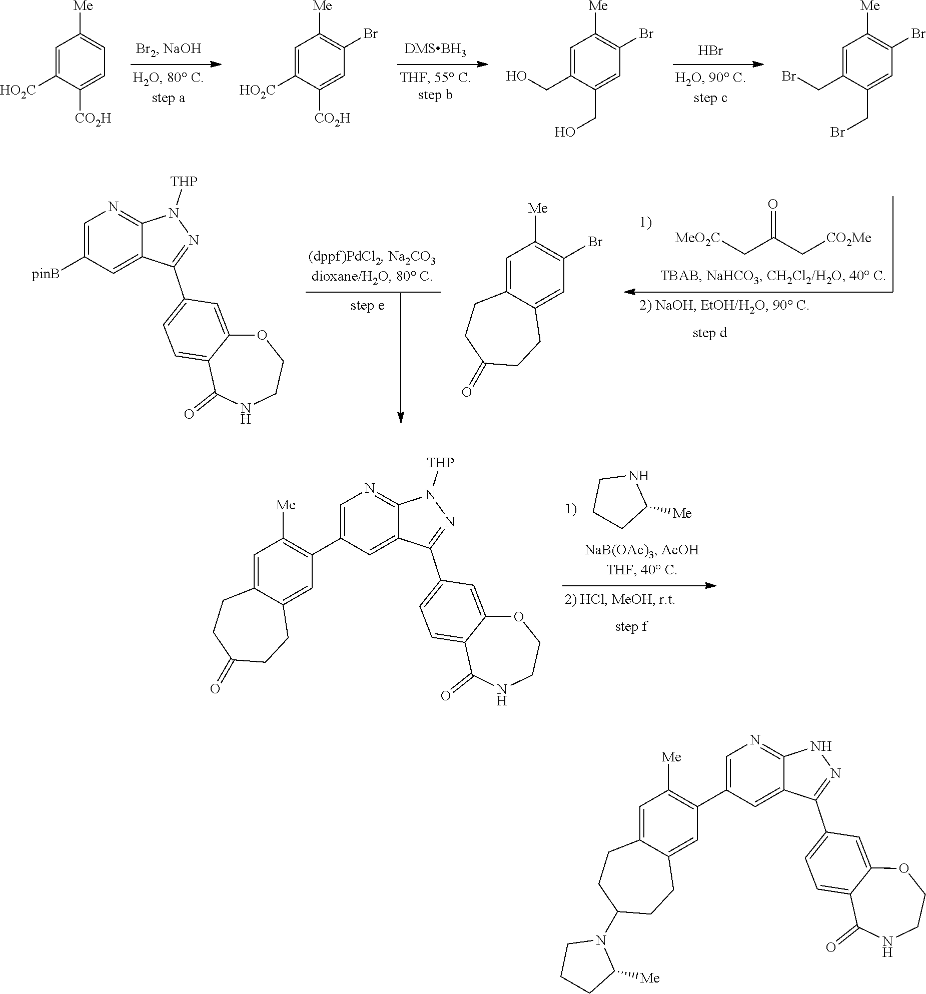

Step a: To a mixture of 4-methylphthalic acid (18.0 g, 100 mmol), NaOH (12.0 g, 300 mmol), and water (100 mL) at 0° C. was added Br₂ (5.12 mL, 100 mmol) dropwise. Upon completion, the reaction mixture was warmed to and stirred at 80° C. for 1.5 hours. The mixture was cooled to r.t. and water (100 mL) was added, followed by 2M HCl_{(aq)} (150 mL). The solids were collected by filtration, washed with water, and dried to afford the desired product as a white solid (5.58 g; 22%).

Step b: To a mixture of the product from step a (5.70 g, 22.0 mmol) and THE (110 mL) at 0° C. was added borane dimethylsulfide (6.26 mL, 66.0 mmol) dropwise. The reaction mixture was stirred at 0° C. for 10 minutes, then warmed to and stirred at 55° C. for 14 hours. The mixture was cooled to r.t., 2M NaOH_{(aq)} (100 mL) was added dropwise, and the mixture stirred at r.t. for 1 hour. 12M HCl_{(aq)} (17 mL) was added dropwise, the resultant organic phase concentrated, and diluted with EtOAc (50 mL). The resultant aqueous phase was extracted with EtOAc (1×50 mL), the combined organic phases were washed with 1:5 water:brine (60 mL), dried over Na₂SO₄, and concentrated to afford the desired product as a white solid (4.57 g; 90%).

Step c: A mixture of the product from step b and HBr (20 mL, 48% wt. in H₂O) was stirred at 90° C. for 2 hours. The mixture was cooled to r.t., the solids collected by filtration, and washed with water to afford the desired product which was used crude in the next step.

Step d: A mixture of the product from step c (19.8 mmol assumed), dimethyl 1,3-acetonedicarboxylate (4.14 g, 23.6 mmol), tetrabutylammonium bromide (3.19 g, 9.90 mmol), NaHCO₃ (8.32 g, 99.0 mmol), CH₂Cl₂ (40 mL), and water (99 mL) was vigorously stirred at 40° C. for 4 days. The organic phase was separated, concentrated, diluted with EtOAc (100 mL), washed with 9:1 water:brine (4×100 mL), dried over Na₂SO₄, and concentrated. The residue was dissolved in EtOH (152 mL) and 2M NaOH_{(aq)} (99 mL) was added. The reaction mixture was stirred at 90° C. for 2 hours. The mixture was cooled to r.t. and adjusted to pH-7 by addition of 12M HCl_{(aq)} (15 mL). The EtOH was removed under reduced pressure and the resultant aqueous phase was extracted with CH₂Cl₂ (150 L). The organic phase was dried over Na₂SO₄ and concentrated. The crude material was purified by column chromatography (80 g silica gel, hexanes:EtOAc) 0% to 20% gradient (20 minutes); 20% to 35% gradient (10 minutes) to afford the desired product as a light yellow solid (2.35 g; 47%; two steps).

Step e: The desired product was prepared in a similar manner to Example 7, step c (138 mg; 85%).

Step f: To a mixture of the product from step e (138 mg, 0.257 mmol), (R)-2-methylpyrrolidine (44 mg, 0.51 mmol), AcOH (30 μL, 0.51 mmol), and THE (1.3 mL) at r.t. was added NaBH(OAc)₃ (136 mg, 0.643 mmol). The reaction mixture was stirred at 40° C. for 3 hours, diluted with EtOAc (15 mL), water (15 mL), and brine (2.0 mL). The aqueous phase was adjusted to pH-12 with 2M NaOH_{(aq)}. The organic phase was washed with water:2M NaOH_{(aq)}:brine (8:1:1) (1×20 mL), dried over Na₂SO₄, and concentrated. 3M HCl in MeOH (1.3 mL) was added. The reaction mixture was stirred at r.t. for 2 hours and diluted with MTBE (20 mL). The precipitated solids were collected by filtration and washed with MTBE. The crude material was purified by column chromatography (43 g C18, (H₂O/ACN)+0.1% TFA) 5% to 50% gradient (25 minutes) to afford the desired product as a white solid (43 mg; 32%). ¹H NMR (400 MHz, DMSO-d₆) δ 8.54 (dd, J=2.0, 0.4 Hz, 1H), 8.47 (dd, J=2.0, 0.9 Hz, 1H), 8.39 (t, J=5.4 Hz, 1H), 7.91 (d, J=8.2 Hz, 1H), 7.85 (dd, J=8.2, 1.7 Hz, 1H), 7.65 (d, J=1.7 Hz, 1H), 7.12 (s, 1H), 7.11 (s, 1H), 4.35 (dd, J=5.4, 4.1 Hz, 2H), 3.37 (q, J=5.1 Hz, 2H), 2.95-2.59 (m, 7H), 2.43 (q, J=8.2 Hz, 1H), 2.20 (s, 3H), 2.06-1.90 (m, 2H), 1.86-1.76 (m, 1H), 1.68-1.49 (m, 2H), 1.43 (q, J=11.8 Hz, 1H), 1.34-1.20 (m, 2H), 1.02 (d, J=5.1 Hz, 3H). ESI MS [M+H]⁺ for C₃₂H₃₆N₅O₂, calcd. 522.3, found 522.3.

### Example 65: 8-(5-{4-Chloro-7-[(2R)-2-methylpyrrolidin-1-yl]-6,7,8,9-tetrahydro-5H-benzo[7]annulen-2-yl}-1H-pyrazolo[3,4-b]pyridin-3-yl)-2,3,4,5-tetrahydro-1,4-benzoxazepin-5-one

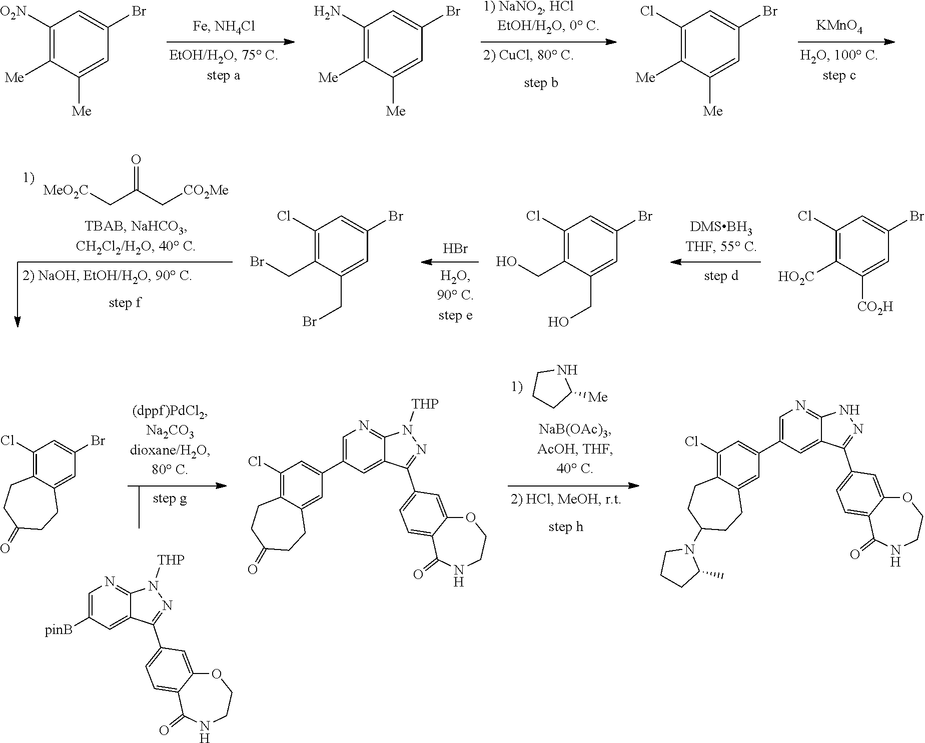

Step a: A mixture of 5-bromo-1,2-dimethyl-3-nitrobenzene (4.60 g, 20.0 mmol), iron powder (5.59 g, 100 mmol), NH₄Cl (5.35 g, 100 mmol), and 2:1 EtOH:water (80 mL) was stirred at 75° C. for 90 minutes, cooled to r.t., filtered through celite to remove solids (washing with EtOAc (200 mL)). The organic phase was dried over Na₂SO₄, concentrated, diluted with EtOAc (20 mL), again dried over Na₂SO₄, and again concentrated to afford the desired product as an orange oil (4.02 g; >100%).

Step b: To a mixture of the product from step a (4.02 g, 20.0 mmol) in EtOH (20 mL) at r.t. was added 12M HCl_{(aq)} (8.0 mL) dropwise. The mixture was cooled to 0° C. and a solution of NaNO₂ (1.79 g, 26.0 mmol) in water (8.0 mL) was added dropwise. The reaction mixture was stirred at 0° C. for 1 hour, cautiously charged with solid CuCl (3.96 g, 40.0 mmol) and 12M HCl_{(aq)} (8.0 mL), stirred at 80° C. for 1 hour, cooled to r.t., and extracted with hexanes (2×20 mL). The combined organic phases were dried over Na₂SO₄ and concentrated to afford the desired product as an orange oil (4.06 g; 92%; two steps).

Step c: The desired product was prepared in a similar manner to Example 58, step a (1.46 g; 28%).

Step d: The desired product was prepared in a similar manner to Example 58, step b (1.04 g; 80%).

Step e: The desired product was prepared in a similar manner to Example 64, step c (4.15 mmol; assume 100% yield).

Step f: The desired product was prepared in a similar manner to Example 64, step d (165 mg; 15%).

Step g: The desired product was prepared in a similar manner to Example 7, step c (235 mg; 70%).

Step h: The desired product was prepared in a similar manner to Example 64, step f (18 mg; 8%). ¹H NMR (400 MHz, DMSO-d₆) δ 8.89 (d, J=2.1 Hz, 1H), 8.74 (d, J=2.1 Hz, 1H), 8.40 (t, J=5.4 Hz, 1H), 7.96 (d, J=8.2 Hz, 1H), 7.91 (dd, J=8.3, 1.7 Hz, 1H), 7.78 (d, J=1.9 Hz, 1H), 7.70 (d, J=1.5 Hz, 1H), 7.66-7.62 (m, 1H), 4.38 (dd, J=5.4, 4.1 Hz, 2H), 3.43-3.35 (m, 2H), 3.08-2.90 (m, 2H), 2.90-2.78 (m, 2H), 2.77-2.67 (m, 2H), 2.67-2.56 (m, 1H), 2.48-2.39 (m, 1H), 2.08-1.95 (m, 2H), 1.88-1.76 (m, 1H), 1.67-1.50 (m, 2H), 1.50-1.35 (m, 1H), 1.36-1.15 (m, 2H), 1.02 (d, J=5.9 Hz, 3H). ESI MS [M+H]⁺ for C₃₁H₃₃C₁N₅O₂, calcd. 542.2, found 542.2.

### Example 66: 7-(5-{7-[(2R)-2-Methylpyrrolidin-1-yl]-6,7,8,9-tetrahydro-5H-benzo[7]annulen-2-yl}-1H-pyrazolo[3,4-b]pyridin-3-yl)-3,4-dihydro-2H-5,12,2-benzoxathiazepine-1,1-dione

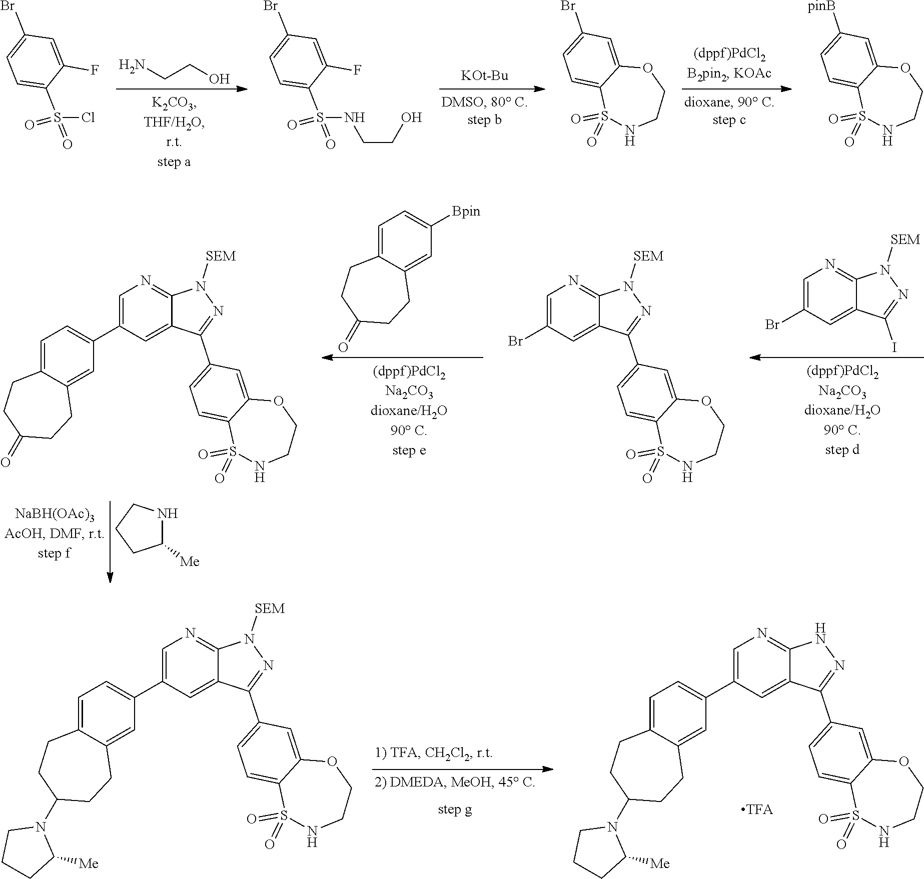

Step a: To a mixture of 4-bromo-2-fluorobenzenesulfonyl chloride (1.02 g, 3.73 mmol) in THE (8.2 mL) and H₂O (4.1 mL) was added K₂CO₃ (515 mg, 3.73 mmol) and the mixture stirred at r.t. for 10 min. 2-Aminoethanol was slowly added (0.22 mL, 3.73 mmol) and the reaction mixture stirred at r.t. for 16 h. EtOAc (15 mL) and H₂O (15 mL) were added and the layers separated. The aqueous layer was extracted with EtOAc (2×15 mL), then the combined organic layers were washed with brine, dried over MgSO₄, and concentrated to afford the product as a light brown solid (986 mg; 89%).

Step b: To a solution of the product of step a (986 mg, 3.31 mmol) in DMSO (6.6 mL) was added KOt-Bu (928 mg, 8.27 mmol) at r.t. and the reaction mixture was stirred at 80° C. for 24 h. Upon cooling, H₂O (10 mL) was added followed by sat. aq. NH₄Cl (10 mL) and the mixture extracted with EtOAc (3×15 mL). The combined organic layers were washed with brine, dried over anhyd. MgSO₄, concentrated, and purified by silica gel chromatography (100% CH₂Cl₂ to 10% EtOAc in CH₂Cl₂ to 10% MeOH in CH₂Cl₂) to afford the desired product as a white solid (544 mg; 59%).

Step c: To a mixture of the product of step b (245 mg, 0.881 mmol), B₂pin₂ (268 mg, 1.06 mmol), and KOAc (112 mg, 1.15 mmol) was added dioxane (8.8 mL), then the suspension was degassed with N₂ for 10 min. (dppf)PdCl₂ (32 mg, 0.0441 mmol) was added and the reaction mixture was stirred at 90° C. for 2.5 h. Upon cooling, EtOAc (20 mL) was added and the mixture was filtered through celite. The filtrate was concentrated to afford the crude material as a viscous brown oil.

Step d: To a mixture of the product of Example 1, step e (364 mg, 0.800 mmol), the crude product of step c (0.881 mmol), and Na₂CO₃ (170 mg, 1.60 mmol) was added dioxane (7.2 mL) and H₂O (0.80 mL), then the suspension was degassed with N₂ for 10 min. (dppf)PdCl₂ (29 mg, 0.0400 mmol) was added and the reaction mixture was stirred at 90° C. for 13 h. Upon cooling, CH₂Cl₂ (20 mL) was added and the mixture was dried over anhyd. MgSO₄, filtered, and concentrated. The residue was purified by silica gel chromatography (100% CH₂Cl₂ to 10% CH₂Cl₂ in MeOH) to afford the desired product as an orange solid (378 mg; 90%).

Step e: To a mixture of the product of step d (378 mg, 0.719 mmol), the product of Example 32, step h (1.34 mmol), and Na₂CO₃ (152 mg, 1.44 mmol) was added dioxane (6.5 mL) and H₂O (0.70 mL), then the suspension was degassed with N₂ for 10 min. (dppf)PdCl₂ (26 mg, 0.0360 mmol) was added and the reaction mixture was stirred at 90° C. for 15 h. Upon cooling, CH₂Cl₂ (20 mL) was added and the mixture was dried over anhyd. MgSO₄, filtered, and concentrated. The residue was purified by silica gel chromatography (100% CH₂Cl₂ to 10% CH₂Cl₂ in MeOH) to afford the desired product as an orange solid (244 mg; 56%).

Step f: To a mixture of the product of step e (78 mg, 0.129 mmol) and (2R)-2-methylpyrrolidine (30 μL, 0.296 mmol) in DMF (2.7 mL) was added AcOH (15 μL, 0.270 mmol) and the mixture stirred at r.t. for 30 min, then NaBH(OAc)₃ (63 mg, 0.296 mmol) was added. The reaction mixture was stirred at r.t. for 14 h and carefully quenched with H₂O then sat. aq. NaHCO₃. The mixture was extracted with EtOAc (3×10 mL), then the combined organic layers were washed with brine, dried over anhyd. MgSO₄, and concentrated. Purification by silica gel chromatography (100% CH₂Cl₂ to 10% MeOH in CH₂Cl₂, 1% NH₃) to afford the desired product as an orange solid 67 mg; 37%, ˜1:1 d.r.).

Step g: To a solution of the product of step f (67 mg, 0.0994 mmol) in CH₂Cl₂ (1.4 mL) was added TFA (0.70 mL). The reaction stirred for 1 h at r.t. then concentrated. To a solution of the residue in MeOH (2.0 mL) was added DMEDA (80 μL, 0.746 mmol) and the mixture was stirred at 45° C. for 1 h. Upon cooling, the reaction was diluted with H₂O (5 mL) and CH₂Cl₂ (5 mL) and the layers were separated. The aqueous layer was extracted with 10% MeOH in CH₂Cl₂ (2×10 mL), then the combined organic layers were concentrated. Purification by reverse phase HPLC (10 to 70% ACN in H₂O, 0.1% TFA) and lyophilization provided the title compound as an off-white solid (4 mg, 6%). ¹H NMR (400 MHz, Methanol-d₄) δ 8.82 (d, J=2.1 Hz, 1H), 8.63 (d, J=2.1 Hz, 1H), 7.99-7.96 (m, 2H), 7.88 (dd, J=1.1, 0.7 Hz, 1H), 7.59 (d, J=2.0 Hz, 1H), 7.55 (dd, J=7.7, 1.9 Hz, 1H), 7.34 (d, J=7.8 Hz, 1H), 4.28-4.21 (m, 2H), 3.86-3.69 (m, 2H), 3.60 (dd, J=4.9, 3.8 Hz, 2H), 3.47-3.39 (m, 1H), 3.29-3.23 (m, 1H), 3.11-2.90 (m, 4H), 2.45-2.34 (m, 2H), 2.30 (dq, J=13.8, 7.1 Hz, 1H), 2.11-1.95 (m, 2H), 1.81-1.68 (m, 1H), 1.68-1.53 (m, 2H), 1.47 (d, J=6.6 Hz, 3H). ESI MS [M+H]⁺ for C₃₀H₃₄N₅O₃S, calcd. 544.2, found 544.2.

### Biological Example

### Measuring Intracellular Binding of Axl Inhibitors

Axl NanoBRET™ intracellular kinase assay (Promega, N2540) was performed according to manufacturer's recommendation. In brief, HEK-293 cells are transiently transfected with Axl-NanoLuc fusion vector (Promega, NV1071), utilizing Fugene HD transfection reagent (Promega, E2311) a day before experiment following manufacturer's recommendation.

The day of the assay, the cells were collected and resuspended in Opti-MEM media (ThermoFisher, 31985070) at 2e5 cells/mL concentration. Testing compounds were serial diluted and dispensed into a white 384 well polystyrene plate at 200 nL in 100% DMSO. 40 μL of resuspended cells is then added per well for a final condition of 8K cells/well with 0.5% DMSO. After one-hour compound pre-incubation at 37° C. and 5% CO₂, cells were further incubated with 0.35 μM K-5 NanoBRET tracer for two-hours at 37° C. and 5% CO₂. 20 ml of 3× substrate plus Inhibitor solution was prepared according to kits manual and added to the cells followed by a 30 second pulse centrifugation spin. The plate was then immediately read utilizing an Envision (Perkin Elmer) plate reader. The BRET signal is measured by taking the ratio of the emissions reading at 610 nm and 450 nm. Compound binding is based on the decrease BRET signal caused by the displacement of the K-5 tracer. DMSO treated activity was used as neutral control and normalized to 100% activity, and CEP-40783 control compound at 20 μM which reaches 100% inhibition was used as positive control and normalized to 0% activity. IC₅₀ value of compounds was determined by 4-parameter non-linear regression fitting of percent activity in GraphPad Prism software. Values are reported in Table 1 (Cell Binding).

### Measurement of Biochemical Compound Potency of Axl Inhibitors

Purified recombinant human AXL, TYRO3 and MER proteins were purchased from Invitrogen™. 10 nM AXL, 2 nM TYRO3 or MER were incubated with varying concentrations of compounds in 50 mM HEPES, pH 7.4, 10 mM MgCl₂, 0.01% BSA, 1 mM DTT and 2% DMSO in a total volume of 20 μl in a 384-well microplate (Corning™ #3640) at RT for 1 h. The AXL, TYRO3 and MER enzymatic reaction was initiated by transferring 10 μl of enzyme and compound mixture into 10 μl of 1.6 μM TK Substrate-biotin (HTRF® KinEASE-TK kit, Cisbio) and 1400 μM ATP pre-incubated in 50 mM HEPES, pH 7.4, 10 mM MgCl₂, 0.01% BSA, 1 mM DTT in a 384-well microplate (Corning™ #3640) at RT, giving the final reaction conditions: 5 nM AXL, 1 nM TYRO3 or MER, 800 nM TK Substrate-biotin and 700 μM ATP in 50 mM HEPES, pH 7.4, 10 mM MgCl₂, 0.01% BSA, 1 mM DTT and 1% DMSO with varying concentrations of compounds. Following 2 h incubation at RT, the AXL, TYRO3 and MER enzymatic reaction was stopped by transfer of 10 μl of reaction into 10 μl of detection mix (400 nM Streptavidin-XL665, 200-fold dilution TK Antibody-Cryptate and detection buffer, HTRF® KinEASE-TK kit, Cisbio) in a white 384-well microplate (Perkin Elmer, OptiPlate 384). After 1 h incubation at RT, the plate was put into a plate reader (Evision) to read at 665/620 nm (acceptor/donor) for HTRF. The value of the DMSO blank (MIN inhibition=100% activity) was used as a negative control. The positive control was established by adding 5 μl of enzyme and DMSO mixture into 10 μl of detection mix followed by addition of 5 μl of TK Substrate-biotin and ATP mixture (MAX inhibition=0% activity). To calculate the percent activity, Equation 1 was used. Ratio_{665/620} is the value at a given compound concentration:

$$\% Activity = \frac{{Ratio}_{665 / 620} - M A X}{MIN - M A X} \times 100 \qquad ( 1 )$$

The concentration of compound that resulted in 50% loss of the enzyme activity (IC₅₀) was calculated by GraphPad Prism using Equation 2 where N is the Hill coefficient:

$$\% Activity = Bottom + \frac{Top - B o t t o m}{1 + {( \frac{[ I ]}{{IC}_{50}} )}^{N}} \qquad ( 2 )$$

Values are reported in Table 1 (Biochemical Potency).

TABLE 1
Biochemical and cellular potency of specific examples
(IC₅₀: + means >1 μM, ++ means 100 nM to 1 μM, +++ means <100 nM)

| | |
|---|---|
| Cell Binding | Biochemical Potency |

| Ex. | AXL | AXL | MER | TYRO3 |
|---|---|---|---|---|
| 1 | +++ | +++ | +++ | +++ |
| 2 | +++ | +++ | +++ | +++ |
| 3 | ++ | +++ | +++ | +++ |
| 4 | +++ | +++ | +++ | +++ |
| 5 | +++ | +++ | +++ | +++ |
| 6 | +++ | +++ | +++ | +++ |
| 7 | +++ | +++ | +++ | +++ |
| 8 | + | ++ | | |
| 9 | +++ | +++ | +++ | +++ |
| 10 | ++ | +++ | +++ | +++ |
| 11 | ++ | +++ | +++ | +++ |
| 12 | ++ | +++ | ++ | +++ |
| 13 | ++ | +++ | ++ | +++ |
| 14 | + | + | | |
| 15 | +++ | +++ | +++ | +++ |
| 16 | ++ | +++ | ++ | +++ |
| 17 | ++ | +++ | +++ | +++ |
| 18 | + | +++ | | +++ |
| 19 | ++ | +++ | ++ | +++ |
| 20 | ++ | +++ | ++ | +++ |
| 21 | ++ | +++ | ++ | ++ |
| 22 | +++ | +++ | +++ | +++ |
| 23 | +++ | +++ | +++ | +++ |
| 24 | +++ | +++ | +++ | +++ |
| 25 | +++ | +++ | +++ | +++ |
| 26 | ++ | +++ | ++ | +++ |
| 27 | ++ | +++ | + | ++ |
| 28 | ++ | +++ | ++ | ++ |
| 29 | +++ | +++ | ++ | +++ |
| 30 | +++ | +++ | ++ | +++ |
| 31 | +++ | +++ | ++ | +++ |
| 32 | +++ | +++ | +++ | +++ |
| 33 | +++ | +++ | +++ | +++ |
| 34 | +++ | +++ | +++ | +++ |
| 35 | +++ | +++ | +++ | +++ |
| 36 | +++ | +++ | +++ | +++ |
| 37 | +++ | +++ | +++ | +++ |
| 38 | +++ | +++ | ++ | +++ |
| 39 | +++ | +++ | ++ | +++ |
| 40 | +++ | +++ | ++ | +++ |
| 41 | +++ | +++ | ++ | +++ |
| 42 | +++ | +++ | +++ | +++ |
| 43 | +++ | +++ | +++ | +++ |
| 44 | +++ | +++ | +++ | +++ |
| 45 | +++ | +++ | +++ | +++ |
| 46 | +++ | +++ | +++ | +++ |
| 47 | +++ | +++ | +++ | +++ |
| 48 | ++ | +++ | +++ | +++ |
| 49 | +++ | +++ | +++ | +++ |
| 50 | +++ | +++ | +++ | +++ |
| 51 | ++ | +++ | ++ | +++ |
| 52 | +++ | +++ | +++ | +++ |
| 53 | ++ | +++ | +++ | +++ |
| 54 | ++ | +++ | ++ | +++ |
| 55 | ++ | +++ | ++ | +++ |
| 56 | +++ | +++ | ++ | +++ |
| 57 | +++ | +++ | +++ | +++ |
| 58 | +++ | +++ | +++ | +++ |
| 59 | +++ | +++ | +++ | +++ |
| 60 | +++ | +++ | +++ | +++ |
| 61 | +++ | +++ | ++ | +++ |
| 62 | +++ | +++ | +++ | +++ |
| 63 | ++ | +++ | ++ | +++ |
| 64 | +++ | +++ | +++ | +++ |
| 65 | +++ | +++ | +++ | +++ |
| 66 | +++ | +++ | ++ | ++ |

Particular embodiments of this disclosure are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

What is claimed is:

1. A compound represented by Formula (I) 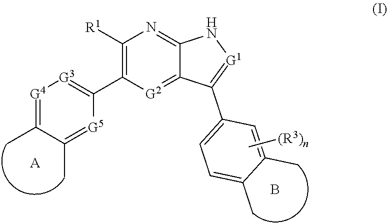
or a pharmaceutically acceptable salt thereof, wherein:
G¹ is N or CR^{G1};
G² is CR^{G2} or N;
G³ is CR^{G3} or N;
G⁴ is CR^{G4} or N;
G⁵ is CR^{G5} or N;
R^{G1} is selected from the group consisting of H, C₁₋₃ alkyl, halogen, C₁₋₃ haloalkyl and CN;
each R^{G2}, R^{G3}, R^{G4} and R^{G5} is independently selected from the group consisting of H, halo, CN, C₁₋₇ alkyl, C₃₋₇ cycloalkyl, C₁₋₃ haloalkyl, —O—C₁₋₃ alkyl, —O—C₁₋₃ haloalkyl, —NR^{a}R^{b}, and 4- to 8-membered heterocycloalkyl having 1-3 heteroatom ring vertices selected from the group consisting of O, N, and S, and wherein the cycloalkyl and heterocycloalkyl groups are substituted with 0-3 groups independently selected from halo, CN, C₁₋₄ alkyl, C₁₋₄ haloalkyl, C₁₋₄ hydroxyalkyl, —O—C₁₋₄ alkyl, and OH;
R¹ is selected from the group consisting of H, C₁₋₄ alkyl and NH₂;
A is a fused ring selected from the group consisting of azepane, piperidine, cycloheptane, cyclohexane, cyclopentane, 1,4-oxazepane, oxepane, tetrahydropyran, 1,4-diazepane, bicyclo[4.2.1]nonane, bicyclo[4.1.1]octane, spiro[4.6]undecane, 1-azaspiro[4.6]undecane and cyclooctane, each of which is unsubstituted or substituted with from 1 to 4 R², and further substituted with 0 or 1 oxo (═O) which is adjacent to a nitrogen atom;
B is a fused ring selected from the group consisting of 1,4-oxazepane, cycloheptane, tetrahydropyran, isothiazolidine 1,1-dioxide, oxepane, 1,4,5-oxathiazepane 4,4-dioxide, cyclohexane, cyclopentane, azepane, pyrrolidine, piperidine, piperazine, morpholine, diazepane, and 1,3-dioxolane, each of which is unsubstituted or substituted with from 1 to 4 R⁴; and further substituted with 0 or 1 oxo (═O) which is adjacent to a nitrogen atom;
each R² is independently selected from the group consisting of halo, OH, C₁₋₇ alkyl, C₃₋₇ alkenyl, C₃₋₇ alkynyl, C₃₋₇ cycloalkyl, —C(O)—C₁₋₇ alkyl, —C(O)—C₃₋₇ cycloalkyl, —C(O)—C₁₋₇ alkylene-OH, —Y¹—O—C₁₋₇ alkyl, —Y¹—O—C₃₋₇ cycloalkyl, —NR^{a}R^{b}, —S(O)₂—C₁₋₇ alkyl, —S(O)₂—C₃₋₇ cycloalkyl, —C(O)NR^{a}R^{b}, 4- to 8-membered heterocycloalkyl, and —NR^{a}—(4- to 8-membered heterocycloalkyl), wherein the 4- to 8-membered heterocycloalkyl has 1-3 heteroatom ring vertices selected from the group consisting of O, N, and S, and wherein the cycloalkyl and heterocycloalkyl groups are substituted with from 0-3 groups independently selected from halo, CN, C₁₋₄ alkyl, C₁₋₄ haloalkyl, C₁₋₄ hydroxyalkyl, —O—C₁₋₄ alkyl, and OH;
the subscript n is 0, 1, 2 or 3;
each R³ is independently selected from the group consisting of halogen, CN, C₁₋₇ alkyl, C₂₋₇ alkenyl, C₃₋₇ alkynyl, C₃₋₇ cycloalkyl, C₁₋₆ haloalkyl, C₁₋₆ hydroxyalkyl, C₁₋₆ halohydroxyalkyl, —O—C₁₋₇ alkyl, —O—C₃₋₇ cycloalkyl, —O—C₁₋₆ haloalkyl, —X¹—CN, —X¹—O—C₁₋₇ alkyl, —O—Y¹—O—C₁₋₇ alkyl, —NR^{a}R^{b}, —X¹— NR^{a}R, —O—Y¹—NR^{a}R^{b}, —C(O)—NR^{a}R^{b}, —S(O)₂—NR^{a}R^{b}, —S(O)(NH)—C₁₋₇ alkyl, —S(O)₂—C₁₋₇ alkyl, —S(O)₂—C₁₋₇ haloalkyl, —S(O)₂—C₃₋₇ cycloalkyl, —S(O)₂—Y¹—O—C₁₋₃ alkyl, —S(O)₂-(4- to 8-membered heterocycloalkyl), —C(O)NH-(4- to 8-membered heterocycloalkyl), 4- to 8-membered heterocycloalkyl, and —O—X¹-(4- to 8-membered heterocycloalkyl), wherein the 4- to 8-membered heterocycloalkyl has 1-2 heteroatom ring vertices selected from the group consisting of O, N, and S; and wherein the cycloalkyl and heterocycloalkyl groups are substituted with 0-3 groups independently selected from halo, CN, C₁₋₄ alkyl, C₁₋₄ haloalkyl, C₁₋₄ hydroxyalkyl, —O—C₁₋₄ alkyl, and OH;
each R⁴ is independently selected from the group consisting of H, halogen, hydroxy, CN, C₁₋₇ alkyl, C₂₋₇ alkenyl, C₃₋₇ alkynyl, C₃₋₇ cycloalkyl, C₁₋₆ haloalkyl, C₁₋₆ hydroxyalkyl, C₁₋₆ halohydroxyalkyl, —O—C₁₋₇ alkyl, —O—C₃₋₇ cycloalkyl, —O—C₁₋₆ haloalkyl, —X¹—CN, —X¹—O—C₁₋₇ alkyl, —S(O)₂—C₁₋₄ alkyl, —S(O)₂—C₃₋₇ cycloalkyl, —C(O)NR^{a}R^{b}, —NR^{a}R^{b}, —NR^{a}—C(O)—C₁₋₇ alkyl, —NR^{a}—C(O)—C₃₋₇ cycloalkyl, —NR^{a}—S(O)₂—C₁₋₇ alkyl, and —NR^{a}—S(O)₂—C₃₋₇ cycloalkyl, wherein —NR^{a}R^{b}, —NR^{a}—C(O)—C₁₋₇ alkyl, —NR^{a}—C(O)—C₃₋₇ cycloalkyl, —NR^{a}—S(O)₂—C₁₋₇ alkyl, and —NR^{a}—S(O)₂—C₃₋₇ cycloalkyl groups are not directly attached to a nitrogen ring vertex to form a N—N bond;
or two R⁴ attached to a common carbon are combined to form a C₃₋₆ spirocycloalkyl which is unsubstituted or substituted with 1-3 members independently selected from F, Cl, OH, and CH₃;
each X¹ is C₁₋₇ alkylene or C₃₋₇ cycloalkylene;
each Y¹ is C₂₋₇ alkylene or C₃₋₇ cycloalkylene, wherein two attached heteroatoms are not attached to a common carbon atom;
each R^{a} and R^{b} are independently selected from group consisting of H, C₁₋₇ alkyl, C₁₋₇ haloalkyl, C₁₋₄ alkoxyC₁₋₄alkyl, and C₃₋₇ cycloalkyl; or
R^{a} and R^{b} together with the nitrogen to which they are attached form a 4-8 membered heterocycloalkyl ring having 0-2 additional heteroatom ring vertices selected from the group consisting of O, N, and S, and substituted with 0-3 groups independently selected from halogen, CN, C₁₋₄ alkyl, C₁₋₄ haloalkyl, C₁₋₄ hydroxyalkyl, —O—C₁₋₄ alkyl, oxo and OH.

2. The compound of claim 1, or a pharmaceutically acceptable salt thereof, wherein G¹ is N.

3. The compound of claim 1, or a pharmaceutically acceptable salt thereof, wherein G¹ is CH.

4. The compound of any one of claims 1-3, or a pharmaceutically acceptable salt thereof, wherein G² is CH or CF.

5. The compound of any one of claims 1-4, or a pharmaceutically acceptable salt thereof, wherein G³ is selected from the group consisting of CH, CF, C(CH₃), and N.

6. The compound of any one of claims 1-5, or a pharmaceutically acceptable salt thereof, wherein G⁴ is CH, CCl, or N.

7. The compound of any one of claims 1-6, or a pharmaceutically acceptable salt thereof, wherein G⁵ is CH or N.

8. The compound of claim 1, or a pharmaceutically acceptable salt thereof wherein G¹ is N, and G² is CH.

9. The compound of claim 8, or a pharmaceutically acceptable salt thereof, wherein G³ is CH.

10. The compound of claim 9, or a pharmaceutically acceptable salt thereof, wherein G⁴ is CH.

11. The compound of claim 10, or a pharmaceutically acceptable salt thereof, wherein G⁵ is CH.

12. The compound of any one of claims 1-11, or a pharmaceutically acceptable salt thereof, wherein fused ring A has a formula selected from the group consisting of: 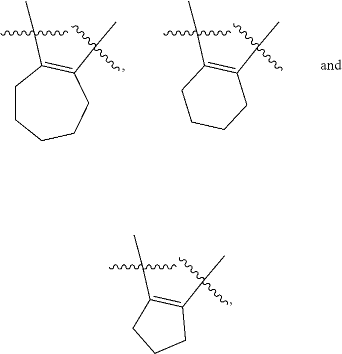 [SMILES: *C1=C(*)CCCCC1 ; *C1=C(*)CCCC1 ; *C1=C(*)CCC1]
each of which is substituted with from 1 to 4 R².

13. The compound of claim 12, or a pharmaceutically acceptable salt thereof, wherein fused ring A has the formula:

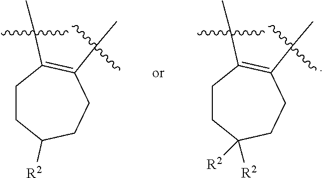

14. The compound of claim 13, or a pharmaceutically acceptable salt thereof, wherein one R² is —NR^{a}R^{b}.

15. The compound of claim 14, or a pharmaceutically acceptable salt thereof, wherein one R² is pyrrolidinyl, which is unsubstituted or substituted with from 1-3 substituents independently selected from the group consisting of halogen, CN, C₁₋₄ alkyl, C₁₋₄ haloalkyl, C₁₋₄ hydroxyalkyl, —O—C₁₋₄ alkyl, oxo and OH.

16. The compound of any one of claims 1-12, or a pharmaceutically acceptable salt thereof, wherein ring B is selected from the group consisting of 1,4-oxazepane, tetrahydropyran, isothiazolidine 1,1-dioxide, 1,4,5-oxathiazepane 4,4-dioxide, azepane, and pyrrolidine, each of which is unsubstituted or substituted with from 1 to 3 R⁴; and further substituted with 0 or 1 oxo (═O) which is adjacent to a nitrogen atom.

17. The compound of any one of claims 1-16, or a pharmaceutically acceptable salt thereof, wherein each R⁴ is independently selected from the group consisting of halogen, C₁₋₄ alkyl, C₁₋₄ haloalkyl and OH, or two R⁴ attached to a common carbon are combined to form a C₃₋₆ spirocycloalkyl which is unsubstituted or substituted with 1-3 members independently selected from F, Cl, OH, and CH₃.

18. The compound of any one of claims 1-11, or a pharmaceutically acceptable salt thereof, wherein fused ring A has a formula selected from the group consisting of: 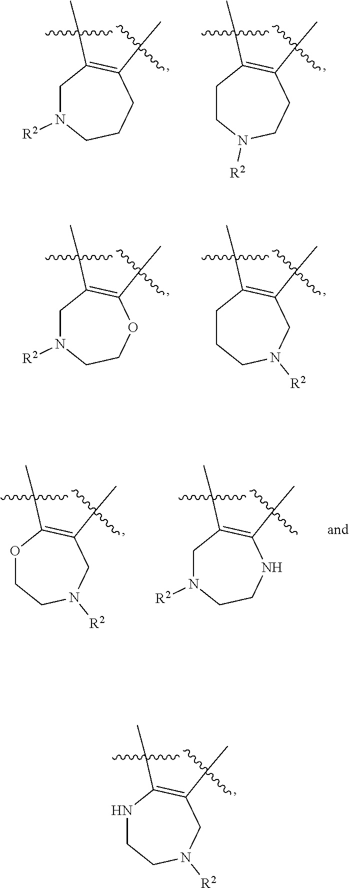
each of which is optionally substituted with an additional 1 to 2 R².

19. The compound of claim 18, or a pharmaceutically acceptable salt thereof, wherein fused ring A has the formula:

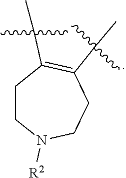

20. The compound of claim 18 or 19, or a pharmaceutically acceptable salt thereof, wherein the R², attached to nitrogen is selected from the group consisting of C₁₋₇ alkyl, C₃₋₇ cycloalkyl, —C(O)—C₁₋₇ alkyl, —C(O)—C₃₋₇ cycloalkyl, —C(O)—C₁₋₇ alkylene-OH, —Y¹—O—C₁₋₇ alkyl, —Y¹—O—C₃₋₇ cycloalkyl, —S(O)₂—C₁₋₇ alkyl, —S(O)₂—C₃₋₇ cycloalkyl, —C(O)NR^{a}R^{b}, and 4- to 8-membered heterocycloalkyl, wherein 4- to 8-membered heterocycloalkyl has 1-3 heteroatom ring vertices selected from the group consisting of O, N, and S, and wherein the cycloalkyl and heterocycloalkyl groups are substituted with from 0-3 groups independently selected from halo, CN, C₁₋₄ alkyl, C₁₋₄ haloalkyl, C₁₋₄ hydroxyalkyl, —O—C₁₋₄ alkyl, and OH.

21. The compound of any one of claims 1-11, or 18-20, or a pharmaceutically acceptable salt thereof, wherein fused ring B has a formula selected from the group consisting of: 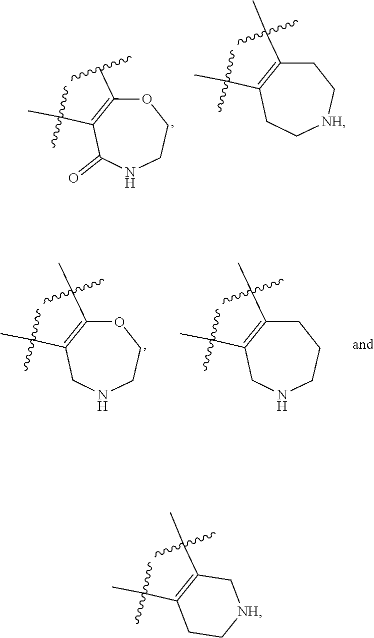 [SMILES: *C1=C(*)C(=O)NCCO1 ; *C1=C(*)CCNCC1 ; *C1=C(*)OCCNC1 ; *C1=C(*)CNCCC1 ; *C1=C(*)CNCC1]
each of which is unsubstituted or substituted with 1 to 2 R⁴.

22. The compound of claim 21, or a pharmaceutically acceptable salt thereof wherein fused ring B is unsubstituted.

23. The compound of claim 21, or a pharmaceutically acceptable salt thereof wherein fused ring B is

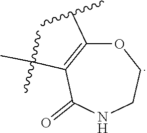 [SMILES: *C1=C(*)C(=O)NCCO1]

24. The compound of any one of claims 1-11, or 18-20, or a pharmaceutically acceptable salt thereof, wherein fused ring B has a formula selected from the group consisting of: 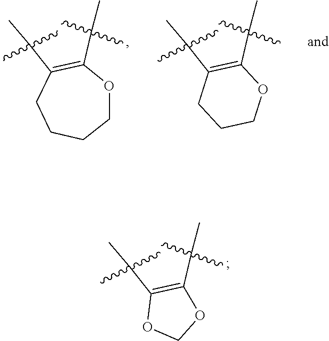 [SMILES: *C1=C(*)OCCCC1 ; *C1=C(*)OCCC1 ; *C1=C(*)OCO1]
each of which is unsubstituted or substituted with 1 to 4 R⁴.

25. The compound of claim 24, or a pharmaceutically acceptable salt thereof, wherein fused ring B is substituted with 1 to 4 R⁴, each of which is independently selected from the group consisting of halogen, C₁₋₄ alkyl, C₁₋₄ haloalkyl and OH.

26. The compound of any one of claims 1-11, or 18-20, or a pharmaceutically acceptable salt thereof, wherein fused ring B has a formula selected from the group consisting of: 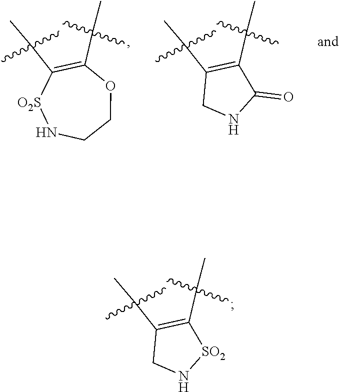 [SMILES: *C1=C(*)S(=O)(=O)NCCO1 ; *C1=C(*)C(=O)NC1 ; *C1=C(*)S(=O)(=O)NC1]
each of which is unsubstituted or substituted with 1 to 3 R⁴.

27. The compound of claim 26, or a pharmaceutically acceptable salt thereof, wherein each R⁴ is independently selected from the group consisting of C₁₋₄ alkyl and C₁₋₄ haloalkyl.

28. The compound of claim 1, or a pharmaceutically acceptable salt thereof, selected from the group consisting of:

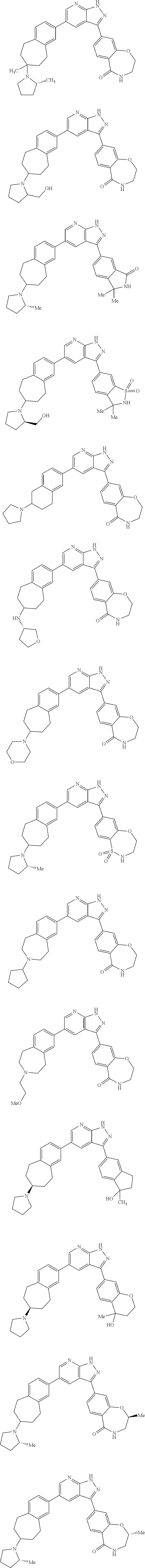

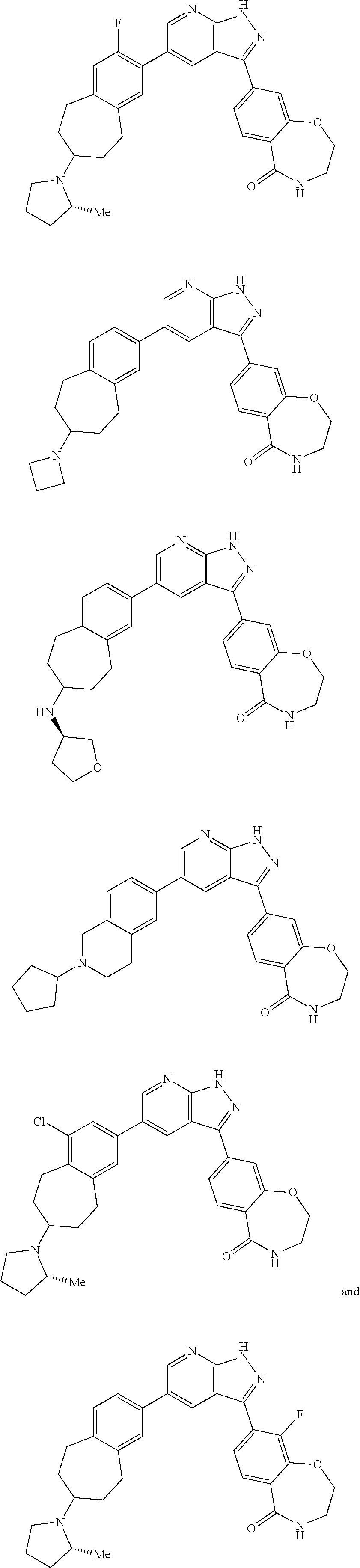

29. The compound of claim 1, or a pharmaceutically acceptable salt thereof, selected from the group consisting of

| | |
|---|---|
| 1.001 | 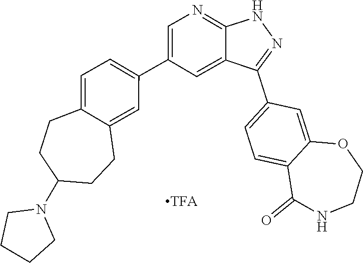 [SMILES: O=C1NCCOc2cc(-c3n[nH]c4ncc(-c5ccc6c(c5)CCC(N5CCCC5)CC6)cc34)ccc21] |
| 1.002 | 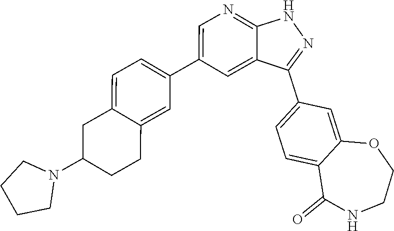 [SMILES: O=C1NCCOc2cc(-c3n[nH]c4ncc(-c5ccc6c(c5)CCC(N5CCCC5)C6)cc34)ccc21] |
| 1.003 | 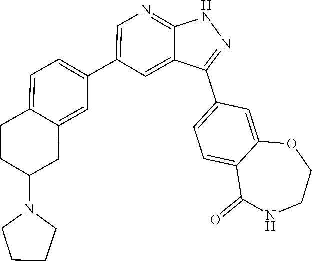 [SMILES: O=C1NCCOc2cc(-c3n[nH]c4ncc(-c5ccc6c(c5)CC(N5CCCC5)CC6)cc34)ccc21] |
| 1.004 | 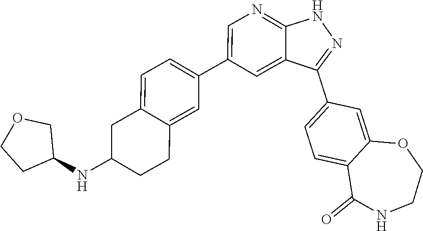 [SMILES: O=C1NCCOc2cc(-c3n[nH]c4ncc(-c5ccc6c(c5)CCC(N[C@H]5CCOC5)C6)cc34)ccc21] |
| 1.005 | 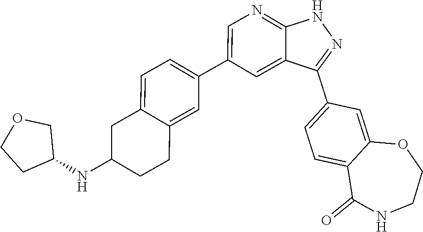 [SMILES: O=C1NCCOc2cc(-c3n[nH]c4ncc(-c5ccc6c(c5)CCC(N[C@@H]5CCOC5)C6)cc34)ccc21] |
| 1.006 | 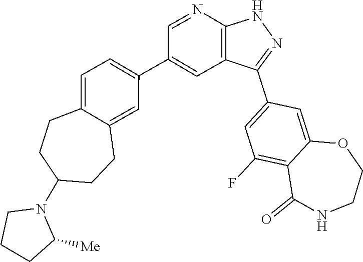 [SMILES: C[C@@H]1CCCN1C1CCc2ccc(-c3cnc4[nH]nc(-c5cc(F)c6c(c5)OCCNC6=O)c4c3)cc2CC1] |
| 1.007 | 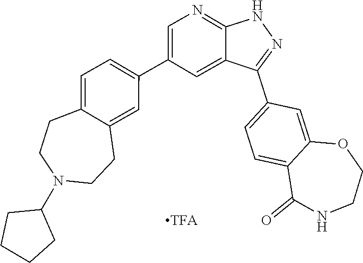 |
| 1.008 | 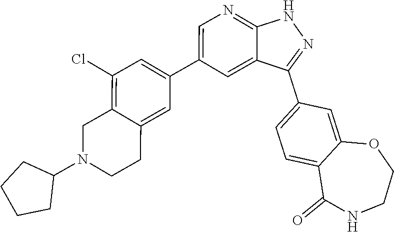 [SMILES: O=C1NCCOc2cc(-c3n[nH]c4ncc(-c5cc(Cl)c6c(c5)CCN(C5CCCC5)C6)cc34)ccc21] |
| 1.009 | 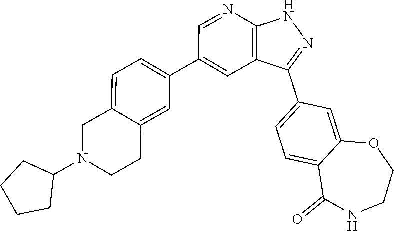 |
| 1.010 | 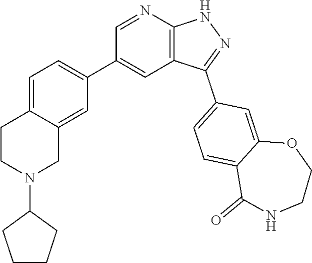 |
| 1.011 | 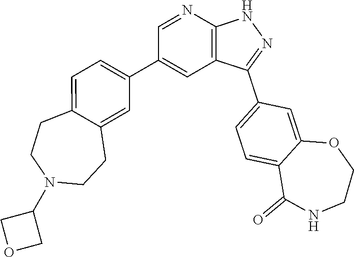 |
| 1.012 | 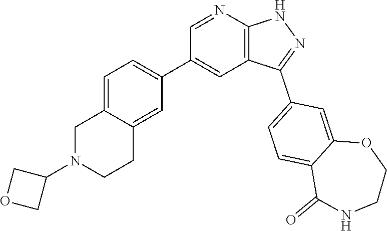 [SMILES: O=C1NCCOc2cc(-c3n[nH]c4ncc(-c5ccc6c(c5)CCN(C5COC5)C6)cc34)ccc21] |
| 1.013 | 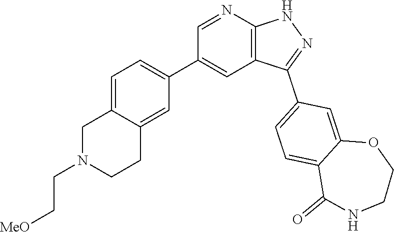 [SMILES: COCCN1CCc2cc(-c3cnc4[nH]nc(-c5ccc6c(c5)OCCNC6=O)c4c3)ccc2C1] |
| 1.014 | 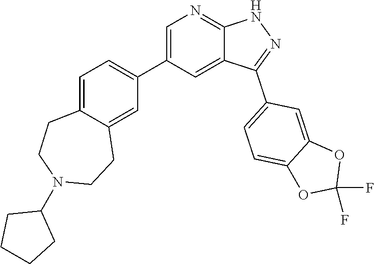 [SMILES: FC1(F)Oc2ccc(-c3n[nH]c4ncc(-c5ccc6c(c5)CCN(C5CCCC5)CC6)cc34)cc2O1] |
| 1.015 | 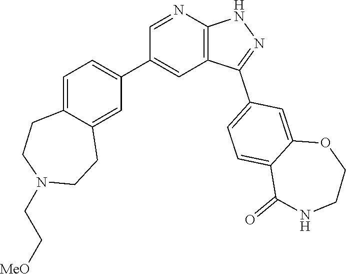 [SMILES: COCCN1CCc2ccc(-c3cnc4[nH]nc(-c5ccc6c(c5)OCCNC6=O)c4c3)cc2CC1] |
| 1.016 | 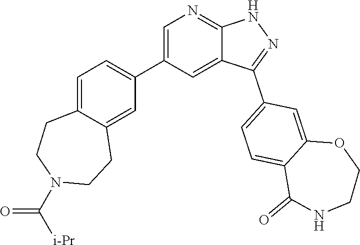 [SMILES: CC(C)C(=O)N1CCc2ccc(-c3cnc4[nH]nc(-c5ccc6c(c5)OCCNC6=O)c4c3)cc2CC1] |
| 1.017 | 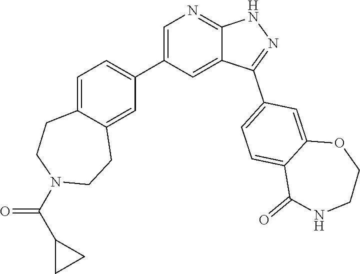 [SMILES: O=C1NCCOc2cc(-c3n[nH]c4ncc(-c5ccc6c(c5)CCN(C(=O)C5CC5)CC6)cc34)ccc21] |
| 1.018 | 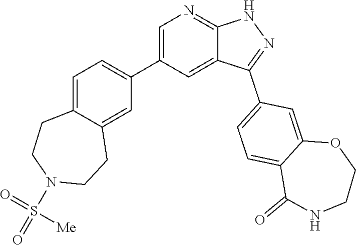 [SMILES: CS(=O)(=O)N1CCc2ccc(-c3cnc4[nH]nc(-c5ccc6c(c5)OCCNC6=O)c4c3)cc2CC1] |
| 1.019 | 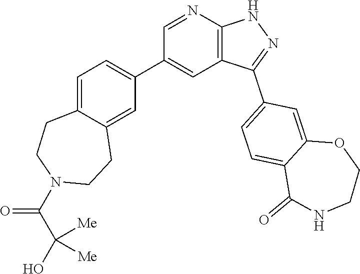 |
| 1.020 | 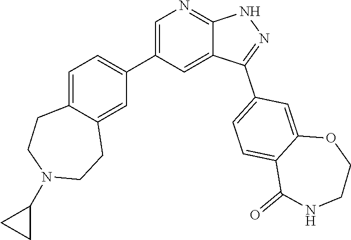 [SMILES: O=C1NCCOc2cc(-c3n[nH]c4ncc(-c5ccc6c(c5)CCN(C5CC5)CC6)cc34)ccc21] |
| 1.021 | 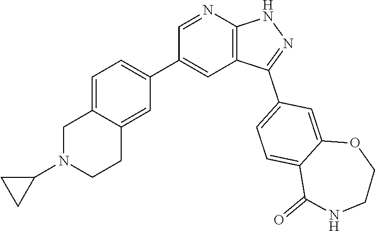 [SMILES: O=C1NCCOc2cc(-c3n[nH]c4ncc(-c5ccc6c(c5)CCN(C5CC5)C6)cc34)ccc21] |
| 1.022 | 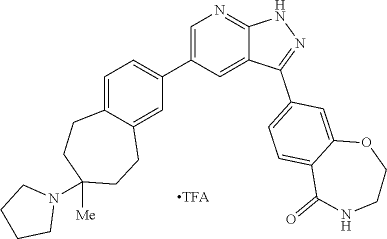 |
| 1.023 | 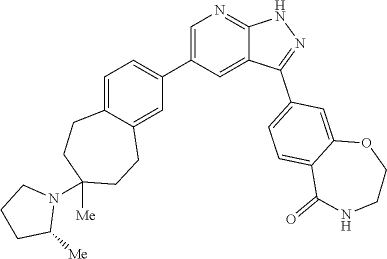 [SMILES: C[C@@H]1CCCN1C1(C)CCc2ccc(-c3cnc4[nH]nc(-c5ccc6c(c5)OCCNC6=O)c4c3)cc2CC1] |
| 1.024 | 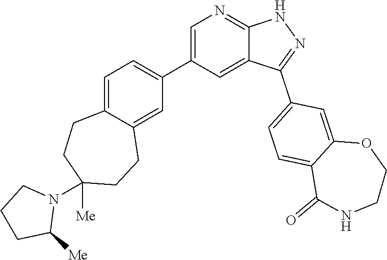 [SMILES: C[C@H]1CCCN1C1(C)CCc2ccc(-c3cnc4[nH]nc(-c5ccc6c(c5)OCCNC6=O)c4c3)cc2CC1] |
| 1.025 | 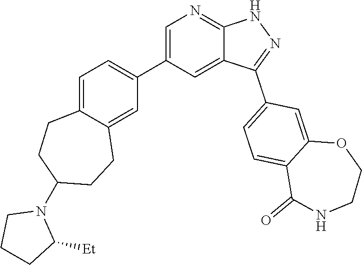 |
| 1.026 | 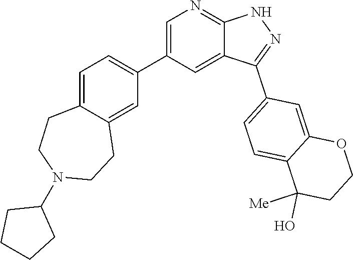 [SMILES: CC1(O)CCOc2cc(-c3n[nH]c4ncc(-c5ccc6c(c5)CCN(C5CCCC5)CC6)cc34)ccc21] |
| 1.027 | 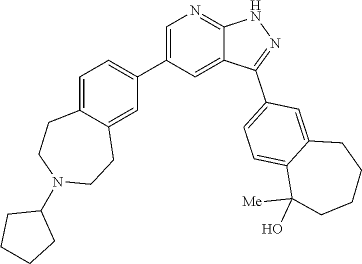 |
| 1.028 | 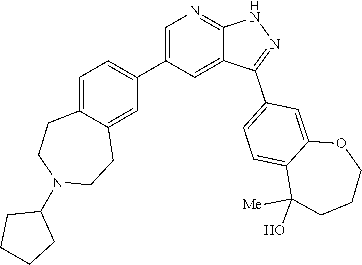 [SMILES: CC1(O)CCCOc2cc(-c3n[nH]c4ncc(-c5ccc6c(c5)CCN(C5CCCC5)CC6)cc34)ccc21] |
| 1.029 | 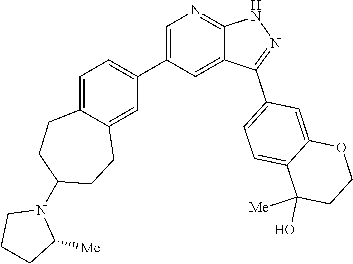 |
| 1.030 | 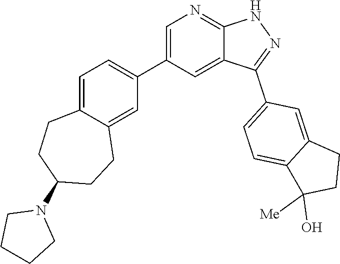 [SMILES: CC1(O)CCc2cc(-c3n[nH]c4ncc(-c5ccc6c(c5)CC[C@@H](N5CCCC5)CC6)cc34)ccc21] |
| 1.031 | 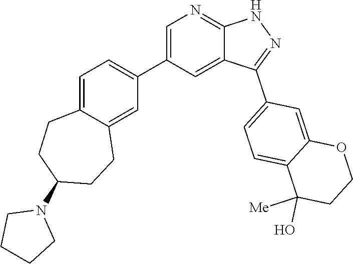 [SMILES: CC1(O)CCOc2cc(-c3n[nH]c4ncc(-c5ccc6c(c5)CC[C@@H](N5CCCC5)CC6)cc34)ccc21] |
| 1.032 | 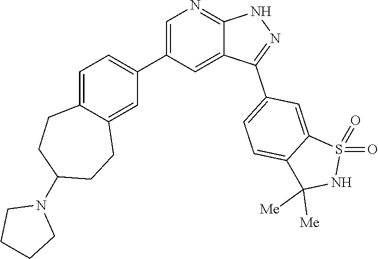 [SMILES: CC1(C)NS(=O)(=O)c2cc(-c3n[nH]c4ncc(-c5ccc6c(c5)CCC(N5CCCC5)CC6)cc34)ccc21] |
| 1.033 | 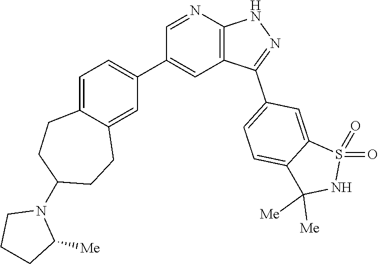 [SMILES: C[C@@H]1CCCN1C1CCc2ccc(-c3cnc4[nH]nc(-c5ccc6c(c5)S(=O)(=O)NC6(C)C)c4c3)cc2CC1] |
| 1.034 | 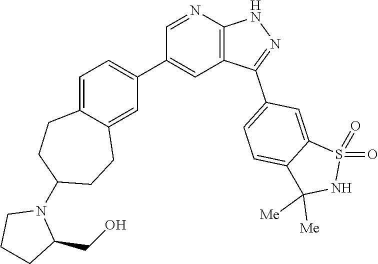 [SMILES: CC1(C)NS(=O)(=O)c2cc(-c3n[nH]c4ncc(-c5ccc6c(c5)CCC(N5CCC[C@@H]5CO)CC6)cc34)ccc21] |
| 1.035 | 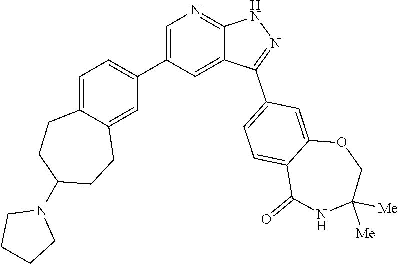 [SMILES: CC1(C)COc2cc(-c3n[nH]c4ncc(-c5ccc6c(c5)CCC(N5CCCC5)CC6)cc34)ccc2C(=O)N1] |
| 1.036 | 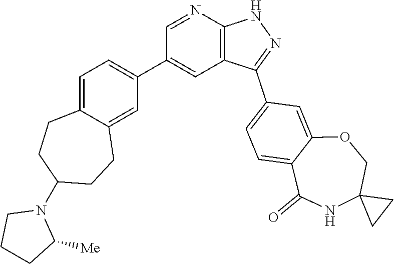 [SMILES: C[C@@H]1CCCN1C1CCc2ccc(-c3cnc4[nH]nc(-c5ccc6c(c5)OCC5(CC5)NC6=O)c4c3)cc2CC1] |
| 1.037 | 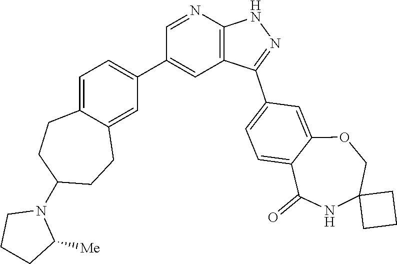 [SMILES: C[C@@H]1CCCN1C1CCc2ccc(-c3cnc4[nH]nc(-c5ccc6c(c5)OCC5(CCC5)NC6=O)c4c3)cc2CC1] |
| 1.038 | 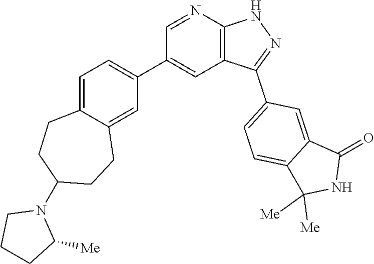 [SMILES: C[C@@H]1CCCN1C1CCc2ccc(-c3cnc4[nH]nc(-c5ccc6c(c5)C(=O)NC6(C)C)c4c3)cc2CC1] |
| 1.039 | 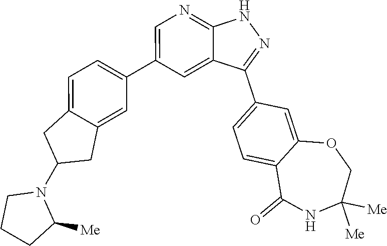 |
| 1.040 | 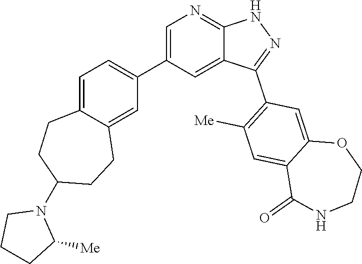 [SMILES: Cc1cc2c(cc1-c1n[nH]c3ncc(-c4ccc5c(c4)CCC(N4CCC[C@H]4C)CC5)cc13)OCCNC2=O] |
| 1.041 | 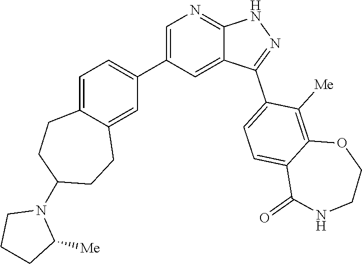 |
| 1.042 | 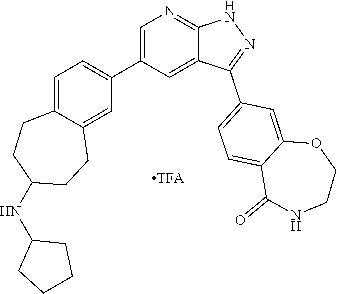 [SMILES: O=C1NCCOc2cc(-c3n[nH]c4ncc(-c5ccc6c(c5)CCC(NC5CCCC5)CC6)cc34)ccc21] |
| 1.043 | 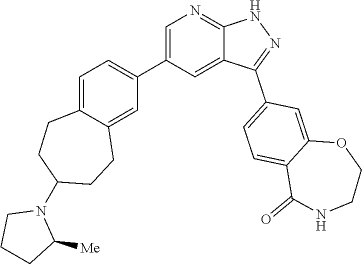 [SMILES: C[C@H]1CCCN1C1CCc2ccc(-c3cnc4[nH]nc(-c5ccc6c(c5)OCCNC6=O)c4c3)cc2CC1] |
| 1.044 | 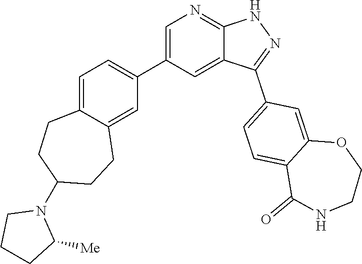 [SMILES: C[C@@H]1CCCN1C1CCc2ccc(-c3cnc4[nH]nc(-c5ccc6c(c5)OCCNC6=O)c4c3)cc2CC1] |
| 1.045 | 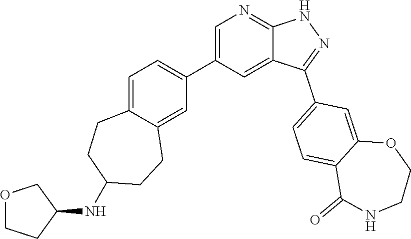 [SMILES: O=C1NCCOc2cc(-c3n[nH]c4ncc(-c5ccc6c(c5)CCC(N[C@H]5CCOC5)CC6)cc34)ccc21] |
| 1.046 | 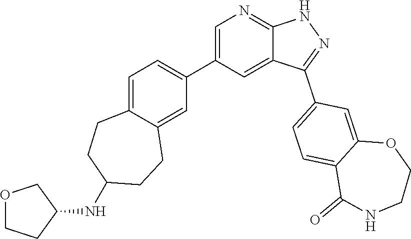 [SMILES: O=C1NCCOc2cc(-c3n[nH]c4ncc(-c5ccc6c(c5)CCC(N[C@@H]5CCOC5)CC6)cc34)ccc21] |
| 1.047 | 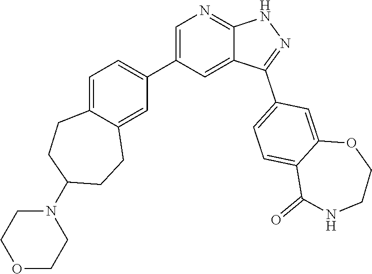 [SMILES: O=C1NCCOc2cc(-c3n[nH]c4ncc(-c5ccc6c(c5)CCC(N5CCOCC5)CC6)cc34)ccc21] |
| 1.048 | 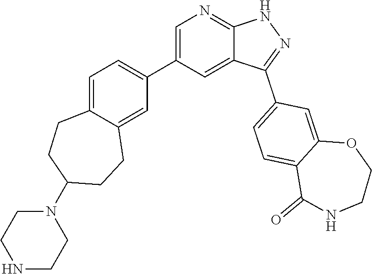 [SMILES: O=C1NCCOc2cc(-c3n[nH]c4ncc(-c5ccc6c(c5)CCC(N5CCNCC5)CC6)cc34)ccc21] |
| 1.049 | 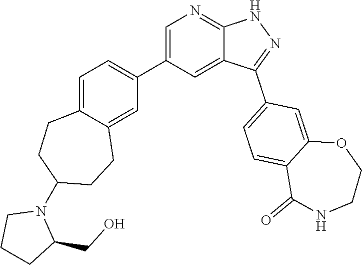 |
| 1.050 | 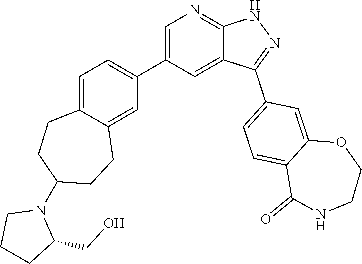 [SMILES: O=C1NCCOc2cc(-c3n[nH]c4ncc(-c5ccc6c(c5)CCC(N5CCC[C@H]5CO)CC6)cc34)ccc21] |
| 1.051 | 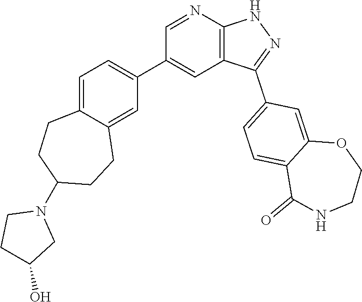 [SMILES: O=C1NCCOc2cc(-c3n[nH]c4ncc(-c5ccc6c(c5)CCC(N5CC[C@@H](O)C5)CC6)cc34)ccc21] |
| 1.052 | 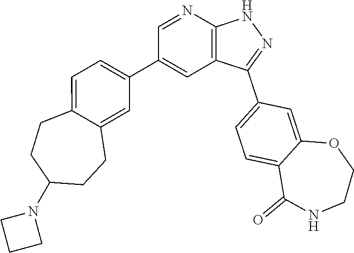 [SMILES: O=C1NCCOc2cc(-c3n[nH]c4ncc(-c5ccc6c(c5)CCC(N5CCC5)CC6)cc34)ccc21] |
| 1.053 | 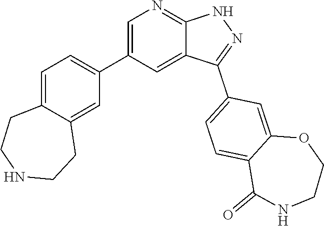 [SMILES: O=C1NCCOc2cc(-c3n[nH]c4ncc(-c5ccc6c(c5)CCNCC6)cc34)ccc21] |
| 1.054 | 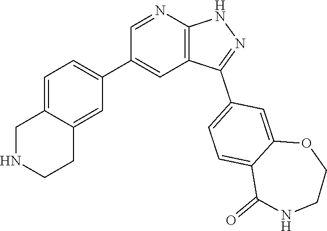 |
| 1.055 | 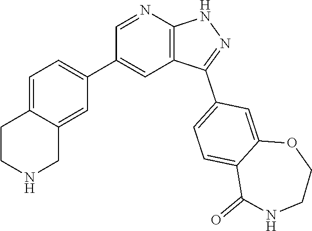 [SMILES: O=C1NCCOc2cc(-c3n[nH]c4ncc(-c5ccc6c(c5)CNCC6)cc34)ccc21] |
| 1.056 | 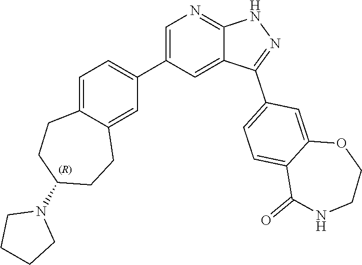 |
| 1.057 | 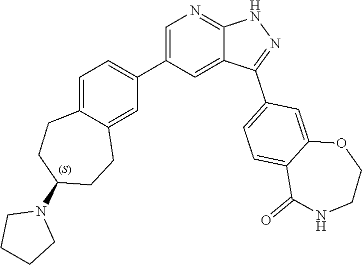 |
| 1.058 | 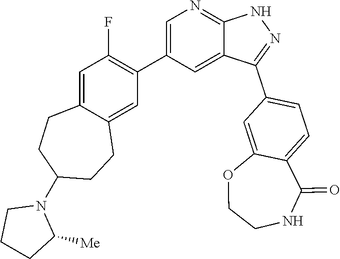 [SMILES: C[C@@H]1CCCN1C1CCc2cc(F)c(-c3cnc4[nH]nc(-c5ccc6c(c5)OCCNC6=O)c4c3)cc2CC1] |
| 1.059 | 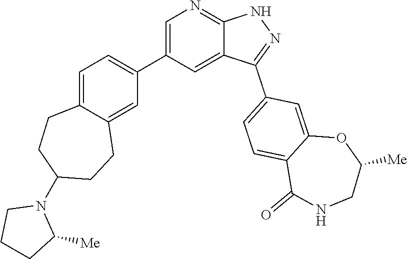 [SMILES: C[C@@H]1CNC(=O)c2ccc(-c3n[nH]c4ncc(-c5ccc6c(c5)CCC(N5CCC[C@H]5C)CC6)cc34)cc2O1] |
| 1.060 | 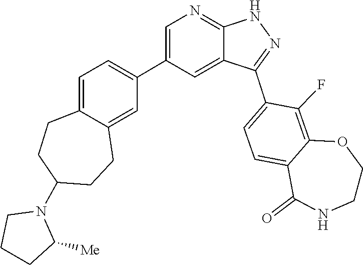 [SMILES: C[C@@H]1CCCN1C1CCc2ccc(-c3cnc4[nH]nc(-c5ccc6c(c5F)OCCNC6=O)c4c3)cc2CC1] |
| 1.061 | 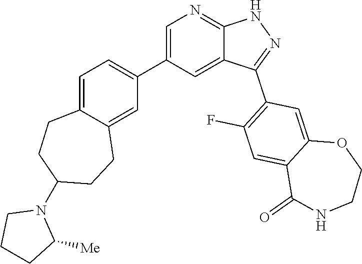 |
| 1.062 | 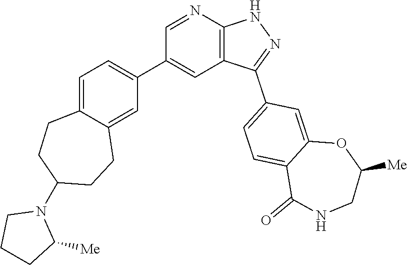 [SMILES: C[C@@H]1CCCN1C1CCc2ccc(-c3cnc4[nH]nc(-c5ccc6c(c5)O[C@@H](C)CNC6=O)c4c3)cc2CC1] |
| 1.063 | 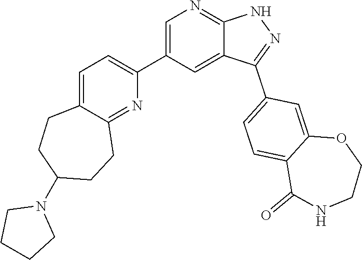 [SMILES: O=C1NCCOc2cc(-c3n[nH]c4ncc(-c5ccc6c(n5)CCC(N5CCCC5)CC6)cc34)ccc21] |
| 1.064 | 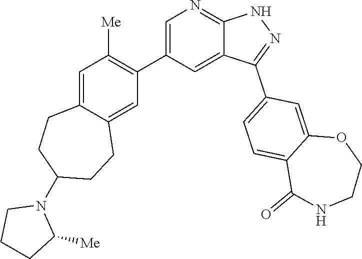 [SMILES: Cc1cc2c(cc1-c1cnc3[nH]nc(-c4ccc5c(c4)OCCNC5=O)c3c1)CCC(N1CCC[C@H]1C)CC2] |
| 1.065 | 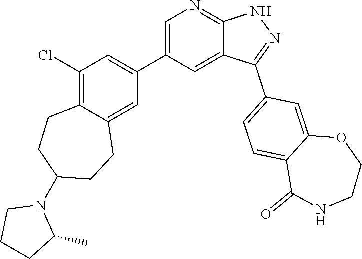 [SMILES: C[C@@H]1CCCN1C1CCc2cc(-c3cnc4[nH]nc(-c5ccc6c(c5)OCCNC6=O)c4c3)cc(Cl)c2CC1] |
| 1.066 | 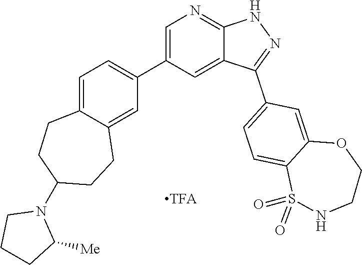 |

30. A pharmaceutical composition comprising a compound of any one of claims 1-29, or a pharmaceutically acceptable salt thereof, and a pharmaceutically acceptable excipient.

31. A method of treating a disease, disorder, or condition, mediated at least in part by AXL, said method comprising administering an effective amount of a compound of any one of claims 1-29, or a pharmaceutically acceptable salt thereof, or the pharmaceutical composition of claim 30, to a subject in need thereof.

32. The method of claim 31, wherein said compound is administered in an amount effective to reverse, slow or stop the progression of AXL-mediated dysregulation.

33. The method of any one of claims 31-32, wherein said disease, disorder, or condition is cancer.

34. The method of claim 33, wherein said cancer is a cancer of the prostate, colon, rectum, pancreas, cervix, stomach, endometrium, uterus, brain, liver, bladder, ovary, testis, head, neck, skin (including melanoma and basal carcinoma), mesothelial lining, white blood cell (including lymphoma and leukemia), esophagus, breast, muscle, connective tissue, intestine, lung (including small-cell lung carcinoma and non-small-cell lung carcinoma), adrenal gland, thyroid, kidney, or bone; or is glioblastoma, mesothelioma, renal cell carcinoma, gastric carcinoma, sarcoma (including Kaposi's sarcoma), choriocarcinoma, cutaneous basocellular carcinoma, or testicular seminoma.

35. The method of claim 33, wherein said cancer is selected from the group consisting of melanoma, colorectal cancer, pancreatic cancer, breast cancer, prostate cancer, lung cancer, leukemia, a brain tumor, lymphoma, ovarian cancer, Kaposi's sarcoma, renal cell carcinoma, head and neck cancer, esophageal cancer and urothelieal carcinoma.

36. The method of claim 31 or 32, wherein said disease, disorder, or condition is an immune-related disease, disorder or condition.

37. The method of claim 36, wherein said immune-related disease, disorder, or condition is selected from the group consisting of rheumatoid arthritis, kidney failure, lupus, asthma, psoriasis, colitis, pancreatitis, allergies, fibrosis, anemia fibromyalgia, Alzheimer's disease, congestive heart failure, stroke, aortic valve stenosis, arteriosclerosis, osteoporosis, Parkinson's disease, infections, Crohn's disease, ulcerative colitis, allergic contact dermatitis and other eczemas, systemic sclerosis and multiple sclerosis.

38. The method of any one of claims 31 to 35, further comprising at least one additional therapeutic agent.

39. The method of claim 38, wherein said at least one additional therapeutic agent comprises one or more agents independently selected from the groups consisting of inhibitors of the CD47-SIRPα pathway (e.g., anti-CD47 antibodies), inhibitors of HIF (e.g., a HIF-2α inhibitor), immune checkpoint inhibitors, agents that target the extracellular production of adenosine, radiation therapy, and chemotherapeutic agents.

40. The method of claim 38, wherein said at least one additional therapeutic agent comprises an of the CD47-SIRPα pathway.

41. The method of claim 38 or claim 40, wherein said at least one additional therapeutic agent comprises one or more immune checkpoint inhibitors that block the activity of at least one of PD-1, PD-L1, BTLA, LAG-3, a B7 family member, TIM-3, TIGIT or CTLA-4.

42. The method of claim 41, wherein said one or more immune checkpoint inhibitors comprise an immune checkpoint inhibitor that blocks the activity of PD-1 or PD-L1.

43. The method of claim 42, wherein said immune checkpoint inhibitor that blocks the activity of PD-1 or PD-L1 is selected from the group consisting of avelumab, atezolizumab, balstilimab, budigalimab, camrelizumab, cosibelimab, dostarlimab, durvalumab, emiplimab, envafolimab, ezabenlimab, nivolumab, pembrolizumab, pidilizumab, pimivalimab, retifanlimab, sasanlimab, spartalizumab, sintilmab, tislelizumab, toripalimab, and zimberelimab.

44. The method of claim 42, wherein said immune checkpoint inhibitor that blocks the activity of PD-1 or PD-L1 is is zimberelimab.

45. The method of claim 41, wherein said one or more immune checkpoint inhibitors comprise an immune checkpoint inhibitor that blocks the activity of TIGIT.

46. The method of claim 45, wherein said immune checkpoint inhibitor that blocks the activity of TIGIT is selected from AB308, domvanalimab, etigilimab, ociperlimab, tiragolumab, or vibostolimab.

47. The method of claim 45, wherein said immune checkpoint inhibitor that blocks the activity of TIGIT is AB308 or domvanalimab.

48. The method of any one of claims 38-47, wherein said at least one additional therapeutic agent comprises one or more agents that target the extracellular production of adenosine selected from the group consisting of an A₂ₐR/A_{2b}R antagonist, a CD73 inhibitor, and CD39 inhibitor.

49. The method of claim 48, wherein the one or more agents that target the extracellular production of adenosine are selected from the group consisting of etrumadenant, inupadenant, taminadenant, caffeine citrate, imaradenant, ciforadenant, and quemliclustat.

50. The method of claim 48, wherein the one or more agents that target the extracellular production of adenosine are etrumadenant and/or quemliclustat.

51. The method of any one of claims 38-50, wherein said at least one additional therapeutic agent comprises an inhibitor of HIF-2α selected from the group consisting of belzutifan, ARO-HIF2, PT-2385, and AB521.

52. The method of claim 51, wherein said inhibitor of HIF-2α is AB521.

53. The method of any one of claims 38-52, wherein said at least one additional therapeutic agent comprises a chemotherapeutic agent.

54. The method of any one of claims 38-53, wherein said at least one additional therapeutic agent comprises radiation.

55. The method of any one of claims 38-54, wherein said compound and said at least one additional therapeutic agent are administered in combination.

56. The method of any one of claims 38-54, wherein said compound and said at least one additional therapeutic agent are administered sequentially.

57. The method of any one of claims 38-54, wherein the treatment periods for the administration of the compound and the at least one additional therapeutic agent overlap.

58. A combination comprising a compound of any one of claims 1-29, or a pharmaceutically acceptable salt thereof, and at least one additional therapeutic agent.

59. The combination of claim 58, wherein the at least one additional therapeutic agent comprises one or more agents independently selected from the groups consisting of inhibitors of the CD47-SIRPα pathway (e.g., anti-CD47 antibodies), inhibitors of HIF (e.g., a HIF-2α inhibitor), immune checkpoint inhibitors, agents that target the extracellular production of adenosine, radiation therapy, and chemotherapeutic agents.

60. The combination of claim 59, wherein said at least one additional therapeutic agent comprises an inhibitor of the CD47-SIRPα pathway.

61. The combination of claim 59 or 60, wherein said at least one additional therapeutic agent comprises one or more immune checkpoint inhibitors that block the activity of at least one of PD-1, PD-L1, BTLA, LAG-3, a B7 family member, TIM-3, TIGIT or CTLA-4.

62. The combination of claim 61, wherein said one or more immune checkpoint inhibitors comprise an immune checkpoint inhibitor that blocks the activity of PD-1 or PD-L1.

63. The combination of claim 61, wherein said one or more immune checkpoint inhibitors comprise an immune checkpoint inhibitor that blocks the activity of TIGIT.

64. The combination of any one of claims 59-63, wherein the at least one additional therapeutic agent comprises a comprises a platinum-based, anthracycline-based, or taxoid based chemotherapeutic agent.

65. The combination of claim 64, wherein the chemotherapeutic agent is selected from the group consisting of cisplatin, carboplatin, oxaliplatin, doxorubicin, docetaxel, and paclitaxel.

66. A method of inhibiting the activity of AXL in a subject, comprising administering a compound according to any one of claims 1 to 29, or a pharmaceutically acceptable salt thereof, or the pharmaceutical composition of claim 30, to the subject.
